SEQUENCE LISTING (4 sequences)

SEQ ID NO: 1 - DNA, 22 bp, Artificial Sequence
  Description of Artificial Sequence Synthetic oligonucleotide
tagtcggtacgcgactaagccg

SEQ ID NO: 2 - DNA, 22 bp, Artificial Sequence
  Description of Artificial Sequence Synthetic oligonucleotide
tagtcgtcgtaacgtacgtcgg

SEQ ID NO: 3 - DNA, 21 bp, Artificial Sequence
  Description of Artificial Sequence Synthetic oligonucleotide
cggctataacgcgtcgcgtag

SEQ ID NO: 4 - DNA, 22 bp, Artificial Sequence
  Description of Artificial Sequence Synthetic oligonucleotide
tagagcgtacgcgactaacgac

(19) United States
(12) Patent Application Publication
(10) Pub. No.: US 2022/0017864 A1
(43) Pub. Date: Jan. 20, 2022

(54) METHODS OF IDENTIFYING IMMUNOMODULATORY GENES

(71) Applicant: Intima Biosciences, Inc., New York, NY (US)

(72) Inventors: Thomas HENLEY, New York, NY (US); Modassir CHOUDHRY, New York, NY (US); Lydia VINEY, New York, NY (US)

(21) Appl. No.: 17/333,903

(22) Filed: May 28, 2021

Related U.S. Application Data
(63) Continuation of application No. PCT/US2019/063383, filed on Nov. 26, 2019.
(60) Provisional application No. 62/904,283, filed on Sep. 23, 2019.
(60) Provisional application No. 62/773,767, filed on Nov. 30, 2018.

(51) Int. Cl. C12N 5/0783; C07K 14/725; C12N 15/86; C12N 15/11; C12N 9/22

(52) U.S. Cl. CPC C12N 5/0636; C07K 14/7051; C12N 15/86; C12N 2502/1157; C12N 9/22; C12N 2501/515; C12N 2501/70; C12N 15/111

(57) ABSTRACT

Disclosed herein are methods for identifying immunomodulatory genes. In some embodiments, the method comprises of screening a candidate gene comprising: a) expressing an exogenous cellular receptor, or a functional portion thereof, in a plurality of immune cells; b) introducing into said plurality of immune cells: i. a guiding polynucleic acid, or a nucleic acid encoding said guiding polynucleic acid, wherein said guiding polynucleic acid targets said candidate gene; and ii. an exogenous nuclease, or a nucleic acid encoding said exogenous nuclease; thereby generating a plurality of engineered immune cells comprising a genomic disruption in said candidate gene; c) contacting said plurality of engineered immune cells with a plurality of cells expressing a cognate antigen of said exogenous cellular receptor or a functional portion thereof, thereby performing an in vitro assay; and d) determining a readout of said in vitro assay.

### CROSS REFERENCE TO RELATED APPLICATIONS

This application is a continuation of, and claims priority to and the benefit of, International Application No. PCT/US2019/063383, filed Nov. 26, 2019, which claims priority to and the benefit of U.S. Provisional Patent Application No. 62/773,767 filed on Nov. 30, 2018, and U.S. Provisional Patent Application No. 62/904,283 filed on Sep. 23, 2019, the disclosures of each of which are hereby incorporated by reference in their entirety.

### SEQUENCE LISTING

The instant application contains a Sequence Listing which has been submitted electronically in ASCII format and is hereby incorporated by reference in its entirety. Said ASCII copy, created on May 25, 2021, is named 199827-743301_SL.txt and is 1,234 bytes in size.

### BACKGROUND

Immune responses directed against cancer cells can be important in limiting the growth or spread of cancer. Some cancerous cells, however, can negatively regulate immune responses, which can contribute to cancer cell survival and spread. An immune response can be down-regulated through mechanisms involving immunomodulatory genes. The identification of immunomodulatory genes can lead to the development of new treatments for cancer, autoimmune diseases, etc. However, new high throughput methods of identifying immunomodulatory genes are needed.

### SUMMARY

In one aspect, described herein are methods of screening a plurality of single candidate genes, said method comprising: a) expressing an exogenous cellular receptor, or a functional fragment thereof, in a plurality of separate populations of immune cells, wherein each population comprises a plurality of immune cells; b) introducing into each of said separate populations of immune cells a CRISPR system that comprises: i) a guide nucleic acid that binds a portion of a single candidate gene, wherein said single candidate gene is different for each of said separate populations of immune cells; and ii) an exogenous nuclease, or a nucleic acid encoding said exogenous nuclease; thereby generating a plurality of separate populations of engineered immune cells that comprise a genomic disruption in said single candidate gene, wherein said genomic disruption that suppresses expression of said single candidate gene; c) performing an in vitro assay that comprises contacting said plurality of engineered immune cells with a plurality of cells expressing a cognate antigen of said exogenous cellular receptor or said functional fragment thereof in vitro; and d) obtaining a readout from said in vitro assay, to thereby determine an effect of said genomic disruption that suppresses expression of said single candidate gene on said plurality of separate populations of engineered immune cells.

In some embodiments, said readout comprises determining a level of cytolytic activity of each of said plurality of separate populations of engineered immune cells. In some embodiments, said level of cytolytic activity is determined by a chromium release assay, an electrical impedance assay, time-lapse microscopy, or a co-culture assay.

In some embodiments, said readout comprises determining a level of proliferation of each of said plurality of separate populations of engineered immune cells. In some embodiments, said level of proliferation is determined by a Carboxyfluorescein Succinimidyl Ester (CFSE) assay, microscopy, an electrical impedance assay, or flow cytometry.

In some embodiments, said readout comprises determining a level of a factor expressed by each of said plurality of separate populations of engineered immune cells. In some embodiments, said factor is a protein. In some embodiments, said protein is secreted from said population of engineered immune cells. In some embodiments, said protein is a cytokine or chemokine. In some embodiments, said protein is a cell surface protein. In some embodiments, said expression is determined by flow cytometry, western blot, or ELISA.

In some embodiments, at least 60%, 65%, 70%, 75%, 80%, 85%, 90%, 95%, or 99% of immune cells of each of said separate populations of immune cells comprise said genomic disruption, in the absence of a selection step. In some embodiments, at least 80% of immune cells of each of said separate populations of immune cells comprise said genomic disruption, in the absence of a selection step. In some embodiments, at least 90% of immune cells of each of said separate populations of immune cells comprise said genomic disruption, in the absence of a selection step. In some embodiments, said percentage of immune cells of each of said separate populations of immune cells is determined by Tracking of Indels by Decomposition (TIDE) analysis.

In some embodiments, said exogenous cellular receptor is integrated into the genome of said plurality of separate populations of immune cells. In some embodiments, said exogenous cellular receptor is integrated into an endogenous gene sequence that encodes an endogenous cellular receptor. In some embodiments, said exogenous cellular receptor is integrated into a safe harbor site. In some embodiments, said safe harbor site is an AAVS site (e.g., AAVS1, AAVS2), CCR5, or hROSA26. In some embodiments, said exogenous cellular receptor is integrated into a portion of a gene that encodes a protein that functions as a negative regulator of an immune response of said plurality of immune cells.

In some embodiments, at least 50%, 60%, 65%, 70%, 75%, 80%, 85%, 90%, 95%, or 99% of immune cells of each of said separate populations of immune cells express said exogenous cellular receptor, in the absence of a selection step. In some embodiments, at least 70% of immune cells of each of said separate populations of immune cells express said exogenous cellular receptor, in the absence of a selection step. In some embodiments, at least 80% of immune cells of each of said separate populations of immune cells express said exogenous cellular receptor, in the absence of a selection step. In some embodiments, at least 90% of immune cells of each of said separate populations of immune cells express said exogenous cellular receptor, in the absence of a selection step. In some embodiments, said percentage of immune cells of each of said separate populations of immune cells is determined by flow cytometry or sequencing.

In some embodiments, said genomic disruption is a double strand break. In some embodiments, said nuclease is introduced using electroporation. The method of any preceding claim, wherein said nuclease is an endonuclease. In some embodiments, said endonuclease is selected from the group consisting of Cas1, Cas1B, Cas2, Cas3, Cas4, Cas5, Cas6, Cas7, Cas8, Cas9, Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx1S, Csf1, Csf2, CsO, Csf4, Cpf1, c2c1, c2c3, and Cas9HiFi. In some embodiments, said endonuclease is Cas9. In some embodiments, said guide nucleic acid is a guide ribonucleic acid (gRNA). In some embodiments, said guide nucleic acid comprises a phosphorothioate (PS) linkage, a 2′-fluoro (2′-F) modification, a 2′-O-methyl (2′-O-Me) linkage, a 2-O-Methyl 3phosphorothioate linkage, a S-constrained ethyl (cEt) modification, or any combination thereof. In some embodiments, said guide nucleic acid is introduced using electroporation.

In some embodiments, said exogenous cellular receptor is introduced using electroporation. In some embodiments, said exogenous cellular receptor is introduced using a viral vector. In some embodiments, said viral vector is an adeno-associated virus (AAV) vector. In some embodiments, said AAV vector is selected from the group consisting of a recombinant AAV (rAAV) vector, a hybrid AAV vector, a chimeric AAV vector, a self-complementary AAV (scAAV) vector, a modified AAV vector, and any combination thereof. In some embodiments, said AAV vector is a chimeric AAV vector. In some embodiments, said chimeric AAV vector comprises a modification in at least one AAV capsid gene sequence.

In some embodiments, said exogenous cellular receptor is a T-cell receptor (TCR), B cell receptor (BCR), NK cell receptor, dendritic cell receptor, monocyte receptor, macrophage receptor, neutrophil receptor, eosinophil receptor, or a chimeric antigen receptor (CAR). In some embodiments, said exogenous cellular receptor is a T-cell receptor (TCR).

In some embodiments, said single gene is an immunomodulatory gene. In some embodiments, said single gene is a candidate immune checkpoint gene.

In some embodiments, said method further comprises cryopreserving said separate populations of engineered immune cells. In some embodiments, said method further comprises processing said readout to identify a candidate immunomodulatory gene.

In some embodiments, said processing comprises determining a criterion from at least one of: cytolytic activity, gene expression of said candidate immunomodulatory gene, intracellular location of a protein encoded by said candidate immunomodulatory gene, loss-of-function association with a human disease of said candidate immunomodulatory gene, a guide nucleic acid score of a guide nucleic acid that binds to a portion of said candidate immunomodulatory gene, existing drugs in development that target said candidate immunomodulatory gene, existing drugs that target said candidate immunomodulatory gene, or loss-of-function phenotype of said candidate immunomodulatory gene, or any combination thereof.

In some embodiments, said processing comprises determining a criterion from at least two, three, four, five, six, seven, or eight of: cytolytic activity, gene expression of said candidate immunomodulatory gene, intracellular location of a protein encoded by said candidate immunomodulatory gene, loss-of-function association with a human disease of said candidate immunomodulatory gene, a guide nucleic acid score of a guide nucleic acid that binds to a portion of said candidate immunomodulatory gene, existing drugs in development that target said candidate immunomodulatory gene, existing drugs that target said candidate immunomodulatory gene, or loss-of-function phenotype of said candidate immunomodulatory gene, or any combination thereof.

In some embodiments, said processing comprises ranking at least two candidate immunomodulatory genes according to said at least one criterion to produce ranked candidate immunomodulatory genes. In some embodiments, said processing comprises ranking at least 10, 100, 200, 300, 400, 500, 600, 700, 800, 900, 1000, 5000, 10000, 50000, or 100000 candidate immunomodulatory genes according to said at least two, three, four, five, six, seven, or eight criterion.

In some embodiments, said processing comprises ranking at least two candidate immunomodulatory genes according to said at least two, three, four, five, six, seven, or eight criterion to produce ranked candidate immunomodulatory genes. In some embodiments, said processing comprises ranking at least 10, 100, 200, 300, 400, 500, 600, 700, 800, 900, 1000, 5000, 10000, 50000, or 100000 candidate immunomodulatory genes according to said at least one criterion.

In some embodiments, said processing comprises ranking at least 10, 100, 200, 300, 400, 500, 600, 700, 800, 900, 1000, 5000, 10000, 50000, or 100000 candidate immunomodulatory genes according to said at least one criterion.

In some embodiments, said method further comprises selecting a top 10, 20, 30, 40, or 50 of said ranked candidate immunomodulatory genes to thereby generate a ranked output.

In some embodiments, said method further comprises identifying at least one of a gene family, a gene function, or an intracellular signaling pathway from said ranked output, to thereby generate an analyzed ranked output.

In some embodiments, said method further comprises correlating cytolytic activity of said analyzed ranked output, to thereby generate a cytolytic-correlated ranked output.

In some embodiments, said method further comprises ranking said candidate immunomodulatory genes from said cytolytic-correlated ranked output according to said intracellular location of a protein encoded by said candidate immunomodulatory gene.

In some embodiments, said method further comprises ranking said candidate immunomodulatory genes from said cytolytic-correlated ranked output according to said existing drug in development that targets said candidate immunomodulatory gene and said existing drug against said candidate immunomodulatory gene.

In some embodiments, each of said populations of engineered immune cells comprises a plurality of T cells, tumor infiltrating lymphocytes (TILs), NK cells, B cell, dendritic cells, monocytes, macrophages, neutrophils, or eosinophils.

In some embodiments, each of said populations of engineered immune cells comprises a plurality of T cells. In some embodiments, said plurality of T cells comprises a plurality of CD8+ T cells. In some embodiments, said plurality of T cells comprises a plurality of CD4+ T cells. In some embodiments, said plurality of T cells comprises a plurality of CD4+ T cells and CD8+ T cells.

In some embodiments, each of said populations of engineered immune cells comprises a plurality of human cells. In some embodiments, each of said populations of engineered immune cells comprises a plurality of primary cells. In some embodiments, each of said populations of engineered immune cells comprises a plurality of ex vivo cells.

The method of any preceding claim, wherein said plurality of separate populations of immune cells comprises at least 10, 20, 30, 40, 50, 60, 70, 80, 90, 100, 200, 300, 400, 500, 600, 700, 800, 900, 1000, 5000, 10000, 50000, or 100000 separate populations of immune cells.

In some embodiments, said each of said populations of engineered immune cells comprises a transgene that encodes for a protein that improves immunomodulatory function of said engineered immune cells. In some embodiments, said transgene is integrated in the genome of said engineered immune cells.

In some embodiments, said transgene is integrated into a safe harbor site.

In some embodiments, said safe harbor site is site is an AAVS site (e.g., AAVS1, AAVS2), CCR5, or hROSA26. In some embodiments, said transgene is integrated into a portion of a gene that encodes a protein that functions as a negative regulator of an immune response of said plurality of immune cells. In some embodiments, said each of said populations of engineered immune cells comprises a genetic modification that enhances expression of a gene that encodes for a protein that improves immunomodulatory function of said engineered immune cells

In some embodiments, said plurality of cells that express said cognate antigen are cancer cells. In some embodiments, said cancer cells are primary cancer cells or from a cancer cell line. In some embodiments, said cancer cells comprise a genomic disruption in at least one gene. In some embodiments, said genomic disruption is mediated by a CRISPR system that comprises a gRNA that binds to a portion of said gene and a nuclease that mediates cleavage of genomic DNA.

In some embodiments, said genomic disruption is a double strand break.

In some embodiments, said at least one gene encodes a protein that that a negative regulator of an immune response. In some embodiments, said protein is a ligand of a checkpoint inhibitor. In some embodiments, said protein is a ligand of a checkpoint inhibitor selected from the group consisting of programmed cell death 1 (PD-1), cytotoxic T-lymphocyte-associated protein 4 (CTLA4), interleukin 10 receptor subunit alpha (IL10RA), interleukin 10 receptor subunit beta (IL10RB), adenosine A2a receptor (ADORA), CD276, V-set domain containing T cell activation inhibitor 1 (VTCN1), B and T lymphocyte associated (BTLA), indoleamine 2,3-dioxygenase 1 (IDO1), killer cell immunoglobulin-like receptor, three domains, long cytoplasmic tail, 1 (KIR3DL1), lymphocyte-activation gene 3 (LAG3), hepatitis A virus cellular receptor 2 (HAVCR2), V-domain immunoglobulin suppressor of T-cell activation (VISTA), natural killer cell receptor 2B4 (CD244), hypoxanthine phosphoribosyltransferase 1 (HPRT), adeno-associated virus integration site 1 (AAVS1), or chemokine (C—C motif) receptor 5 (gene/pseudogene) (CCR5), CD160 molecule (CD160), T-cell immunoreceptor with Ig and ITIM domains (TIGIT), CD96 molecule (CD96), cytotoxic and regulatory T-cell molecule (CRTAM), leukocyte associated immunoglobulin like receptor 1 (LAIR1), sialic acid binding Ig like lectin 7 (SIGLEC7), sialic acid binding Ig like lectin 9 (SIGLEC9), tumor necrosis factor receptor superfamily member 10b (TNFRSF10B), tumor necrosis factor receptor superfamily member 10a (TNFRSF10A), caspase 8 (CASP8), caspase 10 (CASP10), caspase 3 (CASP3), caspase 6 (CASP6), caspase 7 (CASP7), Fas associated via death domain (FADD), Fas cell surface death receptor (FAS), transforming growth factor beta receptor II (TGFBRII), transforming growth factor beta receptor I (TGFBR1), SMAD family member 2 (SMAD2), SMAD family member 3 (SMAD3), SMAD family member 4 (SMAD4), SKI proto-oncogene (SKI), SKI-like proto-oncogene (SKIL), TGFB induced factor homeobox 1 (TGIF1), heme oxygenase 2 (HMOX2), interleukin 6 receptor (IL6R), interleukin 6 signal transducer (IL6ST), c-src tyrosine kinase (CSK), phosphoprotein membrane anchor with glycosphingolipid microdomains 1 (PAG1), signaling threshold regulating transmembrane adaptor 1 (SIT1), forkhead box P3 (FOXP3), PR domain 1 (PRDM1), basic leucine zipper transcription factor, ATF-like (BATF), guanylate cyclase 1, soluble, alpha 2 (GUCY1A2), guanylate cyclase 1, soluble, alpha 3 (GUCY1A3), guanylate cyclase 1, soluble, beta 2 (GUCY1B2), prolyl hydroxylase domain (PHD1, PHD2, PHD3) family of proteins, or guanylate cyclase 1, soluble, beta 3 (GUCY1B3), egl-9 family hypoxia-inducible factor 1 (EGLN1), egl-9 family hypoxia-inducible factor 2 (EGLN2), egl-9 family hypoxia-inducible factor 3 (EGLN3), protein phosphatase 1 regulatory subunit 12C (PPP1R12C), NAD-dependent deacetylase sirtuin 2 (SIRT2), and Protein Tyrosine Phosphatase Non-Receptor Type 1 (PTPN1).

In some embodiments, said cancer cells express at least one exogenous protein. In some embodiments, said exogenous protein is a cell surface receptor. In some embodiments, said exogenous protein is an intracellular protein. In some embodiments, a transgene encoding said exogenous protein is integrated into the genome of said cancer cells. In some embodiments, said exogenous protein modulates the ability of an immune cell to recognize and/or kill said cancer cells.

In some embodiments, each of said separate populations of immune cells are contained with separate compartments of one or more arrays.

In one aspect, provided herein are compositions comprising a plurality of separate populations of immune cells, wherein each separate population of immune cells comprises a plurality of immune cells that i) express an exogenous cellular receptor; and ii) comprise a CRISPR system that comprises a guide nucleic acid that binds a portion of a single candidate gene, wherein said single candidate gene is different for each of said separate populations of immune cells; and an exogenous nuclease, or a nucleic acid encoding said exogenous nuclease.

In some embodiments, said population of said plurality of immune cells of each separate population comprises a genomic disruption in said single candidate gene. In some embodiments, at least 70%, 80%, or 90% of said plurality of immune cells of each separate population comprises a genomic disruption in said single candidate gene. In some embodiments, each of said separate populations of immune cells are contained with separate compartments of one or more arrays. In some embodiments, said plurality of separate populations of immune cells comprises at least 10, 20, 30, 40, 50, 60, 70, 80, 90, 100, 200, 300, 400, 500, 600, 700, 800, 900, 1000, 5000, 10000, 50000, or 100000 separate populations of immune cells.

In one aspect, provided herein are compositions comprising a plurality of separate cell populations that each comprise i) a plurality of immune cells that express an exogenous cellular receptor and ii) cells that express a cognate antigen of said exogenous cellular receptor; wherein each of said plurality of immune cells comprises an altered genome sequence of a single candidate gene, and wherein said single candidate gene is different for each of said separate cell populations.

In some embodiments, at least 70%, 80%, or 90% of said plurality of immune cells of each separate cell population comprises said altered genome sequence of said single candidate gene. In some embodiments, each of said separate cell populations are contained with separate compartments of one or more arrays. In some embodiments, said plurality of separate cell populations comprises at least 10, 20, 30, 40, 50, 60, 70, 80, 90, 100, 200, 300, 400, 500, 600, 700, 800, 900, 1000, 5000, 10000, 50000, or 100000 separate cell populations.

In one aspect, provided herein are methods of screening a plurality of single candidate genes, said method comprising: a) expressing an exogenous T-cell receptor (TCR), or a functional fragment thereof, in a plurality of separate populations of T cells, wherein each population comprises a plurality of T cells; b) introducing into each of said separate populations of immune cells a CRISPR system that comprises: i) a guide nucleic acid that binds a portion of a single candidate gene, wherein said single candidate gene is different for each of said separate populations of immune cells; and ii) an exogenous nuclease, or a nucleic acid encoding said exogenous nuclease; thereby generating a plurality of separate populations of engineered T cells that comprise a genomic disruption in said single candidate gene, wherein said genomic disruption suppresses expression of said single candidate gene; c) performing an in vitro assay that comprises contacting said plurality of engineered T cells with a plurality of cells expressing a cognate antigen of said exogenous cellular receptor or said functional fragment thereof in vitro; d) determining a readout of said in vitro assay to thereby determine an effect of said genomic disruption that suppresses expression of said single candidate gene on said plurality of separate populations of engineered T cells; and e) processing said readout to identify a candidate immunomodulatory gene.

In some embodiments, said readout comprises determining a level of cytolytic activity of each of said plurality of separate populations of engineered T cells. In some embodiments, said level of cytolytic activity is determined by a chromium release assay, an electrical impedance assay, time-lapse microscopy, or a co-culture assay.

In some embodiments, said readout comprises determining a level of proliferation of each of said plurality of separate populations of engineered T cells. In some embodiments, said level of proliferation is determined by a Carboxyfluorescein Succinimidyl Ester (CFSE) assay, microscopy, an electrical impedance assay, or flow cytometry.

In some embodiments, said readout comprises determining a level of a factor expressed by each of said plurality of separate populations of engineered T cells. In some embodiments, said factor is a protein. In some embodiments, said protein is secreted from said population of engineered T cells. In some embodiments, said protein is a cytokine or chemokine. In some embodiments, said protein is IL-2, IFNγ, TNFα, LT-α, IL-4, IL-5, IL-6, IL-13, IL-9, IL-10, IL-17A, IL-17F, IL-21, IL-22, IL-26, TNF, CCL20, IL-21, or TGF-β. In some embodiments, said protein is a cell surface protein. In some embodiments, said protein is CD3, CD4, CD8, CD28, CXCR3, CXCR4, CXCR5, CCR6, or CD25. In some embodiments, said protein is CISH, PD1, CTLA4, adenosine A2a receptor (ADORA), CD276, V-set domain containing T cell activation inhibitor 1 (VTCN1), B and T lymphocyte associated (BTLA), indoleamine 2,3-dioxygenase 1 (IDO1), killer cell immunoglobulin-like receptor, three domains, long cytoplasmic tail, 1 (KIR3DL1), lymphocyte-activation gene 3 (LAG3), hepatitis A virus cellular receptor 2 (HAVCR2), V-domain immunoglobulin suppressor of T-cell activation (VISTA), natural killer cell receptor 2B4 (CD244), hypoxanthine phosphoribosyltransferase 1 (HPRT), adeno-associated virus integration site 1 (AAVS1), or chemokine (C—C motif) receptor 5 (gene/pseudogene) (CCR5), CD160 molecule (CD160), T-cell immunoreceptor with Ig and ITIM domains (TIGIT), CD96 molecule (CD96), cytotoxic and regulatory T-cell molecule (CRTAM), leukocyte associated immunoglobulin like receptor 1 (LAIR1), sialic acid binding Ig like lectin 7 (SIGLEC7), sialic acid binding Ig like lectin 9 (SIGLEC9), tumor necrosis factor receptor superfamily member 10b (TNFRSF10B), tumor necrosis factor receptor superfamily member 10a (TNFRSF10A), caspase 8 (CASP8), caspase 10 (CASP10), caspase 3 (CASP3), caspase 6 (CASP6), caspase 7 (CASP7), Fas associated via death domain (FADD), Fas cell surface death receptor (FAS), transforming growth factor beta receptor II (TGFBRII), transforming growth factor beta receptor I (TGFBR1), SMAD family member 2 (SMAD2), SMAD family member 3 (SMAD3), SMAD family member 4 (SMAD4), SKI proto-oncogene (SKI), SKI-like proto-oncogene (SKIL), TGFB induced factor homeobox 1 (TGIF1), programmed cell death 1 (PD-1), cytotoxic T-lymphocyte-associated protein 4 (CTLA4), interleukin 10 receptor subunit alpha (IL10RA), interleukin 10 receptor subunit beta (IL10RB), heme oxygenase 2 (HMOX2), interleukin 6 receptor (IL6R), interleukin 6 signal transducer (IL6ST), c-src tyrosine kinase (CSK), phosphoprotein membrane anchor with glycosphingolipid microdomains 1 (PAG1), signaling threshold regulating transmembrane adaptor 1 (SIT1), forkhead box P3 (FOXP3), PR domain 1 (PRDM1), basic leucine zipper transcription factor, ATF-like (BATF), guanylate cyclase 1, soluble, alpha 2 (GUCY1A2), guanylate cyclase 1, soluble, alpha 3 (GUCY1A3), guanylate cyclase 1, soluble, beta 2 (GUCY1B2), prolyl hydroxylase domain (PHD1, PHD2, PHD3) family of proteins, or guanylate cyclase 1, soluble, beta 3 (GUCY1B3), egl-9 family hypoxia-inducible factor 1 (EGLN1), egl-9 family hypoxia-inducible factor 2 (EGLN2), egl-9 family hypoxia-inducible factor 3 (EGLN3), protein phosphatase 1 regulatory subunit 12C (PPP1R12C), NAD-dependent deacetylase sirtuin 2 (SIRT2), or Protein Tyrosine Phosphatase Non-Receptor Type 1 (PTPN1). In some embodiments, said expression is determined by flow cytometry, western blot, or ELISA.

In some embodiments, at least 60%, 65%, 70%, 75%, 80%, 85%, 90%, 95%, or 99% of T cells of each of said separate populations of T cells comprise said genomic disruption, in the absence of a selection step. In some embodiments, at least 80% of T cells of each of said separate populations of T cells comprise said genomic disruption, in the absence of a selection step. In some embodiments, at least 90% of T cells of each of said separate populations of T cells comprise said genomic disruption, in the absence of a selection step. In some embodiments, said percentage of T cells of each of said separate populations of T cells is determined by Tracking of Indels by Decomposition (TIDE) analysis.

In some embodiments, said exogenous T cell receptor (TCR) is integrated into the genome of said plurality of separate populations of immune cells.

In some embodiments, said exogenous T cell receptor (TCR) is integrated into an endogenous gene sequence that encodes an endogenous T cell receptor. In some embodiments, said gene is TRAC or TCRB.

In some embodiments, said exogenous T cell receptor (TCR) is integrated into a safe harbor site. In some embodiments, said safe harbor site is an AAVS site (e.g., AAVS1, AAVS2), CCR5, or hROSA26.

In some embodiments, said exogenous T cell receptor (TCR) is integrated into a portion of a gene that encodes a protein that functions as a negative regulator of an immune response of said plurality of immune cells. In some embodiments, said gene encodes CISH, PD1, CTLA4, adenosine A2a receptor (ADORA), CD276, V-set domain containing T cell activation inhibitor 1 (VTCN1), B and T lymphocyte associated (BTLA), indoleamine 2,3-dioxygenase 1 (IDO1), killer cell immunoglobulin-like receptor, three domains, long cytoplasmic tail, 1 (KIR3DL1), lymphocyte-activation gene 3 (LAG3), hepatitis A virus cellular receptor 2 (HAVCR2), V-domain immunoglobulin suppressor of T-cell activation (VISTA), natural killer cell receptor 2B4 (CD244), hypoxanthine phosphoribosyltransferase 1 (HPRT), adeno-associated virus integration site 1 (AAVS1), or chemokine (C—C motif) receptor 5 (gene/pseudogene) (CCR5), CD160 molecule (CD160), T-cell immunoreceptor with Ig and ITIM domains (TIGIT), CD96 molecule (CD96), cytotoxic and regulatory T-cell molecule (CRTAM), leukocyte associated immunoglobulin like receptor 1 (LAIR1), sialic acid binding Ig like lectin 7 (SIGLEC7), sialic acid binding Ig like lectin 9 (SIGLEC9), tumor necrosis factor receptor superfamily member 10b (TNFRSF10B), tumor necrosis factor receptor superfamily member 10a (TNFRSF10A), caspase 8 (CASP8), caspase 10 (CASP10), caspase 3 (CASP3), caspase 6 (CASP6), caspase 7 (CASP7), Fas associated via death domain (FADD), Fas cell surface death receptor (FAS), transforming growth factor beta receptor II (TGFBRII), transforming growth factor beta receptor I (TGFBR1), SMAD family member 2 (SMAD2), SMAD family member 3 (SMAD3), SMAD family member 4 (SMAD4), SKI proto-oncogene (SKI), SKI-like proto-oncogene (SKIL), TGFB induced factor homeobox 1 (TGIF1), programmed cell death 1 (PD-1), cytotoxic T-lymphocyte-associated protein 4 (CTLA4), interleukin 10 receptor subunit alpha (IL10RA), interleukin 10 receptor subunit beta (IL10RB), heme oxygenase 2 (HMOX2), interleukin 6 receptor (IL6R), interleukin 6 signal transducer (IL6ST), c-src tyrosine kinase (CSK), phosphoprotein membrane anchor with glycosphingolipid microdomains 1 (PAG1), signaling threshold regulating transmembrane adaptor 1 (SIT1), forkhead box P3 (FOXP3), PR domain 1 (PRDM1), basic leucine zipper transcription factor, ATF-like (BATF), guanylate cyclase 1, soluble, alpha 2 (GUCY1A2), guanylate cyclase 1, soluble, alpha 3 (GUCY1A3), guanylate cyclase 1, soluble, beta 2 (GUCY1B2), prolyl hydroxylase domain (PHD1, PHD2, PHD3) family of proteins, or guanylate cyclase 1, soluble, beta 3 (GUCY1B3), egl-9 family hypoxia-inducible factor 1 (EGLN1), egl-9 family hypoxia-inducible factor 2 (EGLN2), egl-9 family hypoxia-inducible factor 3 (EGLN3), protein phosphatase 1 regulatory subunit 12C (PPP1R12C), NAD-dependent deacetylase sirtuin 2 (SIRT2), or Protein Tyrosine Phosphatase Non-Receptor Type 1 (PTPN1).

In some embodiments, at least 50%, 60%, 65%, 70%, 75%, 80%, 85%, 90%, 95%, or 99% of T cells of each of said separate populations of T cells express said exogenous T cell receptor, in the absence of a selection step. In some embodiments, at least 70% of immune cells of each of said separate populations of T cells express said exogenous T cell receptor, in the absence of a selection step. In some embodiments, at least 80% of immune cells of each of said separate populations of T cells express said exogenous T cell receptor, in the absence of a selection step. In some embodiments, at least 90% of immune cells of each of said separate populations of T cells express said exogenous T cell receptor, in the absence of a selection step. In some embodiments, said percentage of T cells of each of said separate populations of immune cells is determined by flow cytometry or sequencing.

In some embodiments, said genomic disruption is a double strand break. In some embodiments, said nuclease is introduced using electroporation. In some embodiments, said nuclease is an endonuclease. In some embodiments, said endonuclease is selected from the group consisting of Cas1, Cas1B, Cas2, Cas3, Cas4, Cas5, Cas6, Cas7, Cas8, Cas9, Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx1S, Csf1, Csf2, CsO, Csf4, Cpf1, c2c1, c2c3, and Cas9HiFi. In some embodiments, said endonuclease is Cas9.

In some embodiments, said guide nucleic acid is a guide ribonucleic acid (gRNA). In some embodiments, said guide nucleic acid comprises a phosphorothioate (PS) linkage, a 2′-fluoro (2′-F) modification, a 2′-O-methyl (2′-O-Me) linkage, a 2-O-Methyl 3phosphorothioate linkage, a S-constrained ethyl (cEt) modification, or any combination thereof. In some embodiments, said guide nucleic acid is introduced using electroporation.

In some embodiments, said exogenous T cell receptor (TCR) is introduced using electroporation. In some embodiments, said exogenous T cell receptor (TCR) is introduced using a viral vector. In some embodiments, said viral vector is an adeno-associated virus (AAV) vector. In some embodiments, said AAV vector is selected from the group consisting of a recombinant AAV (rAAV) vector, a hybrid AAV vector, a chimeric AAV vector, a self-complementary AAV (scAAV) vector, a modified AAV vector, and any combination thereof. In some embodiments, said AAV vector is a chimeric AAV vector. In some embodiments, said chimeric AAV vector comprises a modification in at least one AAV capsid gene sequence.

In some embodiments, said single gene is an immunomodulatory gene. In some embodiments, said single gene is a candidate immune checkpoint gene.

In some embodiments, said method further comprises cryopreserving said separate populations of engineered T cells.

In some embodiments, said method further comprises processing said readout to identify a candidate immunomodulatory gene. In some embodiments, said processing comprises determining a criterion from at least one of: cytolytic activity, gene expression of said candidate immunomodulatory gene, intracellular location of a protein encoded by said candidate immunomodulatory gene, loss-of-function association with a human disease of said candidate immunomodulatory gene, a guide nucleic acid score of a guide nucleic acid that binds to a portion of said candidate immunomodulatory gene, existing drugs in development that target said candidate immunomodulatory gene, existing drugs that target said candidate immunomodulatory gene, or loss-of-function phenotype of said candidate immunomodulatory gene, or any combination thereof.

In some embodiments, said processing comprises determining a criterion from at least two, three, four, five, six, seven, or eight of: cytolytic activity, gene expression of said candidate immunomodulatory gene, intracellular location of a protein encoded by said candidate immunomodulatory gene, loss-of-function association with a human disease of said candidate immunomodulatory gene, a guide nucleic acid score of a guide nucleic acid that binds to a portion of said candidate immunomodulatory gene, existing drugs in development that target said candidate immunomodulatory gene, existing drugs that target said candidate immunomodulatory gene, or loss-of-function phenotype of said candidate immunomodulatory gene, or any combination thereof.

In some embodiments, said processing comprises ranking at least two candidate immunomodulatory genes according to said at least one criterion to produce ranked candidate immunomodulatory genes. In some embodiments, said processing comprises ranking at least two candidate immunomodulatory genes according to said at least two, three, four, five, six, seven, or eight criterion to produce ranked candidate immunomodulatory genes. In some embodiments, said processing comprises ranking at least 10, 100, 200, 300, 400, 500, 600, 700, 800, 900, 1000, 5000, 10000, 50000, or 100000 candidate immunomodulatory genes according to said at least one criterion. In some embodiments, said processing comprises ranking at least 10, 100, 200, 300, 400, 500, 600, 700, 800, 900, 1000, 5000, 10000, 50000, or 100000 candidate immunomodulatory genes according to said at least two, three, four, five, six, seven, or eight criterion.

In some embodiments, said method further comprises selecting a top 10, 20, 30, 40, or 50 of said ranked candidate immunomodulatory genes to thereby generate a ranked output. In some embodiments, said method further comprises identifying at least one of a gene family, a gene function, or an intracellular signaling pathway from said ranked output, to thereby generate an analyzed ranked output. In some embodiments, said method further comprises correlating cytolytic activity of said analyzed ranked output, to thereby generate a cytolytic-correlated ranked output. In some embodiments, said method further comprises ranking said candidate immunomodulatory genes from said cytolytic-correlated ranked output according to said intracellular location of a protein encoded by said candidate immunomodulatory gene. In some embodiments, said method further comprises ranking said candidate immunomodulatory genes from said cytolytic-correlated ranked output according to said existing drug in development that targets said candidate immunomodulatory gene and said existing drug against said candidate immunomodulatory gene.

In some embodiments, each of said separate populations of engineered T cells comprises a plurality of CD8+ T cells. In some embodiments, each of said separate populations of engineered T cells comprises a plurality of CD4+ T cells. In some embodiments, each of said separate populations of engineered T cells comprises a plurality of CD4+ T cells and CD8+ T cells. In some embodiments, each of said separate populations of engineered T cells comprises tumor infiltrating T cells (TILs). In some embodiments, each of said separate populations of engineered T cells comprises a plurality of human cells. In some embodiments, each of said separate populations of engineered T cells comprises a plurality of primary cells. In some embodiments, each of said separate populations of engineered T cells comprises a plurality of ex vivo cells.

In some embodiments, each of said separate populations of T cells comprises at least 10, 20, 30, 40, 50, 60, 70, 80, 90, 100, 200, 300, 400, 500, 600, 700, 800, 900, 1000, 5000, 10000, 50000, or 100000 separate populations of T cells.

In some embodiments, said each of said separate populations of engineered T cells comprises a transgene that encodes for a protein that improves immunomodulatory function of said engineered T cells. In some embodiments, said protein is phosphodiesterase 1C (PDE1C), rhotekin 2 (RTKN2), nerve growth factor receptor (NGFR), or thymocyte-expressed molecule involved in selection (THEMIS).

In some embodiments, said transgene is integrated into a safe harbor site. In some embodiments, said safe harbor site is site is an AAVS site (e.g., AAVS1, AAVS2), CCR5, or hROSA26.

In some embodiments, said transgene is integrated into a portion of a gene that encodes a protein that functions as a negative regulator of an immune response of said plurality of T cells. In some embodiments, said integration decreases or inhibits expression of a functional version of said protein that functions as a negative regulator of an immune response. In some embodiments, said protein is CISH, PD1, CTLA4, adenosine A2a receptor (ADORA), CD276, V-set domain containing T cell activation inhibitor 1 (VTCN1), B and T lymphocyte associated (BTLA), indoleamine 2,3-dioxygenase 1 (IDO1), killer cell immunoglobulin-like receptor, three domains, long cytoplasmic tail, 1 (KIR3DL1), lymphocyte-activation gene 3 (LAG3), hepatitis A virus cellular receptor 2 (HAVCR2), V-domain immunoglobulin suppressor of T-cell activation (VISTA), natural killer cell receptor 2B4 (CD244), hypoxanthine phosphoribosyltransferase 1 (HPRT), adeno-associated virus integration site 1 (AAVS1), or chemokine (C—C motif) receptor 5 (gene/pseudogene) (CCR5), CD160 molecule (CD160), T-cell immunoreceptor with Ig and ITIM domains (TIGIT), CD96 molecule (CD96), cytotoxic and regulatory T-cell molecule (CRTAM), leukocyte associated immunoglobulin like receptor 1 (LAIR1), sialic acid binding Ig like lectin 7 (SIGLEC7), sialic acid binding Ig like lectin 9 (SIGLEC9), tumor necrosis factor receptor superfamily member 10b (TNFRSF10B), tumor necrosis factor receptor superfamily member 10a (TNFRSF10A), caspase 8 (CASP8), caspase 10 (CASP10), caspase 3 (CASP3), caspase 6 (CASP6), caspase 7 (CASP7), Fas associated via death domain (FADD), Fas cell surface death receptor (FAS), transforming growth factor beta receptor II (TGFBRII), transforming growth factor beta receptor I (TGFBR1), SMAD family member 2 (SMAD2), SMAD family member 3 (SMAD3), SMAD family member 4 (SMAD4), SKI proto-oncogene (SKI), SKI-like proto-oncogene (SKIL), TGFB induced factor homeobox 1 (TGIF1), programmed cell death 1 (PD-1), cytotoxic T-lymphocyte-associated protein 4 (CTLA4), interleukin 10 receptor subunit alpha (IL10RA), interleukin 10 receptor subunit beta (IL10RB), heme oxygenase 2 (HMOX2), interleukin 6 receptor (IL6R), interleukin 6 signal transducer (IL6ST), c-src tyrosine kinase (CSK), phosphoprotein membrane anchor with glycosphingolipid microdomains 1 (PAG1), signaling threshold regulating transmembrane adaptor 1 (SIT1), forkhead box P3 (FOXP3), PR domain 1 (PRDM1), basic leucine zipper transcription factor, ATF-like (BATF), guanylate cyclase 1, soluble, alpha 2 (GUCY1A2), guanylate cyclase 1, soluble, alpha 3 (GUCY1A3), guanylate cyclase 1, soluble, beta 2 (GUCY1B2), prolyl hydroxylase domain (PHD1, PHD2, PHD3) family of proteins, or guanylate cyclase 1, soluble, beta 3 (GUCY1B3), egl-9 family hypoxia-inducible factor 1 (EGLN1), egl-9 family hypoxia-inducible factor 2 (EGLN2), egl-9 family hypoxia-inducible factor 3 (EGLN3), protein phosphatase 1 regulatory subunit 12C (PPP1R12C), NAD-dependent deacetylase sirtuin 2 (SIRT2), or Protein Tyrosine Phosphatase Non-Receptor Type 1 (PTPN1).

In some embodiments, said each of said populations of engineered T cells comprises a genetic modification that enhances expression of a gene that encodes for a protein that improves immunomodulatory function of said engineered T cells. In some embodiments, said protein is phosphodiesterase 1C (PDE1C), rhotekin 2 (RTKN2), nerve growth factor receptor (NGFR), or thymocyte-expressed molecule involved in selection (THEMIS).

In some embodiments, said method further comprises selecting T cells that express said exogenous TCR or functional fragment thereof.

In some embodiments, said plurality of cells that express said cognate antigen are cancer cells. In some embodiments, said cancer cells are primary cancer cells or from a cancer cell line. In some embodiments, said cancer cells comprise a genomic disruption in at least one gene. In some embodiments, said genomic disruption is mediated by a CRISPR system that comprises a gRNA that binds to a portion of said gene and a nuclease that mediates cleavage of genomic DNA. In some embodiments, said genomic disruption is a double strand break. In some embodiments, said at least one gene encodes a protein that that a negative regulator of an immune response. In some embodiments, said protein is a ligand of a checkpoint inhibitor. In some embodiments, said protein is a ligand of a checkpoint inhibitor selected from the group consisting of programmed cell death 1 (PD-1), cytotoxic T-lymphocyte-associated protein 4 (CTLA4), interleukin 10 receptor subunit alpha (IL10RA), interleukin 10 receptor subunit beta (IL10RB), adenosine A2a receptor (ADORA), CD276, V-set domain containing T cell activation inhibitor 1 (VTCN1), B and T lymphocyte associated (BTLA), indoleamine 2,3-dioxygenase 1 (IDO1), killer cell immunoglobulin-like receptor, three domains, long cytoplasmic tail, 1 (KIR3DL1), lymphocyte-activation gene 3 (LAG3), hepatitis A virus cellular receptor 2 (HAVCR2), V-domain immunoglobulin suppressor of T-cell activation (VISTA), natural killer cell receptor 2B4 (CD244), hypoxanthine phosphoribosyltransferase 1 (HPRT), adeno-associated virus integration site 1 (AAVS1), or chemokine (C—C motif) receptor 5 (gene/pseudogene) (CCR5), CD160 molecule (CD160), T-cell immunoreceptor with Ig and ITIM domains (TIGIT), CD96 molecule (CD96), cytotoxic and regulatory T-cell molecule (CRTAM), leukocyte associated immunoglobulin like receptor 1 (LAIR1), sialic acid binding Ig like lectin 7 (SIGLEC7), sialic acid binding Ig like lectin 9 (SIGLEC9), tumor necrosis factor receptor superfamily member 10b (TNFRSF10B), tumor necrosis factor receptor superfamily member 10a (TNFRSF10A), caspase 8 (CASP8), caspase 10 (CASP10), caspase 3 (CASP3), caspase 6 (CASP6), caspase 7 (CASP7), Fas associated via death domain (FADD), Fas cell surface death receptor (FAS), transforming growth factor beta receptor II (TGFBRII), transforming growth factor beta receptor I (TGFBR1), SMAD family member 2 (SMAD2), SMAD family member 3 (SMAD3), SMAD family member 4 (SMAD4), SKI proto-oncogene (SKI), SKI-like proto-oncogene (SKIL), TGFB induced factor homeobox 1 (TGIF1), heme oxygenase 2 (HMOX2), interleukin 6 receptor (IL6R), interleukin 6 signal transducer (IL6ST), c-src tyrosine kinase (CSK), phosphoprotein membrane anchor with glycosphingolipid microdomains 1 (PAG1), signaling threshold regulating transmembrane adaptor 1 (SIT1), forkhead box P3 (FOXP3), PR domain 1 (PRDM1), basic leucine zipper transcription factor, ATF-like (BATF), guanylate cyclase 1, soluble, alpha 2 (GUCY1A2), guanylate cyclase 1, soluble, alpha 3 (GUCY1A3), guanylate cyclase 1, soluble, beta 2 (GUCY1B2), prolyl hydroxylase domain (PHD1, PHD2, PHD3) family of proteins, or guanylate cyclase 1, soluble, beta 3 (GUCY1B3), egl-9 family hypoxia-inducible factor 1 (EGLN1), egl-9 family hypoxia-inducible factor 2 (EGLN2), egl-9 family hypoxia-inducible factor 3 (EGLN3), protein phosphatase 1 regulatory subunit 12C (PPP1R12C), NAD-dependent deacetylase sirtuin 2 (SIRT2), and Protein Tyrosine Phosphatase Non-Receptor Type 1 (PTPN1).

In some embodiments, said cancer cells express at least one exogenous protein. In some embodiments, said exogenous protein is a cell surface receptor. In some embodiments, said exogenous protein is an intracellular protein. In some embodiments, a transgene encoding said exogenous protein is integrated into the genome of said cancer cells. In some embodiments, said exogenous protein modulates the ability of an immune cell to recognize and/or kill said cancer cells.

In some embodiments, each of said separate populations of immune cells are contained with separate compartments of one or more arrays.

In one aspect, provided herein are compositions comprising a plurality of separate populations of T cells, wherein each separate population of T cells comprises a plurality of T cells that i) express an exogenous cellular receptor; and ii) comprise a CRISPR system that comprises a guide nucleic acid that binds a portion of a single candidate gene, wherein said single candidate gene is different for each of said separate populations of T cells; and an exogenous nuclease, or a nucleic acid encoding said exogenous nuclease.

In some embodiments, said population of said plurality of T cells of each separate population comprises a genomic disruption in said single candidate gene. In some embodiments, at least 70%, 80%, or 90% of said plurality of T cells of each separate population comprises a genomic disruption in said single candidate gene. In some embodiments, each of said separate populations of T cells are contained with separate compartments of one or more arrays. In some embodiments, said plurality of separate populations of T cells comprises at least 10, 20, 30, 40, 50, 60, 70, 80, 90, 100, 200, 300, 400, 500, 600, 700, 800, 900, 1000, 5000, 10000, 50000, or 100000 separate populations of T cells.

In one aspect, provided herein are compositions comprising a plurality of separate cell populations that each comprise i) a plurality of T cells that express an exogenous cellular receptor and ii) cells that express a cognate antigen of said exogenous cellular receptor; wherein each of said plurality of T cells comprises an altered genome sequence of a single candidate gene, and wherein said single candidate gene is different for each of said separate cell populations.

In some embodiments, at least 70%, 80%, or 90% of said plurality of T cells of each separate cell population comprises said altered genome sequence of said single candidate gene. In some embodiments, each of said separate cell populations are contained with separate compartments of one or more arrays. In some embodiments, said plurality of separate cell populations comprises at least 10, 20, 30, 40, 50, 60, 70, 80, 90, 100, 200, 300, 400, 500, 600, 700, 800, 900, 1000, 5000, 10000, 50000, or 100000 separate cell populations.

In one aspect, provided herein are methods of screening a plurality of single candidate genes, said method comprising: a) obtaining a plurality of separate populations of cancer cells that express an antigen, wherein each population comprises a plurality of cancer cells; b) introducing into each of said separate populations of cancer cells a CRISPR system that comprises: i) a guide nucleic acid that binds a portion of a single candidate gene, wherein said single candidate gene is different for each of said separate populations of cancer cells; and ii) an exogenous nuclease, or a nucleic acid encoding said exogenous nuclease; thereby generating a plurality of separate populations of engineered cancer cells that comprise a genomic disruption in said single candidate gene, wherein said genomic disruption suppresses expression of said single candidate gene; c) performing an in vitro assay that comprises contacting in vitro said plurality of engineered cancer cells with a plurality of immune cells that express a cellular receptor, or functional fragment thereof, that binds to said antigen; and d) obtaining a readout from said in vitro assay, to thereby determine an effect of said genomic disruption that suppresses expression of said single candidate gene on said plurality of separate populations of engineered cancer cells or said immune cells that express a cellular receptor, or functional fragment thereof, that binds to said antigen.

In some embodiments, said readout comprises determining a level of cell death of each of said separate populations of engineered cancer cells. In some embodiments, said level of cell death is determined by flow cytometry or microscopy.

In some embodiments, said readout comprises determining a time to which a certain percentage of cells each of said separate populations of engineered cancer cells are killed. In some embodiments, said level of cell death is determined by flow cytometry or microscopy.

In some embodiments, said readout comprises determining a level of cytolytic activity of said plurality of immune cells. In some embodiments, said level of cytolytic activity is determined by a chromium release assay, an electrical impedance assay, time-lapse microscopy, or a co-culture assay.

In some embodiments, said readout comprises determining a level of proliferation of said plurality of immune cells. In some embodiments, said level of proliferation is determined by a Carboxyfluorescein Succinimidyl Ester (CFSE) assay, microscopy, an electrical impedance assay, or flow cytometry.

In some embodiments, said readout comprises determining a level of a factor expressed by said plurality of immune cells. In some embodiments, said factor is a protein. In some embodiments, said protein is secreted from said population of engineered immune cells. In some embodiments, said protein is a cytokine or chemokine. In some embodiments, said protein is a cell surface protein. In some embodiments, said expression is determined by flow cytometry, western blot, or ELISA.

In some embodiments, said antigen is an endogenous antigen. In some embodiments, said antigen is an exogenous antigen. The method of claim 111, wherein step a. comprises expressing said exogenous antigen in each of said separate populations of cancer cells.

In some embodiments, at least 60%, 65%, 70%, 75%, 80%, 85%, 90%, 95%, or 99% of cancer cells of each of said separate populations of cancer cells comprise said genomic disruption, in the absence of a selection step. In some embodiments, at least 80% of cancer cells of each of said separate populations of cancer cells comprise said genomic disruption, in the absence of a selection step. In some embodiments, at least 90% of cancer cells of each of said separate populations of cancer cells comprise said genomic disruption, in the absence of a selection step. In some embodiments, said percentage of cancer cells of each of said separate populations of cancer cells is determined by Tracking of Indels by Decomposition (TIDE) analysis.

In some embodiments, said cellular receptor is an immunomodulatory cellular receptor. In some embodiments, said cellular receptor is an exogenous cellular receptor. In some embodiments, said exogenous cellular receptor is integrated into the genome of said plurality of immune cells.

In some embodiments, said exogenous cellular receptor is integrated into an endogenous gene sequence that encodes an exogenous cellular receptor. In some embodiments, said exogenous cellular receptor is integrated into a safe harbor site. In some embodiments, said safe harbor site is site is an AAVS site (e.g., AAVS1, AAVS2), CCR5, or hROSA26. In some embodiments, said exogenous cellular receptor is integrated into a portion of a gene that encodes a protein that functions as a negative regulator of an immune response of said plurality of immune cells. In some embodiments, said integration decreases or inhibits expression of said protein that functions as a negative regulator of an immune response of said plurality of immune cells. In some embodiments, said gene encodes for a protein selected from the group consisting of CISH, PD1, CTLA4, adenosine A2a receptor (ADORA), CD276, V-set domain containing T cell activation inhibitor 1 (VTCN1), B and T lymphocyte associated (BTLA), indoleamine 2,3-dioxygenase 1 (IDO1), killer cell immunoglobulin-like receptor, three domains, long cytoplasmic tail, 1 (KIR3DL1), lymphocyte-activation gene 3 (LAG3), hepatitis A virus cellular receptor 2 (HAVCR2), V-domain immunoglobulin suppressor of T-cell activation (VISTA), natural killer cell receptor 2B4 (CD244), hypoxanthine phosphoribosyltransferase 1 (HPRT), adeno-associated virus integration site 1 (AAVS1), or chemokine (C—C motif) receptor 5 (gene/pseudogene) (CCR5), CD160 molecule (CD160), T-cell immunoreceptor with Ig and ITIM domains (TIGIT), CD96 molecule (CD96), cytotoxic and regulatory T-cell molecule (CRTAM), leukocyte associated immunoglobulin like receptor 1 (LAIR1), sialic acid binding Ig like lectin 7 (SIGLEC7), sialic acid binding Ig like lectin 9 (SIGLEC9), tumor necrosis factor receptor superfamily member 10b (TNFRSF10B), tumor necrosis factor receptor superfamily member 10a (TNFRSF10A), caspase 8 (CASP8), caspase 10 (CASP10), caspase 3 (CASP3), caspase 6 (CASP6), caspase 7 (CASP7), Fas associated via death domain (FADD), Fas cell surface death receptor (FAS), transforming growth factor beta receptor II (TGFBRII), transforming growth factor beta receptor I (TGFBR1), SMAD family member 2 (SMAD2), SMAD family member 3 (SMAD3), SMAD family member 4 (SMAD4), SKI proto-oncogene (SKI), SKI-like proto-oncogene (SKIL), TGFB induced factor homeobox 1 (TGIF1), programmed cell death 1 (PD-1), cytotoxic T-lymphocyte-associated protein 4 (CTLA4), interleukin 10 receptor subunit alpha (IL10RA), interleukin 10 receptor subunit beta (IL10RB), heme oxygenase 2 (HMOX2), interleukin 6 receptor (IL6R), interleukin 6 signal transducer (IL6ST), c-src tyrosine kinase (CSK), phosphoprotein membrane anchor with glycosphingolipid microdomains 1 (PAG1), signaling threshold regulating transmembrane adaptor 1 (SIT1), forkhead box P3 (FOXP3), PR domain 1 (PRDM1), basic leucine zipper transcription factor, ATF-like (BATF), guanylate cyclase 1, soluble, alpha 2 (GUCY1A2), guanylate cyclase 1, soluble, alpha 3 (GUCY1A3), guanylate cyclase 1, soluble, beta 2 (GUCY1B2), prolyl hydroxylase domain (PHD1, PHD2, PHD3) family of proteins, or guanylate cyclase 1, soluble, beta 3 (GUCY1B3), egl-9 family hypoxia-inducible factor 1 (EGLN1), egl-9 family hypoxia-inducible factor 2 (EGLN2), egl-9 family hypoxia-inducible factor 3 (EGLN3), protein phosphatase 1 regulatory subunit 12C (PPP1R12C), NAD-dependent deacetylase sirtuin 2 (SIRT2), or Protein Tyrosine Phosphatase Non-Receptor Type 1 (PTPN1).

In some embodiments, at least 50%, 60%, 65%, 70%, 75%, 80%, 85%, 90%, 95%, or 99% of said plurality of immune cells express said cellular receptor, in the absence of a selection step. In some embodiments, said percentage of immune cells of said plurality of immune cells is determined by flow cytometry or sequencing.

In some embodiments, said genomic disruption is a double strand break. In some embodiments, said nuclease is introduced using electroporation. In some embodiments, said nuclease is an endonuclease. In some embodiments, said endonuclease is selected from the group consisting of Cas1, Cas1B, Cas2, Cas3, Cas4, Cas5, Cas6, Cas7, Cas8, Cas9, Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx1S, Csf1, Csf2, CsO, Csf4, Cpf1, c2c1, c2c3, and Cas9HiFi. In some embodiments, said endonuclease is Cas9.

In some embodiments, said guide nucleic acid is a guide ribonucleic acid (gRNA).

In some embodiments, said guide nucleic acid comprises a phosphorothioate (PS) linkage, a 2′-fluoro (2′-F) modification, a 2′-O-methyl (2′-O-Me) linkage, a 2-O-Methyl 3phosphorothioate linkage, a S-constrained ethyl (cEt) modification, or any combination thereof. In some embodiments, said guide nucleic acid is introduced using electroporation.

In some embodiments, said cellular receptor is an exogenous cellular receptor introduced using electroporation. In some embodiments, said cellular receptor is an exogenous cellular receptor introduced using a viral vector.

In some embodiments, said viral vector is an adeno-associated virus (AAV) vector. In some embodiments, said AAV vector is selected from the group consisting of a recombinant AAV (rAAV) vector, a hybrid AAV vector, a chimeric AAV vector, a self-complementary AAV (scAAV) vector, a modified AAV vector, and any combination thereof. In some embodiments, said AAV vector is a chimeric AAV vector. In some embodiments, said chimeric AAV vector comprises a modification in at least one AAV capsid gene sequence.

In some embodiments, said cellular receptor is a T-cell receptor (TCR), B cell receptor (BCR), NK cell receptor, dendritic cell receptor, monocyte receptor, macrophage receptor, neutrophil receptor, eosinophil receptor, or a chimeric antigen receptor (CAR). In some embodiments, said cellular receptor is a T-cell receptor (TCR).

In some embodiments, said single gene is an immunomodulatory gene. In some embodiments, said single gene is a candidate immune checkpoint receptor ligand gene.

In some embodiments, said method further comprises cryopreserving said separate populations of engineered cancer cells.

In some embodiments, said method further comprises processing said readout to identify a candidate immunomodulatory gene.

In some embodiments, said processing comprises determining a criterion from at least one of: cytolytic activity, gene expression of said candidate immunomodulatory gene, intracellular location of a protein encoded by said candidate immunomodulatory gene, loss-of-function association with a human disease of said candidate immunomodulatory gene, a guide nucleic acid score of a guide nucleic acid that binds to a portion of said candidate immunomodulatory gene, existing drugs in development that target said candidate immunomodulatory gene, existing drugs that target said candidate immunomodulatory gene, or loss-of-function phenotype of said candidate immunomodulatory gene, or any combination thereof.

In some embodiments, said processing comprises determining a criterion from at least two, three, four, five, six, seven, or eight of: cytolytic activity, gene expression of said candidate immunomodulatory gene, intracellular location of a protein encoded by said candidate immunomodulatory gene, loss-of-function association with a human disease of said candidate immunomodulatory gene, a guide nucleic acid score of a guide nucleic acid that binds to a portion of said candidate immunomodulatory gene, existing drugs in development that target said candidate immunomodulatory gene, existing drugs that target said candidate immunomodulatory gene, or loss-of-function phenotype of said candidate immunomodulatory gene, or any combination thereof.

In some embodiments, said processing comprises ranking at least two candidate immunomodulatory genes according to said at least one criterion to produce ranked candidate immunomodulatory genes. In some embodiments, said processing comprises ranking at least 10, 100, 200, 300, 400, 500, 600, 700, 800, 900, 1000, 5000, 10000, 50000, or 100000 candidate immunomodulatory genes according to said at least one criterion.

In some embodiments, said processing comprises ranking at least two candidate immunomodulatory genes according to said at least two, three, four, five, six, seven, or eight criterion to produce ranked candidate immunomodulatory genes. In some embodiments, said processing comprises ranking at least 10, 100, 200, 300, 400, 500, 600, 700, 800, 900, 1000, 5000, 10000, 50000, or 100000 candidate immunomodulatory genes according to said at least one criterion.

In some embodiments, said processing comprises ranking at least 10, 100, 200, 300, 400, 500, 600, 700, 800, 900, 1000, 5000, 10000, 50000, or 100000 candidate immunomodulatory genes according to said at least two, three, four, five, six, seven, or eight criterion.

In some embodiments, said method further comprises selecting a top 10, 20, 30, 40, or 50 of said ranked candidate immunomodulatory genes to thereby generate a ranked output.

In some embodiments, said method further comprises identifying at least one of a gene family, a gene function, or an intracellular signaling pathway from said ranked output, to thereby generate an analyzed ranked output.

In some embodiments, said method further comprises correlating cytolytic activity of said analyzed ranked output, to thereby generate a cytolytic-correlated ranked output.

In some embodiments, said method further comprises ranking said candidate immunomodulatory genes from said cytolytic-correlated ranked output according to said intracellular location of a protein encoded by said candidate immunomodulatory gene.

In some embodiments, said method further comprises ranking said candidate immunomodulatory genes from said cytolytic-correlated ranked output according to said existing drug in development that targets said candidate immunomodulatory gene and said existing drug against said candidate immunomodulatory gene.

In some embodiments, said plurality of immune cells comprises a plurality of T cells, tumor infiltrating lymphocytes (TILs), NK cells, B cell, dendritic cells, monocytes, macrophages, neutrophils, or eosinophils.

In some embodiments, said plurality of immune cells comprises a plurality of T cells. In some embodiments, said plurality of T cells comprises a plurality of CD8+ T cells. In some embodiments, said plurality of T cells comprises a plurality of CD4+ T cells. In some embodiments, said plurality of T cells comprises a plurality of CD4+ T cells and CD8+ T cells.

The method of any one of claims 95-163, wherein said plurality of immune cells comprises a plurality of human cells. In some embodiments, said plurality of immune cells comprises a plurality of primary cells. In some embodiments, said plurality of immune cells comprises a plurality of ex vivo cells.

In some embodiments, said plurality of separate populations of cancer cells comprises at least 10, 20, 30, 40, 50, 60, 70, 80, 90, 100, 200, 300, 400, 500, 600, 700, 800, 900, 1000, 5000, 10000, 50000, or 100000 separate populations of cancer cells.

In some embodiments, said plurality of immune cells comprises a transgene that encodes for a protein that improves immunomodulatory function of said immune cells. In some embodiments, said transgene is integrated in the genome of said immune cells. In some embodiments, said transgene is integrated into a safe harbor site. In some embodiments, said safe harbor site is site is an AAVS site (e.g., AAVS1, AAVS2), CCR5, or hROSA26. In some embodiments, said plurality of immune cells comprises a genetic modification that enhances expression of a gene that encodes for a protein that improves immunomodulatory function of said immune cells. In some embodiments, said transgene is integrated into a portion of a gene that encodes a protein that functions as a negative regulator of an immune response of said immune cells.

In some embodiments, each of said separate populations of cancer cells comprise a genomic disruption in at least one gene. In some embodiments, said genomic disruption is mediated by a CRISPR system that comprises a gRNA that binds to a portion of said gene and a nuclease that mediates cleavage of genomic DNA. In some embodiments, said genomic disruption is a double strand break. In some embodiments, said at least one gene encodes a protein that that a negative regulator of an immune response. In some embodiments, said protein is a ligand of a checkpoint inhibitor. In some embodiments, said protein is a ligand of a checkpoint inhibitor selected from the group consisting of programmed cell death 1 (PD-1), cytotoxic T-lymphocyte-associated protein 4 (CTLA4), interleukin 10 receptor subunit alpha (IL10RA), interleukin 10 receptor subunit beta (IL10RB), adenosine A2a receptor (ADORA), CD276, V-set domain containing T cell activation inhibitor 1 (VTCN1), B and T lymphocyte associated (BTLA), indoleamine 2,3-dioxygenase 1 (IDO1), killer cell immunoglobulin-like receptor, three domains, long cytoplasmic tail, 1 (KIR3DL1), lymphocyte-activation gene 3 (LAG3), hepatitis A virus cellular receptor 2 (HAVCR2), V-domain immunoglobulin suppressor of T-cell activation (VISTA), natural killer cell receptor 2B4 (CD244), hypoxanthine phosphoribosyltransferase 1 (HPRT), adeno-associated virus integration site 1 (AAVS1), or chemokine (C—C motif) receptor 5 (gene/pseudogene) (CCR5), CD160 molecule (CD160), T-cell immunoreceptor with Ig and ITIM domains (TIGIT), CD96 molecule (CD96), cytotoxic and regulatory T-cell molecule (CRTAM), leukocyte associated immunoglobulin like receptor 1 (LAIR1), sialic acid binding Ig like lectin 7 (SIGLEC7), sialic acid binding Ig like lectin 9 (SIGLEC9), tumor necrosis factor receptor superfamily member 10b (TNFRSF10B), tumor necrosis factor receptor superfamily member 10a (TNFRSF10A), caspase 8 (CASP8), caspase 10 (CASP10), caspase 3 (CASP3), caspase 6 (CASP6), caspase 7 (CASP7), Fas associated via death domain (FADD), Fas cell surface death receptor (FAS), transforming growth factor beta receptor II (TGFBRII), transforming growth factor beta receptor I (TGFBR1), SMAD family member 2 (SMAD2), SMAD family member 3 (SMAD3), SMAD family member 4 (SMAD4), SKI proto-oncogene (SKI), SKI-like proto-oncogene (SKIL), TGFB induced factor homeobox 1 (TGIF1), heme oxygenase 2 (HMOX2), interleukin 6 receptor (IL6R), interleukin 6 signal transducer (IL6ST), c-src tyrosine kinase (CSK), phosphoprotein membrane anchor with glycosphingolipid microdomains 1 (PAG1), signaling threshold regulating transmembrane adaptor 1 (SIT1), forkhead box P3 (FOXP3), PR domain 1 (PRDM1), basic leucine zipper transcription factor, ATF-like (BATF), guanylate cyclase 1, soluble, alpha 2 (GUCY1A2), guanylate cyclase 1, soluble, alpha 3 (GUCY1A3), guanylate cyclase 1, soluble, beta 2 (GUCY1B2), prolyl hydroxylase domain (PHD1, PHD2, PHD3) family of proteins, or guanylate cyclase 1, soluble, beta 3 (GUCY1B3), egl-9 family hypoxia-inducible factor 1 (EGLN1), egl-9 family hypoxia-inducible factor 2 (EGLN2), egl-9 family hypoxia-inducible factor 3 (EGLN3), protein phosphatase 1 regulatory subunit 12C (PPP1R12C), NAD-dependent deacetylase sirtuin 2 (SIRT2), and Protein Tyrosine Phosphatase Non-Receptor Type 1 (PTPN1).

In some embodiments, said cancer cells express at least one exogenous protein. In some embodiments, said exogenous protein is a cell surface receptor. In some embodiments, said exogenous protein is an intracellular protein. In some embodiments, a transgene encoding said exogenous protein is integrated into the genome of said cancer cells. In some embodiments, said exogenous protein modulates the ability of an immune cell to recognize and/or kill said cancer cells.

In some embodiments, each of said separate populations of immune cells are contained with separate compartments of one or more arrays.

In one aspect, provided herein are compositions comprising a plurality of separate populations of cancer cells, wherein each separate population of cancer cells comprises a plurality of cancer cells that i) expresses an antigen; and ii); comprise a CRISPR system that comprises a guide nucleic acid that binds a portion of a single candidate gene, wherein said single candidate gene is different for each of said separate populations of cancer cells; and an exogenous nuclease, or a nucleic acid encoding said exogenous nuclease.

In some embodiments, said population of said plurality of cancer cells of each separate population comprises a genomic disruption in said single candidate gene. In some embodiments, at least 70%, 80%, or 90% of said plurality of cancer cells of each separate population comprises a genomic disruption in said single candidate gene. In some embodiments, each of said separate populations of cancer cells are contained with separate compartments of one or more arrays. In some embodiments, said plurality of separate populations of cancer cells comprises at least 10, 20, 30, 40, 50, 60, 70, 80, 90, 100, 200, 300, 400, 500, 600, 700, 800, 900, 1000, 5000, 10000, 50000, or 100000 separate populations of cancer cells.

In one aspect, provided herein are compositions comprising a plurality of separate cell populations that each comprise: i) a plurality of cancer cells that express an antigen; and ii) cells that express a cellular receptor, or functional fragment thereof, that binds to said antigen; wherein each of said plurality of cancer cells comprises an altered genome sequence of a single candidate gene, and wherein said single candidate gene is different for each of said separate cell populations.

In some embodiments, at least 70%, 80%, or 90% of said population of said plurality of cancer cells of each separate cell populations comprises said altered genome sequence of said single candidate gene.

In some embodiments, each of said separate cell populations are contained with separate compartments of one or more arrays.

In some embodiments, said plurality of separate cell populations comprises at least 10, 20, 30, 40, 50, 60, 70, 80, 90, 100, 200, 300, 400, 500, 600, 700, 800, 900, 1000, 5000, 10000, 50000, or 100000 separate cell populations.

In one aspect, provided herein are methods of screening a plurality of single candidate genes, said method comprising: a) obtaining a plurality of separate populations of cancer cells that express an antigen, wherein each population comprises a plurality of cancer cells; b) introducing into each of said separate populations of cancer cells a CRISPR system that comprises: i) a guide nucleic acid that binds a portion of a single candidate gene, wherein said single candidate gene is different for each of said separate populations of cancer cells; and ii) an exogenous nuclease, or a nucleic acid encoding said exogenous nuclease; thereby generating a plurality of separate populations of engineered cancer cells that comprise a genomic disruption in said single candidate gene, wherein said genomic disruption suppresses expression of said single candidate gene; c) performing an in vitro assay that comprises contacting in vitro said plurality of engineered cancer cells with a plurality of T cells that express a cellular receptor, or functional fragment thereof, that binds to said antigen; and d) obtaining a readout from said in vitro assay, to thereby determine an effect of said genomic disruption that suppresses expression of said single candidate gene on said plurality of separate populations of engineered cancer cells or said T cells that express a cellular receptor, or functional fragment thereof, that binds to said antigen.

In some embodiments, said readout comprises determining a level of cell death of each of said separate populations of engineered cancer cells. In some embodiments, said level of cell death is determined by flow cytometry or microscopy.

In some embodiments, said readout comprises determining a time to which a certain percentage of cells each of said separate populations of engineered cancer cells are killed. In some embodiments, said level of cell death is determined by flow cytometry or microscopy.

In some embodiments, said readout comprises determining a level of cytolytic activity of said plurality of T cells. In some embodiments, said level of cytolytic activity is determined by a chromium release assay, an electrical impedance assay, time-lapse microscopy, or a co-culture assay.

In some embodiments, said readout comprises determining a level of proliferation of said plurality of T cells. In some embodiments, said level of proliferation is determined by a Carboxyfluorescein Succinimidyl Ester (CFSE) assay, microscopy, an electrical impedance assay, or flow cytometry.

In some embodiments, said readout comprises determining a level of a factor expressed by said plurality of T cells. In some embodiments, said factor is a protein. In some embodiments, said protein is secreted from said population of engineered T cells. In some embodiments, said protein is a cytokine or chemokine. In some embodiments, said protein is a cell surface protein. In some embodiments, said expression is determined by flow cytometry, western blot, or ELISA.

In some embodiments, said antigen is an endogenous antigen. In some embodiments, said antigen is an exogenous antigen. The method of claim 111, wherein step a. comprises expressing said exogenous antigen in each of said separate populations of cancer cells.

In some embodiments, at least 60%, 65%, 70%, 75%, 80%, 85%, 90%, 95%, or 99% of cancer cells of each of said separate populations of cancer cells comprise said genomic disruption, in the absence of a selection step. In some embodiments, at least 80% of cancer cells of each of said separate populations of cancer cells comprise said genomic disruption, in the absence of a selection step. In some embodiments, at least 90% of cancer cells of each of said separate populations of cancer cells comprise said genomic disruption, in the absence of a selection step. In some embodiments, said percentage of cancer cells of each of said separate populations of cancer cells is determined by Tracking of Indels by Decomposition (TIDE) analysis.

In some embodiments, said cellular receptor is an immunomodulatory cellular receptor. In some embodiments, said cellular receptor is an exogenous cellular receptor. In some embodiments, said exogenous cellular receptor is integrated into the genome of said plurality of T cells.

In some embodiments, said exogenous cellular receptor is integrated into an endogenous gene sequence that encodes an exogenous cellular receptor. In some embodiments, said exogenous cellular receptor is integrated into a safe harbor site. In some embodiments, said safe harbor site is site is an AAVS site (e.g., AAVS1, AAVS2), CCR5, or hROSA26. In some embodiments, said exogenous cellular receptor is integrated into a portion of a gene that encodes a protein that functions as a negative regulator of an immune response of said plurality of T cells. In some embodiments, said integration decreases or inhibits expression of said protein that functions as a negative regulator of an immune response of said plurality of T cells. In some embodiments, said gene encodes for a protein selected from the group consisting of CISH, PD1, CTLA4, adenosine A2a receptor (ADORA), CD276, V-set domain containing T cell activation inhibitor 1 (VTCN1), B and T lymphocyte associated (BTLA), indoleamine 2,3-dioxygenase 1 (IDO1), killer cell immunoglobulin-like receptor, three domains, long cytoplasmic tail, 1 (KIR3DL1), lymphocyte-activation gene 3 (LAG3), hepatitis A virus cellular receptor 2 (HAVCR2), V-domain immunoglobulin suppressor of T-cell activation (VISTA), natural killer cell receptor 2B4 (CD244), hypoxanthine phosphoribosyltransferase 1 (HPRT), adeno-associated virus integration site 1 (AAVS1), or chemokine (C—C motif) receptor 5 (gene/pseudogene) (CCR5), CD160 molecule (CD160), T-cell immunoreceptor with Ig and ITIM domains (TIGIT), CD96 molecule (CD96), cytotoxic and regulatory T-cell molecule (CRTAM), leukocyte associated immunoglobulin like receptor 1 (LAIR1), sialic acid binding Ig like lectin 7 (SIGLEC7), sialic acid binding Ig like lectin 9 (SIGLEC9), tumor necrosis factor receptor superfamily member 10b (TNFRSF10B), tumor necrosis factor receptor superfamily member 10a (TNFRSF10A), caspase 8 (CASP8), caspase 10 (CASP10), caspase 3 (CASP3), caspase 6 (CASP6), caspase 7 (CASP7), Fas associated via death domain (FADD), Fas cell surface death receptor (FAS), transforming growth factor beta receptor II (TGFBRII), transforming growth factor beta receptor I (TGFBR1), SMAD family member 2 (SMAD2), SMAD family member 3 (SMAD3), SMAD family member 4 (SMAD4), SKI proto-oncogene (SKI), SKI-like proto-oncogene (SKIL), TGFB induced factor homeobox 1 (TGIF1), programmed cell death 1 (PD-1), cytotoxic T-lymphocyte-associated protein 4 (CTLA4), interleukin 10 receptor subunit alpha (IL10RA), interleukin 10 receptor subunit beta (IL10RB), heme oxygenase 2 (HMOX2), interleukin 6 receptor (IL6R), interleukin 6 signal transducer (IL6ST), c-src tyrosine kinase (CSK), phosphoprotein membrane anchor with glycosphingolipid microdomains 1 (PAG1), signaling threshold regulating transmembrane adaptor 1 (SIT1), forkhead box P3 (FOXP3), PR domain 1 (PRDM1), basic leucine zipper transcription factor, ATF-like (BATF), guanylate cyclase 1, soluble, alpha 2 (GUCY1A2), guanylate cyclase 1, soluble, alpha 3 (GUCY1A3), guanylate cyclase 1, soluble, beta 2 (GUCY1B2), prolyl hydroxylase domain (PHD1, PHD2, PHD3) family of proteins, or guanylate cyclase 1, soluble, beta 3 (GUCY1B3), egl-9 family hypoxia-inducible factor 1 (EGLN1), egl-9 family hypoxia-inducible factor 2 (EGLN2), egl-9 family hypoxia-inducible factor 3 (EGLN3), protein phosphatase 1 regulatory subunit 12C (PPP1R12C), NAD-dependent deacetylase sirtuin 2 (SIRT2), or Protein Tyrosine Phosphatase Non-Receptor Type 1 (PTPN1).

In some embodiments, at least 50%, 60%, 65%, 70%, 75%, 80%, 85%, 90%, 95%, or 99% of said plurality of T cells express said cellular receptor, in the absence of a selection step. In some embodiments, said percentage of T cells of said plurality of T cells is determined by flow cytometry or sequencing.

In some embodiments, said genomic disruption is a double strand break. In some embodiments, said nuclease is introduced using electroporation. In some embodiments, said nuclease is an endonuclease. In some embodiments, said endonuclease is selected from the group consisting of Cas1, Cas1B, Cas2, Cas3, Cas4, Cas5, Cas6, Cas7, Cas8, Cas9, Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx1S, Csf1, Csf2, CsO, Csf4, Cpf1, c2c1, c2c3, and Cas9HiFi. In some embodiments, said endonuclease is Cas9.

In some embodiments, said guide nucleic acid is a guide ribonucleic acid (gRNA).

In some embodiments, said guide nucleic acid comprises a phosphorothioate (PS) linkage, a 2′-fluoro (2′-F) modification, a 2′-O-methyl (2′-O-Me) linkage, a 2-O-Methyl 3phosphorothioate linkage, a S-constrained ethyl (cEt) modification, or any combination thereof. In some embodiments, said guide nucleic acid is introduced using electroporation.

In some embodiments, said cellular receptor is an exogenous cellular receptor introduced using electroporation. In some embodiments, said cellular receptor is an exogenous cellular receptor introduced using a viral vector.

In some embodiments, said viral vector is an adeno-associated virus (AAV) vector. In some embodiments, said AAV vector is selected from the group consisting of a recombinant AAV (rAAV) vector, a hybrid AAV vector, a chimeric AAV vector, a self-complementary AAV (scAAV) vector, a modified AAV vector, and any combination thereof. In some embodiments, said AAV vector is a chimeric AAV vector. In some embodiments, said chimeric AAV vector comprises a modification in at least one AAV capsid gene sequence.

In some embodiments, said cellular receptor is a T-cell receptor (TCR), B cell receptor (BCR), NK cell receptor, dendritic cell receptor, monocyte receptor, macrophage receptor, neutrophil receptor, eosinophil receptor, or a chimeric antigen receptor (CAR). In some embodiments, said cellular receptor is a T-cell receptor (TCR).

In some embodiments, said single gene is an immunomodulatory gene. In some embodiments, said single gene is a candidate immune checkpoint receptor ligand gene.

In some embodiments, said method further comprises cryopreserving said separate populations of engineered cancer cells.

In some embodiments, said method further comprises processing said readout to identify a candidate immunomodulatory gene.

In some embodiments, said processing comprises determining a criterion from at least one of: cytolytic activity, gene expression of said candidate immunomodulatory gene, intracellular location of a protein encoded by said candidate immunomodulatory gene, loss-of-function association with a human disease of said candidate immunomodulatory gene, a guide nucleic acid score of a guide nucleic acid that binds to a portion of said candidate immunomodulatory gene, existing drugs in development that target said candidate immunomodulatory gene, existing drugs that target said candidate immunomodulatory gene, or loss-of-function phenotype of said candidate immunomodulatory gene, or any combination thereof.

In some embodiments, said processing comprises determining a criterion from at least two, three, four, five, six, seven, or eight of: cytolytic activity, gene expression of said candidate immunomodulatory gene, intracellular location of a protein encoded by said candidate immunomodulatory gene, loss-of-function association with a human disease of said candidate immunomodulatory gene, a guide nucleic acid score of a guide nucleic acid that binds to a portion of said candidate immunomodulatory gene, existing drugs in development that target said candidate immunomodulatory gene, existing drugs that target said candidate immunomodulatory gene, or loss-of-function phenotype of said candidate immunomodulatory gene, or any combination thereof.

In some embodiments, said processing comprises ranking at least two candidate immunomodulatory genes according to said at least one criterion to produce ranked candidate immunomodulatory genes. In some embodiments, said processing comprises ranking at least 10, 100, 200, 300, 400, 500, 600, 700, 800, 900, 1000, 5000, 10000, 50000, or 100000 candidate immunomodulatory genes according to said at least one criterion.

In some embodiments, said processing comprises ranking at least two candidate immunomodulatory genes according to said at least two, three, four, five, six, seven, or eight criterion to produce ranked candidate immunomodulatory genes. In some embodiments, said processing comprises ranking at least 10, 100, 200, 300, 400, 500, 600, 700, 800, 900, 1000, 5000, 10000, 50000, or 100000 candidate immunomodulatory genes according to said at least one criterion.

In some embodiments, said processing comprises ranking at least 10, 100, 200, 300, 400, 500, 600, 700, 800, 900, 1000, 5000, 10000, 50000, or 100000 candidate immunomodulatory genes according to said at least two, three, four, five, six, seven, or eight criterion.

In some embodiments, said method further comprises selecting a top 10, 20, 30, 40, or 50 of said ranked candidate immunomodulatory genes to thereby generate a ranked output.

In some embodiments, said method further comprises identifying at least one of a gene family, a gene function, or an intracellular signaling pathway from said ranked output, to thereby generate an analyzed ranked output.

In some embodiments, said method further comprises correlating cytolytic activity of said analyzed ranked output, to thereby generate a cytolytic-correlated ranked output.

In some embodiments, said method further comprises ranking said candidate immunomodulatory genes from said cytolytic-correlated ranked output according to said intracellular location of a protein encoded by said candidate immunomodulatory gene.

In some embodiments, said method further comprises ranking said candidate immunomodulatory genes from said cytolytic-correlated ranked output according to said existing drug in development that targets said candidate immunomodulatory gene and said existing drug against said candidate immunomodulatory gene.

In some embodiments, said plurality of T cells comprises a plurality of CD8+ T cells. In some embodiments, said plurality of T cells comprises a plurality of CD4+ T cells. In some embodiments, said plurality of T cells comprises a plurality of CD4+ T cells and CD8+ T cells.

The method of any one of claims 95-163, wherein said plurality of T cells comprises a plurality of human cells. In some embodiments, said plurality of T cells comprises a plurality of primary cells. In some embodiments, said plurality of T cells comprises a plurality of ex vivo cells.

In some embodiments, said plurality of separate populations of cancer cells comprises at least 10, 20, 30, 40, 50, 60, 70, 80, 90, 100, 200, 300, 400, 500, 600, 700, 800, 900, 1000, 5000, 10000, 50000, or 100000 separate populations of cancer cells.

In some embodiments, said plurality of T cells comprises a transgene that encodes for a protein that improves immunomodulatory function of said T cells. In some embodiments, said transgene is integrated in the genome of said T cells. In some embodiments, said transgene is integrated into a safe harbor site. In some embodiments, said safe harbor site is site is an AAVS site (e.g., AAVS1, AAVS2), CCR5, or hROSA26. In some embodiments, said plurality of T cells comprises a genetic modification that enhances expression of a gene that encodes for a protein that improves immunomodulatory function of said T cells. In some embodiments, said transgene is integrated into a portion of a gene that encodes a protein that functions as a negative regulator of an immune response of said T cells.

In some embodiments, each of said separate populations of cancer cells comprise a genomic disruption in at least one gene. In some embodiments, said genomic disruption is mediated by a CRISPR system that comprises a gRNA that binds to a portion of said gene and a nuclease that mediates cleavage of genomic DNA. In some embodiments, said genomic disruption is a double strand break. In some embodiments, said at least one gene encodes a protein that that a negative regulator of an immune response. In some embodiments, said protein is a ligand of a checkpoint inhibitor. In some embodiments, said protein is a ligand of a checkpoint inhibitor selected from the group consisting of programmed cell death 1 (PD-1), cytotoxic T-lymphocyte-associated protein 4 (CTLA4), interleukin 10 receptor subunit alpha (IL10RA), interleukin 10 receptor subunit beta (IL10RB), adenosine A2a receptor (ADORA), CD276, V-set domain containing T cell activation inhibitor 1 (VTCN1), B and T lymphocyte associated (BTLA), indoleamine 2,3-dioxygenase 1 (IDO1), killer cell immunoglobulin-like receptor, three domains, long cytoplasmic tail, 1 (KIR3DL1), lymphocyte-activation gene 3 (LAG3), hepatitis A virus cellular receptor 2 (HAVCR2), V-domain immunoglobulin suppressor of T-cell activation (VISTA), natural killer cell receptor 2B4 (CD244), hypoxanthine phosphoribosyltransferase 1 (HPRT), adeno-associated virus integration site 1 (AAVS1), or chemokine (C—C motif) receptor 5 (gene/pseudogene) (CCR5), CD160 molecule (CD160), T-cell immunoreceptor with Ig and ITIM domains (TIGIT), CD96 molecule (CD96), cytotoxic and regulatory T-cell molecule (CRTAM), leukocyte associated immunoglobulin like receptor 1 (LAIR1), sialic acid binding Ig like lectin 7 (SIGLEC7), sialic acid binding Ig like lectin 9 (SIGLEC9), tumor necrosis factor receptor superfamily member 10b (TNFRSF10B), tumor necrosis factor receptor superfamily member 10a (TNFRSF10A), caspase 8 (CASP8), caspase 10 (CASP10), caspase 3 (CASP3), caspase 6 (CASP6), caspase 7 (CASP7), Fas associated via death domain (FADD), Fas cell surface death receptor (FAS), transforming growth factor beta receptor II (TGFBRII), transforming growth factor beta receptor I (TGFBR1), SMAD family member 2 (SMAD2), SMAD family member 3 (SMAD3), SMAD family member 4 (SMAD4), SKI proto-oncogene (SKI), SKI-like proto-oncogene (SKIL), TGFB induced factor homeobox 1 (TGIF1), heme oxygenase 2 (HMOX2), interleukin 6 receptor (IL6R), interleukin 6 signal transducer (IL6ST), c-src tyrosine kinase (CSK), phosphoprotein membrane anchor with glycosphingolipid microdomains 1 (PAG1), signaling threshold regulating transmembrane adaptor 1 (SIT1), forkhead box P3 (FOXP3), PR domain 1 (PRDM1), basic leucine zipper transcription factor, ATF-like (BATF), guanylate cyclase 1, soluble, alpha 2 (GUCY1A2), guanylate cyclase 1, soluble, alpha 3 (GUCY1A3), guanylate cyclase 1, soluble, beta 2 (GUCY1B2), prolyl hydroxylase domain (PHD1, PHD2, PHD3) family of proteins, or guanylate cyclase 1, soluble, beta 3 (GUCY1B3), egl-9 family hypoxia-inducible factor 1 (EGLN1), egl-9 family hypoxia-inducible factor 2 (EGLN2), egl-9 family hypoxia-inducible factor 3 (EGLN3), protein phosphatase 1 regulatory subunit 12C (PPP1R12C), NAD-dependent deacetylase sirtuin 2 (SIRT2), and Protein Tyrosine Phosphatase Non-Receptor Type 1 (PTPN1).

In some embodiments, said cancer cells express at least one exogenous protein. In some embodiments, said exogenous protein is a cell surface receptor. In some embodiments, said exogenous protein is an intracellular protein. In some embodiments, a transgene encoding said exogenous protein is integrated into the genome of said cancer cells. In some embodiments, said exogenous protein modulates the ability of an T cell to recognize and/or kill said cancer cells.

In some embodiments, each of said separate populations of T cells are contained with separate compartments of one or more arrays.

In one aspect, provided herein are compositions comprising a plurality of separate populations of cancer cells, wherein each separate population of cancer cells comprises a plurality of cancer cells that i) expresses an antigen; and ii); comprise a CRISPR system that comprises a guide nucleic acid that binds a portion of a single candidate gene, wherein said single candidate gene is different for each of said separate populations of cancer cells; and an exogenous nuclease, or a nucleic acid encoding said exogenous nuclease.

In some embodiments, said population of said plurality of cancer cells of each separate population comprises a genomic disruption in said single candidate gene. In some embodiments, at least 70%, 80%, or 90% of said plurality of cancer cells of each separate population comprises a genomic disruption in said single candidate gene. In some embodiments, each of said separate populations of cancer cells are contained with separate compartments of one or more arrays. In some embodiments, said plurality of separate populations of cancer cells comprises at least 10, 20, 30, 40, 50, 60, 70, 80, 90, 100, 200, 300, 400, 500, 600, 700, 800, 900, 1000, 5000, 10000, 50000, or 100000 separate populations of cancer cells.

In one aspect, provided herein are compositions comprising a plurality of separate cell populations that each comprise: i) a plurality of cancer cells that express an antigen; and ii) T cells that express a cellular receptor, or functional fragment thereof, that binds to said antigen; wherein each of said plurality of cancer cells comprises an altered genome sequence of a single candidate gene, and wherein said single candidate gene is different for each of said separate cell populations.

In some embodiments, at least 70%, 80%, or 90% of said population of said plurality of cancer cells of each separate cell populations comprises said altered genome sequence of said single candidate gene.

In some embodiments, each of said separate cell populations are contained with separate compartments of one or more arrays.

In some embodiments, said plurality of separate cell populations comprises at least 10, 20, 30, 40, 50, 60, 70, 80, 90, 100, 200, 300, 400, 500, 600, 700, 800, 900, 1000, 5000, 10000, 50000, or 100000 separate cell populations.

Provided herein are methods of screening a candidate gene comprising introducing into a cell i) a guiding polynucleic acid, or a nucleic acid encoding the guiding polynucleic acid, wherein the guiding polynucleic acid targets the candidate gene; and ii) an exogenous nuclease, or a nucleic acid encoding the exogenous nuclease; thereby generating an engineered cell comprising a genomic disruption in the candidate gene; b) contacting the engineered cell with an agent, thereby performing an in vitro assay; and c) determining a readout of the in vitro assay. In some embodiments, the readout comprises determining the level of cell proliferation. In some embodiments, the readout comprises determining the level of cell viability. In some embodiments, the readout comprises determining the level of cell death. In some embodiments, the level of proliferation can be determined by at least one of a Carboxyfluorescein Succinimidyl Ester (CFSE) assay, microscopy, an electrical impedance assay, or cytometry. In some embodiments, the level of cell viability and/or the level of cell death can be determined by microscopy, an electrical impedance assay, or cytometry. In some embodiments, the cells are immune cells, neuronal cells, liver cells, kidney cells, pancreatic cells, stomach cells, skin cells, heart cells, brain cells, muscle cells, lung cells, breast cells, small intestine cells, colon cells, anal cells, ovarian cells, cervical cells, or prostate cells. In some embodiments, the cells are cancer cells.

Provided herein is are methods of screening a candidate gene comprising a) expressing an exogenous cellular receptor, or a functional portion thereof, in an immune cell; introducing into the immune cell i) a guiding polynucleic acid, or a nucleic acid encoding the guiding polynucleic acid, wherein the guiding polynucleic acid targets the candidate gene; and ii) an exogenous nuclease, or a nucleic acid encoding the exogenous nuclease; thereby generating an engineered immune cell comprising a genomic disruption in the candidate gene; b) contacting the engineered immune cell with a cell expressing a cognate antigen of a T cell receptor or a functional portion thereof, thereby performing an in vitro assay; and c) determining a readout of the in vitro assay. In some cases, a method can further comprise selecting an immune cell that comprises the exogenous cellular receptor. In some cases, the readout comprises determining a level of cytolytic activity of the engineered immune cell. In some cases, the level of cytolytic activity can be determined by at least one of a co-culture assay, a chromium release assay, or time-lapse microscopy. In some cases, the readout comprises determining a level of proliferation of the engineered immune cell. In some cases, the level of proliferation can be determined by at least one of a Carboxyfluorescein Succinimidyl Ester (CFSE) assay, microscopy, an electrical impedance assay, or cytometry. In some cases, the readout comprises determining a level of a factor expressed by the engineered immune cell. In some cases, the factor can be selected from IL-2, IFNγ, TNFα, CD3, CD4, CD8, CD28, PD-1, CTLA4.

In some cases, the expression can be determined by flow cytometry, western blot, or ELISA. In some cases, a method can further comprise quantifying a level of the genomic disruption. In some cases, the quantifying comprises performing at least one of a Western blot analysis or a Tracking of Indels by Decomposition (TIDE) analysis. In some cases, the genomic disruption can be in an immune checkpoint gene. In some cases, a method can further comprise introducing a second genomic disruption to the engineered immune cell. In some cases, the second genomic disruption can be in an immune checkpoint gene. In some cases, the second genomic disruption can be in a gene that is not an immune checkpoint gene. In some cases, a method can further comprise cryopreserving the engineered immune cell. In some cases, the guiding polynucleic acid can be introduced non-virally. In some cases, the guiding polynucleic acid can be introduced virally. In some cases, the genomic disruption can be performed by an endonuclease. In an aspect, the endonuclease can be selected from the group consisting of: a Clustered Regularly Interspaced Short Palindromic Repeats (CRISPR) endonuclease, a Transcription activator-like effector nucleases (TALEN) endonuclease, an Argonaute endonuclease, and a Zinc Finger endonuclease. In some cases, an endonuclease can be a CRISPR endonuclease. In some cases, a CRISPR endonuclease can be Cas9.

In some embodiments, said genomic disruption in said candidate gene is introduced with an efficiency of at least 60%, 65%, 70%, 75%, 80%, 85%, 90%, 95%, or 99%. In some embodiments, said genomic disruption in said candidate gene is introduced with an efficiency of at least 80%. In some embodiments, said efficiency is measured by Tracking of Indels by Decomposition (TIDE) analysis. In some embodiments, said efficiency is measured by sequencing. In some embodiments, at least 60%, 65%, 70%, 75%, 80%, 85%, 90%, 95%, or 99% of said plurality of immune cells comprise said genomic disruption, in the absence of a selection step. In some embodiments, at least 80% of said plurality of immune cells comprise said genomic disruption, in the absence of a selection step. In some embodiments, said percentage of said plurality of immune cells if measured by Tracking of Indels by Decomposition (TIDE) analysis.

In some embodiments, said percentage of said plurality of immune cells if measured by sequencing. In some embodiments, said exogenous cellular receptor is introduced with an efficiency of at least 60%, 65%, 70%, 75%, 80%, 85%, 90%, 95%, or 99%. In some embodiments, said exogenous cellular receptor is introduced with an efficiency of at least 70%. In some embodiments, said efficiency is measured by flow cytometry. In some embodiments, said efficiency is measured by sequencing. In some embodiments, at least 60%, 65%, 70%, 75%, 80%, 85%, 90%, 95%, or 99% of the cells in said plurality express said exogenous cellular receptor, in the absence of a selection step. In some embodiments, at least 70% of the cells in said plurality express said exogenous cellular receptor, in the absence of a selection step. In some embodiments, said percentage of cells in said plurality is measured by flow cytometry. In some embodiments, said percentage of cells in said plurality is measured by sequencing.

In some cases, a) comprises contacting the immune cell with a viral particle comprising a nucleic acid encoding the exogenous cellular receptor or functional portion thereof. In some cases, a viral particle is an adeno-associated virus (AAV) particle. In some cases, a viral particle can be a modified adeno-associated virus (AAV) particle. In some cases, an exogenous cellular receptor can be a T-cell receptor (TCR) or a portion thereof or a chimeric antigen receptor (CAR) or a portion thereof. In some cases, an exogenous cellular receptor can be a TCR.

In some cases, a method can further comprise processing the readout to identify a candidate immunomodulatory gene or portion thereof. In some cases, the processing comprises determining a criterion from at least one of: cytolytic activity, gene expression of the candidate immunomodulatory gene or portion thereof, intracellular location of a protein generated by the candidate immunomodulatory gene or portion thereof, loss-of-function association with a human disease of the candidate immunomodulatory gene or portion thereof, a gRNA score of a gRNA that targets the candidate immunomodulatory gene or portion thereof, existing drug in development that targets the candidate immunomodulatory gene or portion thereof, existing drug against the candidate immunomodulatory gene or portion thereof, loss-of-function phenotype of the candidate immunomodulatory gene or portion thereof. In some cases, the processing comprises ranking candidate immunomodulatory genes or portions thereof according to the at least one criterion. In some cases, a method can further comprise selecting a top 10 of the ranked candidate immunomodulatory genes or portions thereof thereby generating a ranked output. In some cases, a method can further comprise identifying at least one of a gene family, a gene function, an intracellular signaling pathway from the ranked output, thereby generating an analyzed ranked output. In some cases, a method can further comprise correlating cytolytic activity of the analyzed ranked output thereby generating a cytolytic-correlated ranked output. In some cases, a method can further comprise ranking the candidate immunomodulatory genes or portions thereof from the cytolytic-correlated ranked output according to the intracellular location of a protein generated by the candidate immunomodulatory gene or portion thereof. In some cases, a score of the intracellular location of a protein can be low.

In some cases, a method can further comprise ranking the candidate immunomodulatory genes or portions thereof from the cytolytic-correlated ranked output according to the existing drug in development that targets the candidate immunomodulatory gene or portion thereof and the existing drug against the candidate immunomodulatory gene or portion thereof. In some cases, a score of the intracellular location of a protein can be low. In some cases, a method can further comprise repeating the method wherein the introducing a guiding polynucleic acid comprises a guiding polynucleic acid that targets the candidate immunomodulatory gene or portion thereof identified by the processing. In some cases, the immune cell can be a T cell, tumor infiltrating lymphocyte (TIL), or NK cell. In some cases, the T cell can be a CD8 cell. In some cases, the T cell can be a CD4 cell. In some cases, the cognate antigen binds the exogenous cellular receptor. In some cases, the guiding polynucleic acid can be modified. In some cases, the immune cell can be human.

Provided herein is a method of screening a candidate gene comprising: a) expressing an exogenous T-cell receptor (TCR) or a functional portion thereof, in an immune cell; b) introducing a genomic disruption in the candidate gene using a Clustered Regularly Interspaced Short Palindromic Repeats (CRISPR) system in the immune cell, thereby generating an engineered immune cell; c) contacting the engineered immune cell with a cell expressing a cognate antigen of a TCR or a functional portion thereof, thereby performing an in vitro assay; d) determining a readout of the in vitro assay; and e) processing the readout to identify a candidate immunomodulatory gene or portion thereof. In some cases, a method can further comprise selecting an immune cell that comprises the exogenous TCR or functional portion thereof. In some cases, a method can further comprise quantifying a level of the genomic disruption. In some cases, the quantifying comprises performing at least one of a Western blot analysis or a Tracking of Indels by Decomposition (TIDE) analysis. In some cases, the genomic disruption is in an immune checkpoint gene. In some cases, a method can further comprise introducing a second genomic disruption to the engineered immune cell. In some cases, a second genomic disruption can be in an immune checkpoint gene. In some cases, a second genomic disruption can be in a gene that is not an immune checkpoint gene.

In some embodiments, said genomic disruption in said candidate gene is introduced with an efficiency of at least 60%, 65%, 70%, 75%, 80%, 85%, 90%, 95%, or 99%. In some embodiments, said genomic disruption in said candidate gene is introduced with an efficiency of at least 80%. In some embodiments, said efficiency is measured by Tracking of Indels by Decomposition (TIDE) analysis. In some embodiments, said efficiency is measured by sequencing. In some embodiments, at least 60%, 65%, 70%, 75%, 80%, 85%, 90%, 95%, or 99% of said plurality of immune cells comprise said genomic disruption, in the absence of a selection step. In some embodiments, at least 80% of said plurality of immune cells comprise said genomic disruption, in the absence of a selection step. In some embodiments, said percentage of said plurality of immune cells if measured by Tracking of Indels by Decomposition (TIDE) analysis. In some embodiments, said percentage of said plurality of immune cells if measured by sequencing. In some embodiments, said exogenous TCR is introduced with an efficiency of at least 60%, 65%, 70%, 75%, 80%, 85%, 90%, 95%, or 99%. In some embodiments, said exogenous TCR is introduced with an efficiency of at least 70%. In some embodiments, said efficiency is measured by flow cytometry. In some embodiments, said efficiency is measured by sequencing. In some embodiments, at least 60%, 65%, 70%, 75%, 80%, 85%, 90%, 95%, or 99% of the cells in said plurality express said exogenous TCR, in the absence of a selection step. In some embodiments, at least 70% of the cells in said plurality express said exogenous TCR, in the absence of a selection step. In some embodiments, said percentage of cells in said plurality is measured by flow cytometry. In some embodiments, said percentage of cells in said plurality is measured by sequencing.

In some cases, a method can further comprise cryopreserving the engineered immune cell. In some cases, a CRISPR system is introduced non-virally. In some cases, a CRISPR system can be introduced virally. In some cases, a CRISPR system comprises a Cas9 endonuclease. In some cases, a) comprises contacting the immune cell with a viral particle comprising a nucleic acid encoding the exogenous cellular receptor or functional portion thereof. In some cases, the viral particle can be an adeno-associated virus (AAV) particle. In some cases, the viral particle can be a modified adeno-associated virus (AAV) particle. In some cases, d) comprises determining a cytolytic activity of the engineered immune cell.

In some cases, cytolytic activity is determined by at least one of a co-culture assay, a chromium release assay, or time-lapse microscopy. In some cases, d) comprises determining proliferation of the engineered immune cell. In some cases, proliferation can be determined by at least one of a Carboxyfluorescein Succinimidyl Ester (CFSE) assay, microscopy, or cytometry. In some cases, d) comprises determining a factor expression of the engineered immune cell. In some cases, the factor can be selected from IL-2, IFNγ, TNF, CD3, CD4, CD8, CD28, PD-1, CTLA4. In some cases, expression can be determined by flow cytometry, western blot, or ELISA.

In some cases, processing comprises determining a criterion from at least one of: cytolytic activity, gene expression of the candidate immunomodulatory gene or portion thereof, intracellular location of a protein generated by the candidate immunomodulatory gene or portion thereof, loss-of-function association with a human disease of the candidate immunomodulatory gene or portion thereof, a gRNA score of a gRNA that targets the candidate immunomodulatory gene or portion thereof, existing drug in development that targets the candidate immunomodulatory gene or portion thereof, existing drug against the candidate immunomodulatory gene or portion thereof, loss-of-function phenotype of the candidate immunomodulatory gene or portion thereof. In some cases, processing comprises ranking candidate immunomodulatory genes or portions thereof according to the at least one criterion. In some cases, a method can further comprise selecting a top 10 of the ranked candidate immunomodulatory genes or portions thereof thereby generating a ranked output. In some cases, a method can further comprise identifying at least one of a gene family, a gene function, an intracellular signaling pathway from the ranked output, thereby generating an analyzed ranked output. In some cases, a method can further comprise correlating cytolytic activity of the analyzed ranked output thereby generating a cytolytic-correlated ranked output. In some cases, a method can further comprise ranking the candidate immunomodulatory genes or portions thereof from the cytolytic-correlated ranked output according to the intracellular location of a protein generated by the candidate immunomodulatory gene or portion thereof. In some cases, a score of the intracellular location of a protein can be low. In some cases, a method can further comprising ranking the candidate immunomodulatory genes or portions thereof from the cytolytic-correlated ranked output according to the existing drug in development that targets the candidate immunomodulatory gene or portion thereof and the existing drug against the candidate immunomodulatory gene or portion thereof. In some cases, a score of the intracellular location of a protein can be low. In some cases, a method can further comprise repeating the method wherein the introducing a guiding polynucleic acid comprises a guiding polynucleic acid that targets the candidate cancer therapeutic gene or portion thereof identified by the processing.

In some cases, an immune cell can be a T cell, tumor infiltrating lymphocyte (TIL), or NK cell. In some cases, the T cell can be a CD8 cell. In some cases, a T cell can be a CD4 cell. In some cases, a cell expressing a cognate antigen of a T cell receptor or a functional portion thereof binds the TCR. In some cases, a CRISPR system can be modified. In some cases, an immune cell can be human. In some cases, a cell expressing a cognate antigen of a T cell receptor or a functional portion thereof can be a cancer cell.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 provides an exemplary scheme for the generation of populations of primary human T cells expressing an exogenous TCR of known specificity.

FIG. 2 provides an exemplary scheme for evaluating the effect of candidate immunomodulatory gene disruption in primary human T cells.

FIG. 3 illustrates an exemplary use of algorithms to aid the ranking, selection, or identification of candidate immunomodulatory genes.

FIG. 4 illustrates the cyclical or iterative implementation of various components of the disclosure for the identification of immunomodulatory genes.

FIG. 5 provides illustrative outlines of algorithm workflows. FIG. 5A provides an illustrative outline of an algorithm to rank candidate immunomodulatory genes based on screening assays and other weighted parameters. FIG. 5B provides an illustrative outline of an algorithm for iterative selection of candidate immunomodulatory genes to screen. FIG. 5C provides an illustrative outline of an algorithm for identification of druggable immunomodulatory genes related to candidate genes that are poor drug targets.

FIG. 6 is a bar graph showing the efficiency of checkpoint gene knockout using a CRISPR system described herein comprising a gRNA directed four different checkpoint genes (targets). Cryopreserved CD3+T were thawed, stimulated with CD3 and CD28 Dynabeads for 3 days, and cultured in ex-vivo growth media with 10% human serum, IL2, IL7, and IL15. The Dynabeads were removed and the cells returned to fresh growth media for 2 hours prior to transfection. Transfection was conducted using the Neon transfection system and 3×10\hat{ }5 T cells were transfected in a 10 μl neon tip with 1.5 μg of Cas9 mRNA and 0.5 μg of gRNA. The T cells were placed into fresh growth media at a density of 1×10\hat{ }6 cells/ml. Samples were taken for Tide analysis to check editing efficiencies at 3 days post transfection.

FIG. 7 is a plot from a FACS analysis showing the efficiency of TCR integration using AAV vector described herein. Three generations of the vector are shown, with the third generation having 78% knock in efficiency. Cryopreserved CD3+ T were thawed, stimulated with CD3 and CD28 Dynabeads for 3 days, and cultured in ex-vivo growth media with 10% human serum, IL2, IL7, and IL15. The Dynabeads were removed and the cells returned to fresh growth media for 2 hours prior to transfection. Transfection was conducted using the Neon transfection system and 3×10\hat{ }5 T cells were transfected in a 10 μl neon tip with 1.5 μg of Cas9 mRNA and 0.5 μg of gRNA. The T cells were placed into fresh growth media at a density of 1×10\hat{ }6 cells/ml. Two hours post transfection an AAV donor virus comprising the exogenous TCR construct was added to the medium (at a MOI of 1×10\hat{ }6). Samples were taken for Tide analysis to check editing efficiencies at 3 days post transfection. The differences between the generations of this protocol that lead to improved percentages of TCR knock-in relate to the other work developed for a clinical cell therapy protocol to CRISPR edit T cells.

FIG. 8A provides an illustration of a splice acceptor KRAS-G12D specific TCR transgene construct and its CRISPR/AAV mediated insertion at the endogenous TRAC locus. FIG. 8B provides an illustration of the interaction between an engineered T cell expressing e.g., a KRAS G12D specific TCR and a COS7 MHCI+ cell pulsed with G12D peptide.

FIG. 9 provides an illustration of an embodiment of methods of generating modified cells (e.g., T cells) descried herein, wherein CD8+ T cells are isolated from a subject (e.g., a human subject), engineered to knock in a TCR specific for a selected antigen and knockout a gene of interest (e.g., an immune checkpoint gene, e.g., CISH, PD1, CTLA4) with high efficiency, optionally enriching for the genomically edited cells, optionally confirming DNA disruption and protein loss by an assay, e.g., western blot, TiDE, and using the cells in a screening method described herein, or optionally cryopreserving the cells for later use in an assay described herein.

FIG. 10 shows an illustration of an embodiment of methods of identifying immune checkpoint proteins in modified cells described herein, wherein a pure population of edited and optionally enriched cells (e.g., T cells as described in FIG. 10) are arrayed (e.g., into a 96 well plate) such that each well comprises T cells expressing a transgenic TCR and a single immune checkpoint protein knockout. Subsequently, target cells such as cells presenting peptide antigen recognized by the transgenic TCR are added to the array. T cell mediated cytolysis of the target cells is read to identify T cells with enhanced cytotoxicity when specific genes are knocked out. This method can also be used to show synergistic or additive effects when combinations of genes are knocked out.

FIG. 11 shows an illustration of an embodiment of methods of identifying immune checkpoint proteins in modified cells described herein.

FIG. 12 is a plot from a FACS analysis showing enrichment of CRISPR edited TCR knock in T cells within less than a 72-hour time frame, thereby increasing the effectiveness and range of antigen-specific cytolytic killing in the assay. The a 24 well plate was coated with anti-TCR antibody (4 μg/mL in PBS). 250 μL of medium per well of the 24 well plate was added and left overnight at 4° C. The supernatant was removed before adding 5×10\hat{ }5 TCR knock in T cells. The T cells were added 7 days post TCR knock in editing. 2 μg of an anti-CD28 monoclonal antibody was added to the T cells, and the T cells cultured at 37° C. in 5% CO₂ for 7 days, feeding and replacing media as needed.

FIG. 13 shows a plot from a T cell cytolytic assay using the xCelligence platform. The plot shows the kinetics of T cell killing up to 120 hours post combination of the T cells and target cells. The assay shows a robust window of activity, both in magnitude of killing and in kinetics of the response, in which gene targets that increase the cancer antigen specific killing can be identified. The unhindered proliferation of the COS-7 target cells was used as a base line level of death, set to zero. The true antigen specific killing is the different in cell death between G12D TCR engineered CD8+ T cells responding to COS-7 cells expressing the G12 WT peptide. The assay window to identify genes that increase cancer antigen specific killing is the difference in cell death between the maximum control (all cells killed by Triton X addition) and the response of antigen-specific TCR engineered CD8+ T cells cocultured with COS-7 cells expressing the cognate peptide antigen (KRAS G12D).

FIG. 14 shows a plot from a T cell cytolytic assay using the xCelligence platform. The plot shows both an acute killing phase within the first 24 hours and a later serial killing phase. The CISH knockout CD8+ T cells showed an elevated level of cancer antigen specific killing compared to the WT CD8+ T cells. Also, loss of CISH increases the rapidity of cancer cell killing seen at the earlier timepoints (e.g., acute killing phase) as well as increasing the overall magnitude of antigen-specific cell killing.

FIG. 15A is a bar graph showing percent cytolysis of CISH knockout CD8+ T cells and WT CD8+ T cells. CISH deficient T cells showed enhanced cytotoxicity in response to mutation specific tumor antigens at 16 hours, 72 hours, and 96 hours post combination of the T cells and target cells compared to WT. Cytolysis was measured using the xCelligence platform. FIG. 15B shows cytolysis of CISH knockout CD8+ T cells and WT CD8+ T cells 16 hours after combination of the T cells and target cells using CellTox dye-based assay. The data produced using the xCelligence platform as presented in FIG. 16A correlates with that produced using the CellTox dye-based assay.

FIG. 16 is a bar graph showing the results of a screening assay measuring the fold increase in specific cell lysis 16 hours after the combination of the CD8+ T cells and target cells. Two positive hits were identified that gave a 1.8-fold and a 1.6-fold increase in killing over wildtype T cells.

FIG. 17 is a plot showing from a T cell cytolytic assay using the xCelligence platform, screening 11 different target genes. The screen shows robust antigen specific killing response with the CD8+ T cells expressing the engineered TCR.

FIG. 18A is a plot showing from a T cell cytolytic assay using the xCelligence platform, screening 10 different target genes. The screen identified one target gene, wherein the knockout of the gene in the TCR transgenic CD8+ T cells gave a rapid increase in T cell killing compared to the WT T cells. FIG. 18B is a bar showing the results of the screening assay in FIG. 19A at 16 hours after the combination of the CD8+ T cells and target cells.

FIG. 19 shows an illustration of an embodiment described herein, wherein the cytotoxicity assay is combined with software to identify new druggable target genes. The software searches numerous biological databases and search strategies to select genes for CRISPR mediated knockout in subsequent experimental rounds. The software uses several algorithms that compete against each other and the more successful algorithms are more frequently used for subsequent screens. The final result is a list of enriched hits, identified genes that lead to higher selected functional output (e.g., magnitude T cell killing). The searches can include, for example biological process, cellular component, molecular function, nearest neighbor, and Steiner trees.

FIG. 20 shows an illustration of the algorithm input-output. Data generated from CRISPR T cell cytolytic screen is input and a network based on statistics is used to find additional targets (e.g., Go Term ontology searching based on molecular function). The targets for the next round of screening are output, and the software can perform an additional analysis of the gene targets to add weighting based on parameters of drugability (e.g., expression level in a target cell type (e.g., T cells), trackability, cellular localization, existing drugs on the market targeting the gene, whether drugs targeting the gene are in clinical trials, and whether the gene is associated with a human disease. Based on the parameters the software outputs a refined list of gene targets for the next round of screening.

FIG. 21 shows an illustration of the method incorporates iterative machine learning to evolve and improve the software and provide faster identification of high value drug targets and combination gene effects. Iterative improvement in the ability of the software to predict genes important for T cell cytolytic activity enables more rapid identification of highly druggable targets. Each round of CRISPR screening generates data to improve the software and select dominant decision-making algorithms. The software also identifies genes that are predicted to lead to significant improvements in T cell killing when perturbed in combination.

### DETAILED DESCRIPTION

The following description and examples illustrate embodiments of the disclosure in detail. It is to be understood that this disclosure is not limited to the particular embodiments described herein and as such can vary. Those of skill in the art will recognize that there are numerous variations and modifications of this invention, which are encompassed within its scope.

All publications, patents, and patent applications herein are incorporated by reference to the same extent as if each individual publication, patent, or patent application was specifically and individually indicated to be incorporated by reference. In the event of a conflict between a term herein and a term in an incorporated reference, the term herein controls.

### INTRODUCTION

The identification of new genes of interest for disruption in a specific cell population may lead to new treatments associated with diseases or disorders. For example, the identification of new immunomodulatory genes may lead to new treatments for cancer or other disorders associated with those genes, e.g., autoimmune diseases. The present disclosure provides, inter alia, methods of identifying immunomodulatory genes, including, for example, methods that allow for large scale screens of primary human T cells. Prior screens for immunomodulatory genes relied on readouts of limited relevance to cancer cell recognition and killing (e.g., T-cell proliferation), as well as relied on cell lines rather than primary cells, or involved pooling candidate immunomodulatory gene disruptions in one experimental condition.

The methods provided herein allow, for example, arrayed screening of primary human T cells for cytolytic killing of cancer cells in an antigen-dependent manner. Without wishing to be bound by theory, assaying for cytolytic activity can provide benefits over assaying for T cell proliferation because cytolytic activity can be a more relevant metric with respect to a candidate gene's ability to confer cancer cell-killing activity to an immune cell such as a T cell (e.g., when the candidate gene is knocked out, especially in an immune cell expressing a tumor-reactive TCR) is especially true when assayed in primary cells, as these conditions more accurately reflect physiological processes than cell lines. Moreover, these more-accurate methods interact synergistically with the machine learning systems described herein because they provide the machine learning system with better data with which to make recommendations for candidate immunomodulatory genes (i.e., better data in yields better predictions out). Additionally, proliferation of T cells ex vivo in the absence of the vast milieu of cytokines, chemokines and other in vivo factors is artificial and likely a poorer predictor of target and drug responses in human patients than a test for cytolysis. A cytolytic assay reproduces the cognate TCR and antigen interaction seen in vivo in the tumor microenvironment and enables formation of the immune synapse and mobilization of effector functions that lead to direct cancer cell killing, which is closer to the conditions important in cancer immunotherapy. The methods of the present disclosure are also powerful in that when an arrayed cytolytic assay (e.g., using CRISPR for candidate gene disruption) is combined with high efficiency T cell engineering, T cells can be specifically modified to knock out gene targets before being subjected to the CRISPR libraries for screening. This could include immune checkpoint knockouts (e.g., in PD-1, CTLA-4, and/or CISH) in T cells being screened to discover genes that synergistically or additively lead to better cancer killing. In iterative rounds of screening, this could also include novel targets that are identified within the screen itself. The algorithms described herein could also be programmed to predict likely targets that are synergistic when knocked out together, such as by considering genes that individually show a positive cytolytic response and adding knowledge about their gene pathway to consider redundancies and infer relationships that lead to rational choices of genes to screen in combination.

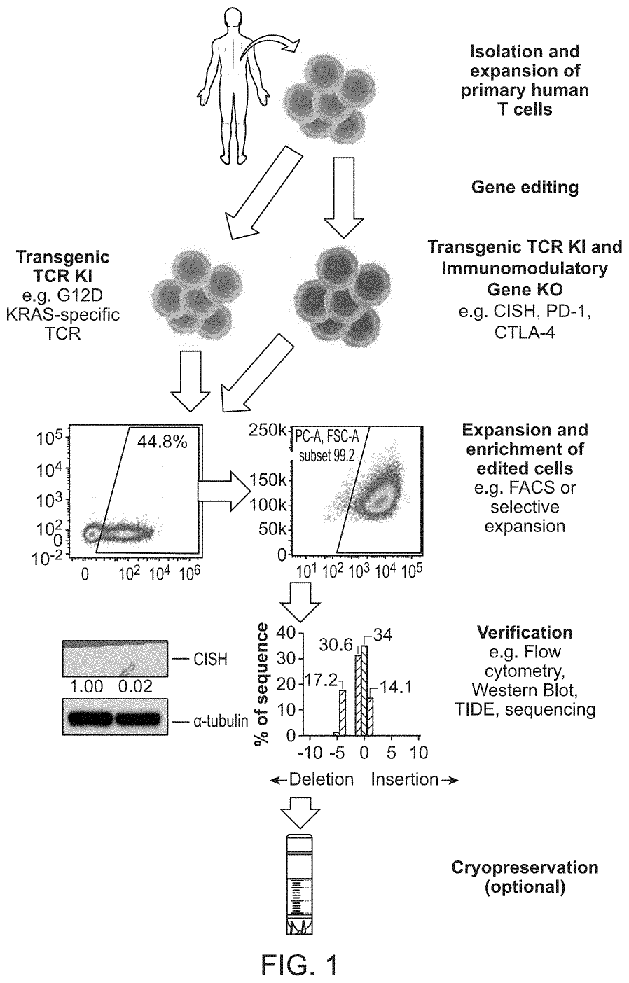

In some embodiments, populations of primary human T cells expressing an exogenous TCR of known specificity are generated as outlined in FIG. 1. In some embodiments, primary human T cells are isolated, expanded, and an exogenous T cell receptor (TCR) of known specificity expressed in the T cells. In some embodiments, gene disruptions are introduced into the T cells at this stage (e.g., disruptions of an endogenous TCR, an immune checkpoint gene, or a combination thereof). In some embodiments, T cells expressing the TCR of known specificity are then enriched (e.g., via fluorescent activated cell sorting (FACS)) and expanded. In some embodiments, the resulting T cells are tested for gene disruption and/or expression of exogenous TCR (e.g., via flow cytometry, Western Blot, tracking of indels by decomposition (TIDE), or sequencing). In some embodiments, the T cells are cryopreserved for later use.

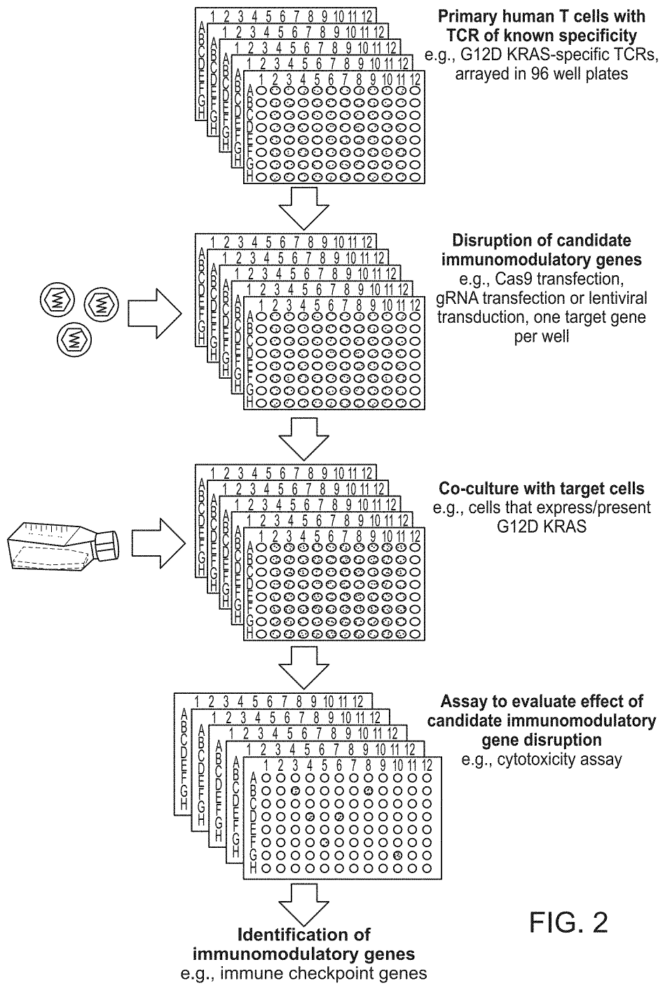

In some embodiments, candidate immunomodulatory genes are disrupted and the effects of gene disruption are evaluated as outlined in FIG. 2. For example, in some embodiments, disruption of candidate immunomodulatory genes is carried out in primary human T cells expressing an exogenous TCR of known specificity. In some embodiments, disruption of candidate immunomodulatory genes is carried out in an arrayed format, and involves transfection of a nuclease (e.g., Cas9) and transduction of guide RNAs (gRNAs). This can result in arrayed populations of primary human T cells, all of which express a TCR of known specificity, but featuring disruption of different candidate immunomodulatory genes in different experimental conditions (e.g., one gene disrupted in each well of a 96-well plate). In some embodiments, the arrayed T cells are then co-cultured with target cells that express or present a cognate antigen for the TCR of known specificity or a functional portion thereof (e.g., primary cells, primary cancer cells, or a cell line). In some embodiments, the effect of candidate immunomodulatory gene disruption on T cell response to the target cells is then be evaluated (e.g., in some embodiments, T cell killing of target cells via a cytotoxicity assay; in some embodiments cytokine production, proliferation, activation, or memory differentiation). In some embodiments, the T cells comprise a disruption of one or more candidate immunomodulatory genes and/or one more known immunomodulatory genes. Disruption of two or more genes can be beneficial, as it facilitates screening for synergistic or additive effects. In some embodiments, the co-culture is conducted in the presence of immunosuppressive agents (e.g., an adenosine receptor agonist or TGF-β) in order to screen for disruptions that overcome immune suppression.

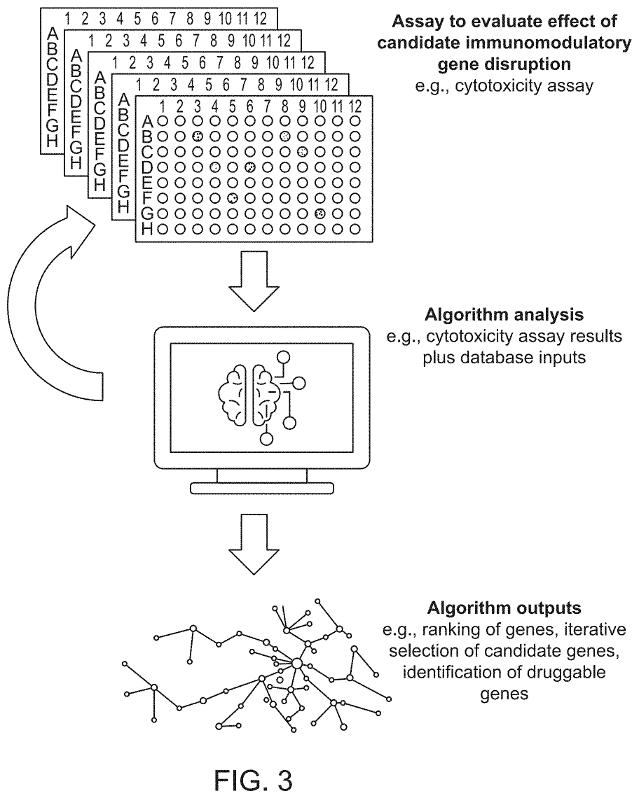

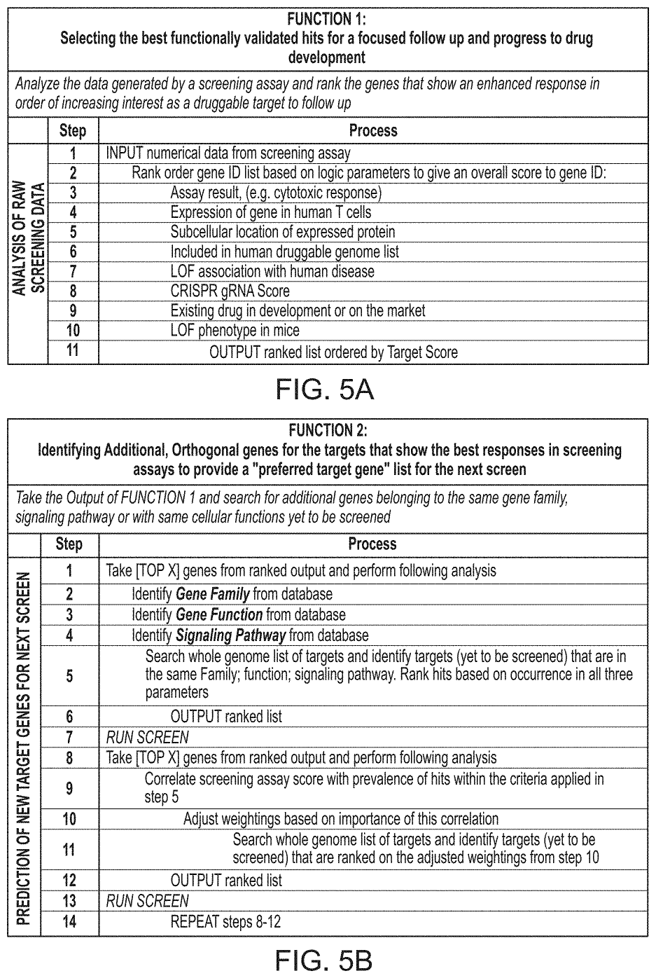

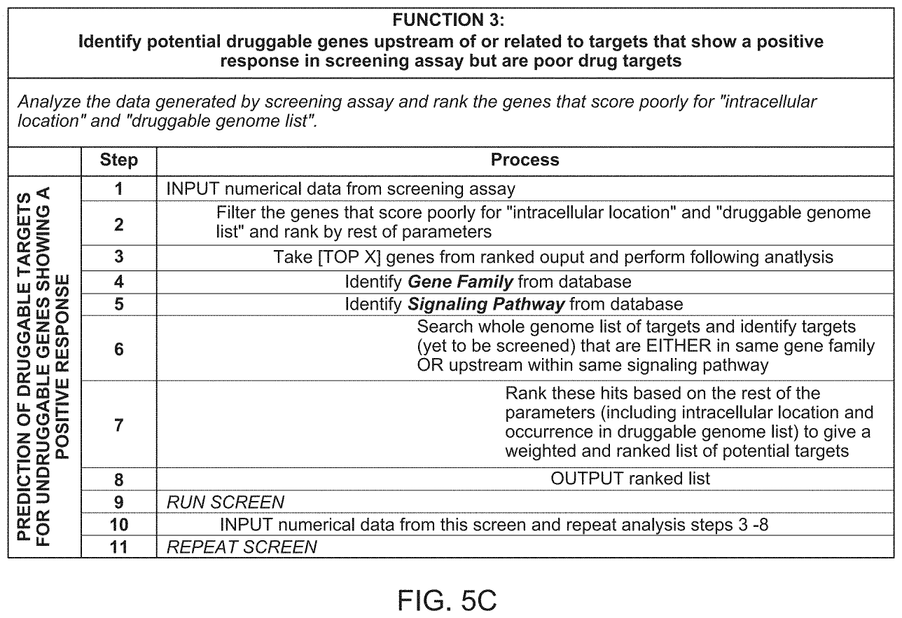

In some embodiments, algorithms are used to aid the prediction, ranking, selection, or identification of candidate immunomodulatory genes, as illustrated by FIG. 3. For example, in some embodiments, the results of an assay testing the effect of candidate immunomodulatory gene disruptions are input into an algorithm, which can combine that data with other data, for example, prior assay results or database entries, and provide an output of ranked genes for follow-up experiments. In some embodiments, algorithms are used to rank candidate immunomodulatory genes based on screening assays and other weighted parameters, as illustrated by example 24 and FIG. 5A. In some embodiments, algorithms are used for iterative selection of candidate immunomodulatory genes to screen, as illustrated by example 25 and FIG. 5B. In some embodiments, algorithms are used to identify druggable immunomodulatory genes related to candidate genes that are poor drug targets, as illustrated by example 26 and FIG. 5C.

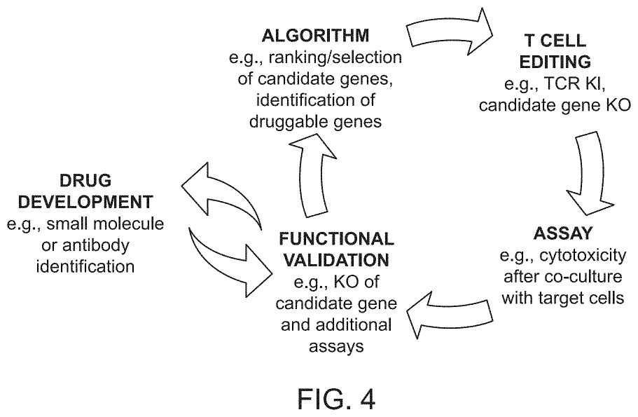

In some embodiments, the various components outlined above are executed in a cyclical or iterative fashion as illustrated by FIG. 4. For example, in some embodiments, a screening assay is run wherein multiple candidate immunomodulatory genes are tested. In some embodiments, the results are input into an algorithm, which outputs a ranked list of candidate genes to screen in a subsequent assay. The assay can be run, results input into an algorithm, and the cycle can repeat.

In some embodiments, the methods provided herein identify immunomodulatory genes, which can be targeted in drug development, for example, development of small molecules, biologics, or cell therapies to treat cancer or other disorders associated with immunomodulatory genes. In some embodiments, the methods provided herein can identify immune checkpoint genes.

### Immunomodulatory Genes and Immune Checkpoint Genes

Disclosed herein are methods for identifying immunomodulatory genes. Immunomodulatory genes can affect the progression of a range of diseases including cancer. Thus, identified immunomodulatory genes (for example, identified using the methods of the present disclosure) may be targets for the treatment of cancer or other diseases involving those genes.

Immune responses directed against cancer cells are important in limiting the growth or spread of cancer. For example, T cells can recognize mutated self-antigens (neoantigens) via T cell receptors (TCRs), which can lead to an immune response directed against the mutated cell, for example, killing of the mutated cell by cytotoxic CD8 T cells, or the production of inflammatory cytokines. Some cancerous cells, however, can negatively regulate immune responses, which can contribute to cancer cell survival and spread. An immune response can be down-regulated through mechanisms involving immunomodulatory genes.

Immunomodulatory genes contribute to inhibiting, down-regulating, or limiting an immune response. An immunomodulatory gene can be part of a feedback loop that regulates the amplitude of an immune response. An immunomodulatory gene can, for example, inhibit immune cell expansion, inhibit immune cell functional avidity, inhibit cytokine production, inhibit cytokine polyfunctionality, inhibit cytolytic or cytotoxic killing of target cells, inhibit immune cell migration, inhibit immune cell degranulation, inhibit immune cell sensitivity to an activating stimulus, inhibit immune cell persistence, inhibit immune cell survival, promote immune cell apoptosis, promote immune cell anergy, promote immune cell exhaustion, or any combination thereof. Immunomodulatory genes can comprise coinhibitory receptors and ligands thereof, which can, for example, lead to signaling cascades that inhibit, down-regulate, or limit an immune response. In particular, the methods described herein can be used to identify immunomodulatory genes that, when disrupted, have cancer cell-killing (cytolytic) activity. By assaying directly for cytolytic activity, the methods provided herein can provide an advantage over methods that do not assay for this activity.

Immunomodulatory genes can comprise checkpoint genes. Therapies that target immune checkpoint genes can comprise checkpoint inhibitors. Some checkpoint inhibitors have demonstrated efficacy in the treatment of cancer, for example, anti-PD-L1 monoclonal antibodies. Thus, the identification of new immunomodulatory genes or checkpoint genes could provide targets for the development of new therapies for cancer.

Therapies targeting an immunomodulatory gene can lead to upregulation of an immune response, for example, an anti-cancer immune response. Therapies targeting an immunomodulatory gene can target an immunomodulatory gene, a protein product of an immunomodulatory gene, or a ligand, interaction partner, or activating factor of an immunomodulatory gene or protein product thereof.

Immune cells of the disclosure comprise, for example, T cells, CD4 T cells, CD8 T cells, alpha-beta T cells, gamma-delta T cells, T regulatory cells (Tregs), cytotoxic T lymphocytes, T_{H}1 cells, T_{H}2 cells, T_{H}17 cells, T_{H}9 cells, Natural killer T cells (NKTs), Natural killer cells (NKs), Innate Lymphoid Cells (ILCs), B cells, plasma cells, antigen presenting cells (APCs), monocytes, macrophages, dendritic cells, plasmacytoid dendritic cells, neutrophils, tumor-infiltrating lymphocytes (TILs), mast cells, or a combination thereof. In some embodiments, immune cells of the disclosure are patient-derived T cells or TILs that naturally express an endogenous TCR against a tumor antigen. These include, for example, isolated and expanded mutation-reactive TILs. In some embodiments, APCs are cell lines or autologous cells patient-derived cells expressing the cognate antigen.

### Expansion of Immune Cells

The present disclosure provides methods for screening primary immune cells (e.g., human T cells) for immunomodulatory genes. In some embodiments, to generate a sufficient number of primary immune cells (e.g., human T cells) for screening assays, the primary cells are expanded.

Generally, in some embodiments, the cells of the disclosure are expanded by contact with a surface having attached thereto an agent that can stimulate a CD3 TCR complex associated signal and a ligand that can stimulate a co-stimulatory molecule on the surface of the T cells. In particular, in some embodiments, T cell populations are stimulated in vitro such as by contact with an anti-CD3 antibody or antigen-binding fragment thereof, or an anti-CD2 antibody immobilized on a surface, or by contact with a protein kinase C activator (e.g., bryostatin) sometimes in conjunction with a calcium ionophore. In some embodiments, for co-stimulation of an accessory molecule on the surface of the T cells, a ligand that binds the accessory molecule is used. For example, a population of T cells can be contacted with an anti-CD3 antibody and an anti-CD28 antibody, under conditions that can stimulate proliferation of the T cells. In some cases, 4-1BB can be used to stimulate cells. For example, cells can be stimulated with 4-1BB and IL-21 or another cytokine.

To stimulate proliferation of either CD4 T cells or CD8 T cells, an anti-CD3 antibody and an anti-CD28 antibody can be used. For example, the agents providing a signal may be in solution or coupled to a surface. The ratio of particles to cells may depend on particle size relative to the target cell. In further embodiments, the cells, such as T cells, can be combined with agent-coated beads, where the beads and the cells can be subsequently separated, and optionally cultured. Each bead can be coated with either anti-CD3 antibody or an anti-CD28 antibody, or in some cases, a combination of the two. In an alternative embodiment, prior to culture, the agent-coated beads and cells are not separated but are cultured together. Cell surface proteins may be ligated by allowing paramagnetic beads to which anti-CD3 and anti-CD28 can be attached (3×28 beads) to contact the T cells. In one embodiment the cells and beads (for example, DYNABEADS® M-450 CD3/CD28 T paramagnetic beads at a ratio of 1:1) are combined in a buffer, for example, phosphate buffered saline (PBS) (e.g., without divalent cations such as, calcium and magnesium).

Any cell concentration may be used. For example, in some embodiments, the concentration of cells prior to expansion is about 1×10³, 2×10³, 3×10³, 4×10³, 5×10³, 6×10³, 7×10³, 8×10³, 9×10³, 1×10⁴, 2×10⁴, 3×10⁴, 4×10⁴, 5×10⁴, 6×10⁴, 7×10⁴, 8×10⁴, 9×10⁴, 1×10⁵, 2×10⁵, 3×10⁵, 4×10⁵, 5×10⁵, 6×10⁵, 7×10⁵, 8×10⁵, 9×10⁵, 1×10⁶, 2×10⁶, 3×10⁶, 4×10⁶, 5×10⁶, 6×10⁶, 7×10⁶, 8×10⁶, 9×10⁶, 1×10⁷, 2×10⁷, 3×10⁷, 4×10⁷, 5×10⁷, 6×10⁷, 7×10⁷, 8×10⁷, 9×10⁷, 1×10⁸, 2×10⁸, 3×10⁸, 4×10⁸, 5×10⁸, 6×10⁸, 7×10⁸, 8×10⁸, 9×10⁸, 1×10⁹, 2×10⁹, 3×10⁹, 4×10⁹, 5×10⁹, 6×10⁹, 7×10⁹, 8×10⁹, or 9×10⁹ cells per mL. In some embodiments, the concentration of cells prior to expansion is at least about 1×10³, 2×10³, 3×10³, 4×10³, 5×10³, 6×10³, 7×10³, 8×10³, 9×10³, 1×10⁴, 2×10⁴, 3×10⁴, 4×10⁴, 5×10⁴, 6×10⁴, 7×10⁴, 8×10⁴, 9×10⁴, 1×10⁵, 2×10⁵, 3×10⁵, 4×10⁵, 5×10⁵, 6×10⁵, 7×10⁵, 8×10⁵, 9×10⁵, 1×10⁶, 2×10⁶, 3×10⁶, 4×10⁶, 5×10⁶, 6×10⁶, 7×10⁶, 8×10⁶, 9×10⁶, 1×10⁷, 2×10⁷, 3×10⁷, 4×10⁷, 5×10⁷, 6×10⁷, 7×10⁷, 8×10⁷, 9×10⁷, 1×10⁸, 2×10⁸, 3×10⁸, 4×10⁸, 5×10⁸, 6×10⁸, 7×10⁸, 8×10⁸, 9×10⁸, 1×10⁹, 2×10⁹, 3×10⁹, 4×10⁹, 5×10⁹, 6×10⁹, 7×10⁹, 8×10⁹, or 9×10⁹ cells per mL.

In some embodiments, the mixture is cultured or expanded for about several hours (e.g., about 3 hours) to about 21 days or any hourly integer value in between. In some embodiments, cells are cultured or expanded, for example, for about 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 102, 108, 114, 120, 126, 132, 138, 144, 150, 156, 162, 168, 174, 180, 186, 192, 198, 204, 210, 216, 222, 228, 234, 240, 246, 252, 258, 264, 270, 276, 282, 288, 294, 300, 306, 312, 318, 324, 330, 336, 342, 348, 354, 360, 366, 372, 378, 384, 390, 396, 402, 408, 414, 420, 426, 432, 438, 444, 450, 456, 462, 468, 474, 480, 486, 492, 498, 504, 510, 516, 522, 528 hours, or more. In some embodiments, cells can be cultured or expanded, for example, from about 1-96, 1-72, 1-48, 1-24, 1-12, 1-6, 1-3, 2-96, 2-72, 2-48, 2-24, 2-12, 2-6, 2-3, 3-96, 3-72, 3-78, 3-24, 3-12, or 3-6 hours.

In some embodiments, cells are cultured or expanded for about 48 hours. In some embodiments, cells are cultured or expanded for about 72 hours.

In some embodiments, conditions appropriate for T cell culture include an appropriate media (e.g., Minimal Essential Media or RPMI Media 1640 or, X-vivo 5, (Lonza)) that, in some embodiments, contains factors necessary for proliferation and viability, including serum (e.g., fetal bovine or human serum), interleukin-2 (IL-2), insulin, IFN-g, IL-4, IL-7, GM-CSF, IL-10, IL-21, IL-15, TGF beta, and TNF alpha or any other additives for the growth of cells. Other additives for the growth of cells include, but are not limited to, surfactant, plasmanate, S-2-hydroxyglutarate, and reducing agents such as N-acetyl-cysteine and 2-mercaptoethanol. Media can include RPMI 1640, A1 M-V, DMEM, MEM, α-MEM, F-12, X-Vivo 1, and X-Vivo 15, X-Vivo 20, Optimizer, with added amino acids, sodium pyruvate, and vitamins, either serum-free or supplemented with an appropriate amount of serum (or plasma) or a defined set of hormones, and/or an amount of cytokine(s) sufficient for the growth and expansion of T cells. In some embodiments, antibiotics, e.g., penicillin and streptomycin, are included in experimental cultures. In some embodiments, antibiotics, e.g., penicillin and streptomycin, are included in cultures of cells that are to be infused into a subject. In some embodiments, antibiotics, e.g., penicillin and streptomycin, are not included in cultures of cells that are to be infused into a subject. In some embodiments, the target cells are maintained under conditions necessary to support growth; for example, an appropriate temperature (e.g., 37° C.) and atmosphere (e.g., air plus 5% CO₂). In some instances, T cells that have been exposed to varied stimulation times may exhibit different characteristics. In some embodiments, antigens or antigen-binding fragments specific for CD3, CD28, CD2, or any combination thereof are used. In some embodiments, a soluble tetrameric antibody against human CD3, CD28, CD2, or any combination thereof is used.

In some embodiments, cells of the disclosure are expanded before or after other processes as described herein, for example, before or after gene disruption, before or after transgene introduction, before or after enrichment, or any combination thereof.

In some embodiments, cells of the disclosure are cryopreserved before or after expansion, and subsequently thawed and revived for further use (e.g., for gene disruption, transgene introduction, co-culture assay, functional evaluation, or a combination thereof). For example, in some embodiments, cells are cryopreserved prior to expansion, then thawed and expanded as described herein. In some embodiments, cells are expanded as described herein, then cryopreserved. In some embodiments, cells are cryopreserved, subsequently thawed and expanded as described herein, and the expanded cells can be cryopreserved. Cells can be cryopreserved using, for example, dimethyl sulfoxide (DMSO) as a cryoprotectant. Cells can be cryopreserved in media or buffer comprising, for example, about 5%, 6%, 7%, 8%, 9%, 10%, 11%, 12%, 13%, 14%, 15%, 16%, 17%, 18%, 19%, 20%, 21%, 21%, 22%, 23%, or 25% DMSO. In one embodiment, cells are cryopreserved in media comprising about 90% fetal bovine serum and about 10% DMSO.

### Introduction of Transgenes

An important part of T cell function is T cell receptor (TCR) recognition of a cognate antigen presented by MHC-I or MHC-II. Recognition of cognate antigen can lead to activation, proliferation, and effector functions (e.g., killing of target cells, production of inflammatory cytokines). To facilitate screens for immunomodulatory genes that involve T cell recognition of cognate antigen, transgenes can be used to introduce an exogenous TCR of known specificity into T cells.

a. Immunomodulatory Transgenes

In some embodiments, said transgene is an immunomodulatory transgene. In some embodiments, said immunomodulatory transgene encodes a protein that alters a function of an immune cell. In some embodiments, said immunomodulatory transgene encodes a protein that enhances or improves an immune function of an immune cell. In some embodiments, said immunomodulatory transgene encodes a protein that downregulates or inhibits an immune function of an immune cell. In some embodiments, said immunomodulatory transgene encodes a protein that alters a function of a T cell. In some embodiments, said immunomodulatory transgene encodes a protein that enhances or improves a function of a T cell. In some embodiments, said immunomodulatory transgene encodes a protein that downregulates or inhibits a function of a T cell. In some embodiments, said immunomodulatory transgene encodes a protein that improves a function of a T cell, wherein said protein is phosphodiesterase 1C (PDE1C), rhotekin 2 (RTKN2), nerve growth factor receptor (NGFR), or thymocyte-expressed molecule involved in selection (THEMIS).

In some embodiments, said transgene is randomly inserted into the genome. In some embodiments, insertion of said transgene is targeted to a pre-selected genomic locus. In some embodiments, said genomic locus is a safe harbor site. In some embodiments, said safe harbor site is an AAVS site (e.g., AAVS1, AAVS2), CCR5, hROSA26. In some embodiments, said genomic locus encodes a protein that negatively regulates an immune function or immune response of an immune cell. In some embodiments, said genomic locus encodes a protein that negatively regulates an immune function or immune response of a T cell. In some embodiments, said protein that negatively regulates an immune function or immune response of a T cell is an immune checkpoint gene. In some embodiments, said immune check point gene is PD1, CTLA-4, TCRA, TRAC, adenosine A2a receptor (ADORA), CD276, V-set domain containing T cell activation inhibitor 1 (VTCN1), B and T lymphocyte associated (BTLA), indoleamine 2,3-dioxygenase 1 (IDO1), killer cell immunoglobulin-like receptor, three domains, long cytoplasmic tail, 1 (KIR3DL1), lymphocyte-activation gene 3 (LAG3), hepatitis A virus cellular receptor 2 (HAVCR2), V-domain immunoglobulin suppressor of T-cell activation (VISTA), natural killer cell receptor 2B4 (CD244), hypoxanthine phosphoribosyltransferase 1 (HPRT), adeno-associated virus integration site 1 (AAVS1), or chemokine (C—C motif) receptor 5 (gene/pseudogene) (CCR5), CD160 molecule (CD160), T-cell immunoreceptor with Ig and ITIM domains (TIGIT), CD96 molecule (CD96), cytotoxic and regulatory T-cell molecule (CRTAM), leukocyte associated immunoglobulin like receptor 1 (LAIR1), sialic acid binding Ig like lectin 7 (SIGLEC7), sialic acid binding Ig like lectin 9 (SIGLEC9), tumor necrosis factor receptor superfamily member 10b (TNFRSF10B), tumor necrosis factor receptor superfamily member 10a (TNFRSF10A), caspase 8 (CASP8), caspase 10 (CASP10), caspase 3 (CASP3), caspase 6 (CASP6), caspase 7 (CASP7), Fas associated via death domain (FADD), Fas cell surface death receptor (FAS), transforming growth factor beta receptor II (TGFBRII), transforming growth factor beta receptor I (TGFBR1), SMAD family member 2 (SMAD2), SMAD family member 3 (SMAD3), SMAD family member 4 (SMAD4), SKI proto-oncogene (SKI), SKI-like proto-oncogene (SKIL), TGFB induced factor homeobox 1 (TGIF1), programmed cell death 1 (PD-1), cytotoxic T-lymphocyte-associated protein 4 (CTLA4), interleukin 10 receptor subunit alpha (IL10RA), interleukin 10 receptor subunit beta (IL10RB), heme oxygenase 2 (HMOX2), interleukin 6 receptor (IL6R), interleukin 6 signal transducer (IL6ST), c-src tyrosine kinase (CSK), phosphoprotein membrane anchor with glycosphingolipid microdomains 1 (PAG1), signaling threshold regulating transmembrane adaptor 1 (SIT1), forkhead box P3 (FOXP3), PR domain 1 (PRDM1), basic leucine zipper transcription factor, ATF-like (BATF), guanylate cyclase 1, soluble, alpha 2 (GUCY1A2), guanylate cyclase 1, soluble, alpha 3 (GUCY1A3), guanylate cyclase 1, soluble, beta 2 (GUCY1B2), prolyl hydroxylase domain (PHD1, PHD2, PHD3) family of proteins, or guanylate cyclase 1, soluble, beta 3 (GUCY1B3), egl-9 family hypoxia-inducible factor 1 (EGLN1), egl-9 family hypoxia-inducible factor 2 (EGLN2), egl-9 family hypoxia-inducible factor 3 (EGLN3), protein phosphatase 1 regulatory subunit 12C (PPP1R12C), NAD-dependent deacetylase sirtuin 2 (SIRT2), or Protein Tyrosine Phosphatase Non-Receptor Type 1 (PTPN1). In some embodiments, insertion of said transgene into said genomic locus that encodes a protein that negatively regulates an immune function or immune response of a T cell downregulates or completely inhibits expression of a functional protein encoded by said genomic locus. In some embodiments, insertion of said transgene into said genomic locus that encodes an immune check point gene downregulates or completely inhibits expression of a functional immune checkpoint protein.

In some embodiments, instead of or in addition to insertion of a transgene, expression of an endogenous gene is enhanced. In some embodiments, said endogenous gene encodes a protein that enhances or improves a function of an immune cell. In some embodiments, said endogenous gene encodes a protein that enhances or improves a function of a T cell. In some embodiments, said endogenous gene encodes is phosphodiesterase 1C (PDE1C), rhotekin 2 (RTKN2), nerve growth factor receptor (NGFR), or thymocyte-expressed molecule involved in selection (THEMIS). In some embodiments, said enhanced expression is mediated by CRISPR activation (CRISPRa). CRISPRa mediates enhanced expression by use of a gRNA that binds to or in proximity of a gene's promoter region or transcriptional start site.

b. T Cell Receptor (TCR) and Chimeric Antigen Receptor (CAR)

In some embodiments, a T cell comprises one or more transgenes. In some embodiments, the one or more transgenes express a TCR alpha, beta, gamma, and/or delta chain protein, and the TCR recognizes an epitope from a known antigen (e.g., G12D KRAS).

In some embodiments, a TCR comprises an alpha chain and beta chain sequence as defined herein. In some embodiments, a TCR comprises a gamma chain and a delta chain sequence as defined herein.

In some embodiments, a TCR comprises a fusion protein that maintains at least substantial biological activity. In some embodiments, in the case of the alpha and beta chain of a TCR, this can mean that both chains remain able to form a T cell receptor (either with a non-modified alpha and/or beta chain or with another fusion protein alpha and/or beta chain) which exerts its biological function, in particular binding to a specific peptide-MHC complex of a TCR, and/or functional signal transduction upon activation. In some embodiments, in the case of the gamma and delta chain of a TCR, this means that both chains remain able to form a T cell receptor (either with a non-modified gamma and/or delta chain or with another fusion protein gamma and/or delta chain) which exerts its biological function, in particular binding to a specific peptide-MHC complex or other ligand of the TCR, and/or functional signal transduction upon ligand recognition.

In some embodiments, a T cell comprises one or more TCRs. In some embodiments, a T cell comprises a single TCR specific to more than one target.

In some embodiments, a transgene (e.g., TCR gene) is inserted in a safe harbor locus. A safe harbor locus comprises a genomic location where a transgene can integrate and function without perturbing endogenous activity. For example, in some embodiments, one or more transgenes are inserted into any one of HPRT, AAVS SITE (E.G. AAVS1, AAVS2, ETC.), CCR5, hROSA26, and/or any combination thereof. In some embodiments, a transgene (e.g., TCR gene) is inserted in an endogenous immunomodulatory gene. In some embodiments, an endogenous immunomodulatory gene is a stimulatory immunomodulatory gene or an inhibitory immunomodulatory gene. In some embodiments, a transgene (e.g., TCR gene) is inserted in a stimulatory immunomodulatory gene such as CD27, CD40, CD122, OX40, GITR, CD137, CD28, or ICOS. Immunomodulatory gene locations can be provided using the Genome Reference Consortium Human Build 38 patch release 2 (GRCh38.p2) assembly. In some embodiments, a transgene (e.g., TCR gene) is inserted in an endogenous inhibitory immunomodulatory gene such as A2AR, B7-H3, B7-H4, BTLA, CTLA-4, IDO, KIR, LAG3, PD-1, TIM-3, VISTA, or CISH. In some embodiments, for example, one or more transgene is inserted into any one of CD27, CD40, CD122, OX40, GITR, CD137, CD28, ICOS, A2AR, B7-H3, B7-H4, BTLA, CTLA-4, IDO, KIR, LAG3, PD-1, TIM-3, VISTA, HPRT, AAVS SITE (E.G. AAVS1, AAVS2, ETC.), PHD1, PHD2, PHD3, CCR5, CISH, PPP1R12C, SIRT2, PTPN1, and/or any combination thereof. In some embodiments, a transgene is inserted in an endogenous TCR gene, for example, TRAC or TRB. In some embodiments, a transgene is inserted within a coding genomic region. In some embodiments, a transgene is inserted within a noncoding genomic region. In some embodiments, a transgene is inserted into a genome without homologous recombination. In some embodiments, a transgene is inserted into an AAV integration site. In some embodiments, an AAV integration site is a safe harbor in some cases. Alternative AAV integration sites may exist, such as AAVS2 on chromosome 5 or AAVS3 on chromosome 3. Additional AAV integration sites such as AAVS 2, AAVS3, AAVS4, AAVS5, AAVS6, AAVS7, AAVS8, and the like are also considered to be possible integration sites for an exogenous receptor, such as a TCR. As used herein, AAVS can refer to AAVS1 as well as related adeno-associated virus (AAVS) integration sites.

In some embodiments, a chimeric antigen receptor (CAR) is comprised of an extracellular antigen recognition domain, a trans-membrane domain, and a signaling region that controls T cell activation. In some embodiments, the extracellular antigen recognition domain is derived from a murine, humanized, or fully human monoclonal antibody. In some embodiments, the extracellular antigen recognition domain is comprised of the variable regions of the heavy and light chains of a monoclonal antibody that is cloned in the form of single-chain variable fragments (scFv) and joined through a hinge and a transmembrane domain to an intracellular signaling domain of the T-cell receptor (TCR) complex and at least one domain from a co-stimulatory molecule. In some embodiments, said CAR comprises a co-stimulatory domain. In some embodiments, said CAR does not contain a co-stimulatory domain.

In some embodiments, a CAR of the present disclosure is present in the plasma membrane of a eukaryotic cell, e.g., a mammalian cell, where suitable mammalian cells include, but are not limited to, a cytotoxic cell, a T lymphocyte, a stem cell, a progeny of a stem cell, a progenitor cell, a progeny of a progenitor cell, an NK cell, and an NKT cell. In some embodiments, when present in the plasma membrane of a eukaryotic cell, a CAR is active in the presence of its binding target. In some embodiments, a target is expressed on a membrane. In some embodiments, a target is soluble (e.g., not bound to a cell). In some embodiments, a target is present on the surface of a cell such as a target cell. In some embodiments, a target is presented on a solid surface such as a lipid bilayer; and the like. In some embodiments, a target is soluble, such as a soluble antigen. In some embodiments, a target is an antigen. In some embodiments, an antigen is present on the surface of a cell such as a target cell. In some embodiments, an antigen is presented on a solid surface such as a lipid bilayer; and the like. In some embodiments, a target is an epitope of an antigen. In some embodiments, a target is a cancer neo-antigen.

c. Site-Specific Insertion

In some embodiments, inserting one or more transgenes in any of the methods disclosed herein is site-specific. In some embodiments, a transgene comprises a promoter (for example, an MND promoter). In some embodiments, a transgene is inserted so as to utilize a promoter already present in the genome. For example, in some embodiments, one or more transgenes with promoters (e.g., a TCR) are inserted into the genome. In some embodiments, one or more transgenes lacking promoters (e.g., a TCR) are inserted adjacent to or near a promoter. In some embodiments, a transgene lacking a promoter utilizes, for example, a splice acceptor for insertion into a target sequence. In some embodiments, one or more transgenes are inserted adjacent to, near, or within an exon of a gene (e.g., TRAC). Such insertions can be used to knock-in a transgene (e.g., TCR transgene of known antigen specificity, such as a TCR transgene specific for G12D KRAS) while simultaneously disrupting an endogenous gene (e.g., TRAC). In another example, one or more transgenes can be inserted adjacent to, near, or within an intron of a gene. In some embodiments, a transgene is introduced using an AAV viral vector and integrated into a targeted genomic location. In some embodiments, a transgene (such as a TCR) is inserted into a TRAC locus or a TCRB locus. In some embodiments, a transgene (such as a TCR) is inserted into an immune checkpoint gene such as CISH, CTLA-4, and/or PD-1. By inserting a TCR into an immune checkpoint gene, an immune cell (such as a T cell) can be assayed for genes that have a synergistic cytotoxic (cytolytic) effect on cancer cells. For example, T cells can be assayed for synergy between a disruption in CISH and disruption in a second (candidate) gene.

In some embodiments, a transgene to be inserted is flanked by engineered sites analogous to a targeted double strand break site in the genome to excise the transgene from a polynucleic acid so it can be inserted at the double strand break region. In some embodiments, a transgene is virally introduced. For example, an AAV virus can be utilized to deliver a transgene to a cell. In some embodiments, a modified or engineered AAV virus is used to introduce a transgene to a cell. In some embodiments, a modified or wildtype AAV comprises homology arms to at least one genomic location.

Site specific gene editing can be achieved using non-viral gene editing such as CRISPR, TALEN (see U.S. Pat. No. 9,393,257), Argonaute (e.g., Argonaute systems capable of genomic disruption at mesophilic temperatures), transposon-based, Zinc Finger (ZFN), meganuclease, or Mega-TAL, or Transposon-based system. For example, PiggyBac (see Moriarty, B. S., et al., “Modular assembly of transposon integratable multigene vectors using RecWay assembly,” Nucleic Acids Research (8):e92 (2013) or sleeping beauty (see Aronovich, E. L, et al., “The Sleeping Beauty transposon system: a non-viral vector for gene therapy,” Hum. Mol. Genet., 20(R1): R14-R20. (2011) transposon systems can be used. In some embodiments, site specific gene editing is done with a two part system, for example, comprising a targeting moiety (e.g., a catalytically dead Cas protein such as dCas9) and a disrupting moiety (e.g., an endonuclease, such as a ZFN, TALEN, or Argonaute that is active at mesophilic temperatures). In this regard, the skilled worker will appreciate that although a CRISPR/Cas system provides certain advantages with respect to disruption efficiency, the various systems described herein also can be used to generate disruptions in candidate genes.

Site specific gene editing can also be achieved without homologous recombination. An exogenous polynucleic acid can be introduced into a cell genome without the use of homologous recombination. In some cases, a transgene can be flanked by engineered sites that are complementary to a targeted double strand break region in a genome. A transgene can be excised from a polynucleic acid so it can be inserted at a double strand break region without homologous recombination.

In some embodiments, a transgene is flanked by one or more engineered sites that are complementary to a targeted double strand break region in a genome. In some embodiments, engineered sites are not recombination arms. In some embodiments, engineered sites have homology to a double strand break region. In some embodiments, engineered sites have homology to a gene. In some embodiments, engineered sites have homology to a coding genomic region. In some embodiments, engineered sites have homology to a non-coding genomic region. In some embodiments, a transgene is excised from a polynucleic acid so it can be inserted at a double strand break region without homologous recombination. In some embodiments, a transgene integrates into a double strand break without homologous recombination.

In some embodiments, a transgene comprises a different sequence to the genomic sequence where it is placed. In some embodiments, a donor transgene contains a non-homologous sequence flanked by two regions of homology to allow for efficient homology-directed repair (HDR) at the location of interest. In some embodiments, a transgene is flanked by recombination arms. In some embodiments, recombination arms comprise complementary regions that target a transgene to a desired integration site. Additionally, In some embodiments, transgene sequences comprise a vector molecule containing sequences that are not homologous to the region of interest in cellular chromatin. In some embodiments, a transgene contains several, discontinuous regions of homology to cellular chromatin. For example, for targeted insertion of sequences not normally present in a region of interest, a sequence can be present in a donor nucleic acid molecule and flanked by regions of homology to sequence in the region of interest. In some embodiments, a transgene comprises a splice acceptor.

In some embodiments, a polynucleic acid comprises a transgene. In some embodiments, the transgene encodes an exogenous receptor. For example, In some embodiments, disclosed herein is a polynucleic acid comprising at least one exogenous T cell receptor (TCR) sequence flanked by at least two recombination arms having a sequence complementary to polynucleotides within a genomic sequence that is TRAC, adenosine A2a receptor, CD276, V-set domain containing T cell activation inhibitor 1, B and T lymphocyte associated, cytotoxic T-lymphocyte-associated protein 4, indoleamine 2,3-dioxygenase 1, killer cell immunoglobulin-like receptor, three domains, long cytoplasmic tail, 1, lymphocyte-activation gene 3, programmed cell death 1, hepatitis A virus cellular receptor 2, V-domain immunoglobulin suppressor of T-cell activation, or natural killer cell receptor 2B4.

### d Random Insertion

In some embodiments, one or more transgenes of the methods described herein are inserted randomly into the genome of a cell. These transgenes can be functional if inserted anywhere in a genome. For instance, a transgene can encode its own promoter or can be inserted into a position where it is under the control of an endogenous promoter. Alternatively, a transgene can be inserted into a gene, such as an intron of a gene, an exon of a gene, a promoter, or a non-coding region.

A nucleic acid, e.g., RNA, encoding a transgene sequences can be randomly inserted into a chromosome of a cell. A random integration can result from any method of introducing a nucleic acid, e.g., RNA, into a cell. For example, the method can be, but is not limited to, electroporation, sonoporation, use of a gene gun, lipotransfection, calcium phosphate transfection, use of dendrimers, microinjection, and use of viral vectors including adenoviral, AAV, and retroviral vectors, and/or group II ribozymes.

e. Transgene Expression, Composition, and Origin

A transgene can be used to express a gene of interest. In some embodiments, a transgene is for overexpression of an endogenous gene. In some embodiments, a transgene is used for expression of an exogenous gene, e.g. a gene that was not present in the genome prior to transgene introduction. Transgenes can also encompass other types of genes, for example, a dominant negative gene.

In some embodiments, a polynucleic acid vector comprising a transgene comprises a transgene promoter to facilitate expression of the transgene. In some embodiments, a polynucleic acid vector comprising a transgene lacks a transgene promoter, for example, resulting in expression of the transgene only when integrated into the genome at a location that comprises an upstream promoter, or within an open reading frame sequence comprising an upstream promoter. Use of a polynucleic acid vector comprising a transgene and lacking a transgene promoter can, for example, result in decreased episomal expression of the transgene, allow selection of cells comprising transgene integration and expression, or a combination thereof.

In some embodiments, a nucleic acid encoding a transgene is designed to include a reporter gene so that the presence of a transgene or its expression product can be detected via activation of the reporter gene. Any reporter gene can be used, such as a fluorescent protein (e.g. green fluorescent protein, GFP) or luciferase. Cells comprising a transgene can be selected based on expression of the reporter gene.

Expression of a transgene can be verified by an expression assay, for example, qPCR or by measuring levels of RNA. Expression level can be indicative also of copy number. For example, if expression levels are extremely high, this can indicate that more than one copy of a transgene was integrated in a genome. Alternatively, high expression can indicate that a transgene was integrated in a highly transcribed area, for example, near a highly expressed promoter. Expression can also be verified by measuring protein levels, such as through Western blotting. In some cases, a splice acceptor assay can be used with a reporter system to measure transgene integration.

A transgene polynucleic acid can be DNA or RNA, single-stranded or double-stranded and can be introduced into a cell in linear or circular form. A transgene sequence(s) can be contained within a DNA mini-circle, which may be introduced into the cell in circular or linear form. If introduced in linear form, the ends of a transgene sequence can be protected (e.g., from exonucleolytic degradation) by any method. For example, one or more dideoxynucleotide residues can be added to the 3′ terminus of a linear molecule and/or self-complementary oligonucleotides can be ligated to one or both ends. Additional methods for protecting exogenous polynucleotides from degradation include, but are not limited to, addition of terminal amino group(s) and the use of modified internucleotide linkages such as, for example, phosphorothioates, phosphoramidates, and O-methyl ribose or deoxyribose residues.

Generally, a transgene refers to a linear polymer comprising multiple nucleotide subunits. A transgene may comprise any number of nucleotides. In some cases, a transgene may comprise less than about 100 nucleotides. In some cases, a transgene may comprise at least about 100 nucleotides. In some cases, a transgene may comprise at least about 200 nucleotides. In some cases, a transgene may comprise at least about 300 nucleotides. In some cases, a transgene may comprise at least about 400 nucleotides. In some cases, a transgene may comprise at least about 500 nucleotides. In some cases, a transgene may comprise at least about 1000 nucleotides. In some cases, a transgene may comprise at least about 5000 nucleotides. In some cases, a transgene may comprise at least about 10,000 nucleotides. In some cases, a transgene may comprise at least about 20,000 nucleotides. In some cases, a transgene may comprise at least about 30,000 nucleotides. In some cases, a transgene may comprise at least about 40,000 nucleotides. In some cases, a transgene may comprise at least about 50,000 nucleotides. In some cases, a transgene may comprise between about 500 and about 5000 nucleotides. In some cases, a transgene may comprise between about 5000 and about 10,000 nucleotides. In any of the cases disclosed herein, the transgene may comprise DNA, RNA, or a hybrid of DNA and RNA. In some cases, the transgene may be single stranded. In some cases, the transgene may be double stranded.

One or more transgenes can be from different species. For example, one or more transgenes can comprise a human gene, a mouse gene, a rat gene, a pig gene, a bovine gene, a dog gene, a cat gene, a monkey gene, a chimpanzee gene, or any combination thereof. For example, a transgene can be from a human, having a human genetic sequence. One or more transgenes can comprise human genes. In some cases, one or more transgenes are not adenoviral genes.

f. Experimental Considerations for Transgene Introduction

Transgene(s) of the disclosure can be delivered to any number of cells. Transgenes can be delivered to, for example, about 1×10³, 2×10³, 3×10³, 4×10³, 5×10³, 6×10³, 7×10³, 8×10³, 9×10³, 1×10⁴, 2×10⁴, 3×10⁴, 4×10⁴, 5×10⁴, 6×10⁴, 7×10⁴, 8×10⁴, 9×10⁴, 1×10⁵, 2×10⁵, 3×10⁵, 4×10⁵, 5×10⁵, 6×10⁵, 7×10⁵, 8×10⁵, 9×10⁵, 1×10⁶, 2×10⁶, 3×10⁶, 4×10⁶, 5×10⁶, 6×10⁶, 7×10⁶, 8×10⁶, 9×10⁶, 1×10⁷, 2×10⁷, 3×10⁷, 4×10⁷, 5×10⁷, 6×10⁷, 7×10⁷, 8×10⁷, 9×10⁷, 1×10⁸, 2×10⁸, 3×10⁸, 4×10⁸, 5×10⁸, 6×10⁸, 7×10⁸, 8×10⁸, 9×10⁸, 1×10⁹, 2×10⁹, 3×10⁹, 4×10⁹, 5×10⁹, 6×10⁹, 7×10⁹, 8×10⁹, or 9×10⁹ cells.

In some embodiments, a transgene can be delivered by transduction with a viral vector. In some embodiments, a transgene can be delivered by a retrovirus, such as a lentiviral vector. In some embodiments, a transgene can be delivered by an adenovirus, parvovirus (e.g., adeno-associated virus (AAV)), retrovirus, herpesvirus, or integrase-defective lentivirus (IDLV). A viral vector can be used to deliver a transgene to target cells at a multiplicity of infection of, for example, about 5×10¹⁰:1, 1×10¹⁰:1, 5×10⁹:1, 1×10⁹:1, 5×10⁸:1, 1×10⁸:1, 5×10⁷:1, 1×10⁷:1, 5×10⁶:1, 1×10⁶:1, 5×10⁵:1, 1×10⁵:1, 5×10⁴:1, 1×10⁴:1, 5×10³:1, 1×10³:1, 900:1, 800:1, 700:1, 600:1, 500:1, 400:1, 300:1, 250:1, 200:1, 150:1, 100:1, 90:1, 80:1, 70:1, 60:1, 50:1, 40:1, 30:1, 20:1, 10:1, 5:1, 4:1, 3:1, 2:1, 1:1, 1:2, 1:3, 1:4, 1:5, 1:10, 1:20, 1:30, 1:40, 1:50, 1:60, 1:70, 1:80, 1:90, 1:100, 1:150, 1:200, 1:250, 1:300, 1:400, 1:500, 1:600, 1:700, 1:800, 1:900, 1:1×10³, 1:5×10³, 1:1×10⁴, 1:5×10⁴, 1:1×10⁵, 1:5×10⁵, 1:1×10⁶, 1:5×10⁶, 1:1×10⁷, 1:5×10⁷, 1:1×10⁸, 1:5×10⁸, 1:1×10⁹, 1:5×10⁹, 1:1×10¹⁰, or 1:5×10¹⁰. In some embodiments, a transgene can be delivered via electroporation.

After transgene introduction, cells of the disclosure can be allowed to recover prior to subsequent processing. For example, after transgene introduction, cells can be recovered by culturing in complete media prior to expansion, stimulation, enrichment, cryopreservation, co-culture assays, or functional evaluation. Cells can be recovered after transgene introduction, for example, for about 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 102, 108, 114, 120, 126, 132, 138, 144, 150, 156, 162, 168, 174, 180, 186, 192, 198, 204, 210, 216, 222, 228, 234, 240, 246, 252, 258, 264, 270, 276, 282, 288, 294, 300, 306, 312, 318, 324, 330, 336, 342, 348, 354, 360, 366, 372, 378, 384, 390, 396, 402, 408, 414, 420, 426, 432, 438, 444, 450, 456, 462, 468, 474, 480, 486, 492, 498, 504, 510, 516, 522, 528 hours, or more prior to subsequent processing.

Cells of the disclosure can be cryopreserved before or after transgene introduction. For example, cells can be cryopreserved, then thawed, cultured, and transgene(s) introduced as described herein. Transgene(s) can be introduced to cells as described herein, and cells comprising introduced transgenes can subsequently be cryopreserved. Cells can be cryopreserved, subsequently thawed and subjected to transgene introduction, and cryopreserved after transgene introduction. After thawing, cells can be recovered in media prior to subsequent use.

### Gene Disruption

Disclosed herein are, inter alia, methods for identifying immunomodulatory genes. To screen for immunomodulatory genes in T cells, gene disruption (knockout) techniques can be used, and the effects of gene disruption on T cell function can be tested (for example, a cytotoxicity assay for killing of target cells). Gene disruption techniques can also be used to disrupt known immunomodulatory genes, for example, to serve as controls, or to look for additive effects. Further, gene disruption techniques can be used to facilitate transgene integration into a desired location in the genome (e.g., integration of a TCR specific for G12D KRAS into the TRAC locus.

In some embodiments, gene disruption techniques comprise gene editing. For example, gene editing can be performed using a nuclease, including CRISPR associated proteins (Cas proteins, e.g., Cas9), Zinc finger nuclease (ZFN), Transcription Activator-Like Effector Nuclease (TALEN), Argonaute nucleases, and meganucleases. Nucleases include, but are not limited to, naturally existing nucleases, genetically modified, and/or recombinant. Gene editing can also be performed using a transposon-based system (e.g. PiggyBac, Sleeping beauty). For example, in some embodiments, gene editing is performed using a transposase.

In some embodiments, a CRISPR system is used to generate a double stranded break in a target gene in order to disrupt a candidate immunomodulatory gene, facilitate integration of a transgene, or a combination thereof. In some embodiments, a CRISPR-associated (Cas) protein comprises an enzymatic activity to generate a double-stranded break (DSB) in DNA, at a site determined by a guide RNA (gRNA). Site-specific cleavage of a target DNA occurs at locations determined by both 1) base-pairing complementarity between the guide RNA and the target DNA (also called a protospacer) and 2) a short motif in the target DNA referred to as the protospacer adjacent motif (PAM). For example, an engineered cell can be generated using a CRISPR system, e.g., a type II CRISPR system. A Cas enzyme used in the methods disclosed herein can be Cas9, which catalyzes DNA cleavage. Enzymatic action by Cas9 derived from Streptococcus pyogenes or any closely related Cas9 can generate double stranded breaks at target site sequences which hybridize to 20 nucleotides of a guide sequence and that have a protospacer-adjacent motif (PAM) following the 20 nucleotides of the target sequence. CRISPR systems are described in greater detail elsewhere in the application.

Libraries or arrays of guide RNAs (gRNAs) can be used with a Cas protein to disrupt a plurality of genes. For example, an array of gRNAs can be used to disrupt a plurality of candidate immunomodulatory genes in T cells, and the effect of gene disruption on immune functions can be evaluated, for example, by measuring ability to kill target cells in a cytotoxicity assay.

In some embodiments, a T cell comprises one or more disrupted genes and one or more transgenes. In some embodiments, an endogenous TCR is disrupted, and a transgene encoding a TCR of known specificity is knocked in. In some embodiments, the TCR of known specificity can target a tumor antigen or neoantigen. In some embodiments, an endogenous TCR is disrupted, and a transgene encoding a TCR specific for G12D KRAS is knocked in.

In some embodiments, candidate immunomodulatory genes are genes that have not yet been identified as immunomodulatory genes. Accordingly, In some embodiments, an array of gRNAs comprises gRNAs targeting a plurality of genes, or even all genes in the genome. In some embodiments, the array of gRNAs comprises gRNAs targeting the druggable genome. In some embodiments, the array of gRNAs comprises gRNAs targeting a selection of target genes. In some embodiments, the array of gRNAs comprises gRNAs targeting genes selected by an algorithm as described herein.

In some embodiments, the gRNAs are delivered by transduction with a viral vector. In some embodiments, the gRNAs are delivered by a retrovirus, such as a lentiviral vector. In some embodiments, the gRNAs are delivered by an adenovirus, parvovirus (e.g., adeno-associated virus (AAV)), retrovirus, herpesvirus, or integrase-defective lentivirus (IDLV). In some embodiments, gRNAs are delivered via electroporation. In some embodiments, synthetic gRNAs incorporating RNA base modifications to confer resistance to enzymatic degradation within cells (such as 2′-O-methyl 3′ phosphorthioate incorporated onto single or multiple terminal RNA bases) are delivered via electroporation or other methods of transfection.

In some embodiments, known immunomodulatory genes are disrupted, for example, in combination with the disruption of one or more candidate genes. In some embodiments, known immunomodulatory gene locations are provided using the Genome Reference Consortium Human Build 38 patch release 2 (GRCh38.p2) assembly.

In some embodiments, a gene to be knocked out is selected using a database. In some cases, certain endogenous genes are more amendable to genomic engineering. In some embodiments, a database comprises epigenetically permissive target sites. A database can be ENCODE (encyclopedia of DNA Elements) (http://www.genome.gov/10005107) in some cases. In some embodiments, a database can identify regions with open chromatin that can be more permissive to genomic engineering.

For example, In some embodiments, one or more genes whose expression is disrupted comprise any one of adenosine A2a receptor (ADORA), CD276, V-set domain containing T cell activation inhibitor 1 (VTCN1), B and T lymphocyte associated (BTLA), cytotoxic T-lymphocyte-associated protein 4 (CTLA4), indoleamine 2,3-dioxygenase 1 (IDO1), killer cell immunoglobulin-like receptor, three domains, long cytoplasmic tail, 1 (KIR3DL1), lymphocyte-activation gene 3 (LAG3), programmed cell death 1 (PD-1), hepatitis A virus cellular receptor 2 (HAVCR2), V-domain immunoglobulin suppressor of T-cell activation (VISTA), natural killer cell receptor 2B4 (CD244), cytokine inducible 5H2-containing protein (CISH), hypoxanthine phosphoribosyltransferase 1 (HPRT), adeno-associated virus integration site (AAVS SITE (E.G. AAVS1, AAVS2, ETC.)), or chemokine (C—C motif) receptor 5 (gene/pseudogene) (CCR5), CD160 molecule (CD160), T-cell immunoreceptor with Ig and ITIM domains (TIGIT), CD96 molecule (CD96), cytotoxic and regulatory T-cell molecule (CRTAM), leukocyte associated immunoglobulin like receptor 1 (LAIR1), sialic acid binding Ig like lectin 7 (SIGLEC7), sialic acid binding Ig like lectin 9 (SIGLEC9), tumor necrosis factor receptor superfamily member 10b (TNFRSF10B), tumor necrosis factor receptor superfamily member 10a (TNFRSF10A), caspase 8 (CASP8), caspase 10 (CASP10), caspase 3 (CASP3), caspase 6 (CASP6), caspase 7 (CASP7), Fas associated via death domain (FADD), Fas cell surface death receptor (FAS), transforming growth factor beta receptor II (TGFBRII), transforming growth factor beta receptor I (TGFBR1), SMAD family member 2 (SMAD2), SMAD family member 3 (SMAD3), SMAD family member 4 (SMAD4), SKI proto-oncogene (SKI), SKI-like proto-oncogene (SKIL), TGFB induced factor homeobox 1 (TGIF1), interleukin 10 receptor subunit alpha (IL10RA), interleukin 10 receptor subunit beta (IL10RB), heme oxygenase 2 (HMOX2), interleukin 6 receptor (IL6R), interleukin 6 signal transducer (IL6ST), c-src tyrosine kinase (CSK), phosphoprotein membrane anchor with glycosphingolipid microdomains 1 (PAG1), signaling threshold regulating transmembrane adaptor 1 (SIT1), forkhead box P3 (FOXP3), PR domain 1 (PRDM1), basic leucine zipper transcription factor, ATF-like (BATF), guanylate cyclase 1, soluble, alpha 2 (GUCY1A2), guanylate cyclase 1, soluble, alpha 3 (GUCY1A3), guanylate cyclase 1, soluble, beta 2 (GUCY1B2), guanylate cyclase 1, soluble, beta 3 (GUCY1B3), cytokine inducible SH2-containing protein (CISH), prolyl hydroxylase domain (PHD1, PHD2, PHD3) family of proteins, NAD-dependent deacetylase sirtuin 2 (SIRT2), or Protein Tyrosine Phosphatase Non-Receptor Type 1 (PTPN1), or any combination thereof. For example, solely to illustrate various combinations, one or more genes whose expression is disrupted can comprise PD-1, CLTA-4, and CISH. In some embodiments, the expression of PD-1, CTLA-4, CISH, an additional candidate immunomodulatory gene, or some combination thereof is disrupted.

In some embodiments, one or more genes whose expression is disrupted comprise any one of CD27, CD40, CD122, OX40, GITR, CD137, CD28, ICOS, A2AR, B7-H3, B7-H4, BTLA, CTLA-4, IDO, KIR, LAG3, PD-1, TIM-3, PHD1, PHD2, PHD3, VISTA, CISH, PPP1R12C, SIRT2, PTPN1, or any combination thereof.

Examples of genes that can be disrupted include genes provided in Table 1.

TABLE 1
Disruption targets

| | | | NCBI number | | | |
|---|---|---|---|---|---|---|
| | | | (GRCh38.p2) | | | |
| Gene | | | *AC010327.8 | Original | Original | Location |
| Symbol | Abbreviation | Name | **GRCh38.p7 | Start | Stop | in genome |
| ADORA2A | A2aR; RDC8; | adenosine | 135 | 24423597 | 24442360 | 22q11.23 |
| | ADORA2 | A2a receptor | | | | |
| CD276 | B7H3; B7-H3; | CD276 molecule | 80381 | 73684281 | 73714518 | 15q23-q24 |
| | B7RP-2; 4Ig-B7-H3 | | | | | |
| VTCN1 | B7X; B7H4; B7S1; | V-set domain | 79679 | 117143587 | 117270368 | 1p13.1 |
| | B7-H4; B7h.5; | containing T | | | | |
| | VCTN1; PRO1291 | cell activation | | | | |
| | | inhibitor 1 | | | | |
| BTLA | BTLA1; CD272 | B and T | 151888 | 112463966 | 112499702 | 3q13.2 |
| | | lymphocyte | | | | |
| | | associated | | | | |
| CTLA4 | GSE; GRD4; | cytotoxic T- | 1493 | 203867788 | 203873960 | 2q33 |
| | ALPS5; CD152; | lymphocyte- | | | | |
| | CTLA-4; IDDM12; | associated | | | | |
| | CELIAC3 | protein 4 | | | | |
| IDO1 | IDO; INDO; IDO-1 | indoleamine | 3620 | 39913809 | 39928790 | 8p12-p11 |
| | | 2,3- | | | | |
| | | dioxygenase 1 | | | | |
| KIR3DL1 | KIR; NKB1; | killer cell | 3811 | 54816438 | 54830778 | 19q13.4 |
| | NKAT3; NKB1B; | immunoglobulin- | | | | |
| | NKAT-3; | like receptor, | | | | |
| | CD158E1; | three domains, | | | | |
| | KIR3DL2; | long cytoplasmic | | | | |
| | KIR3DL1/S1 | tail, 1 | | | | |
| LAG3 | LAG3; CD223 | lymphocyte- | 3902 | 6772483 | 6778455 | 12p13.32 |
| | | activation | | | | |
| | | gene 3 | | | | |
| PDCD1 | PD1; PD-1; | programmed | 5133 | 241849881 | 241858908 | 2q37.3 |
| | CD279; SLEB2; | cell death 1 | | | | |
| | hPD-1; hPD-l; | | | | | |
| | hSLE1 | | | | | |
| HAVCR2 | TIM3; CD366; | hepatitis A | 84868 | 157085832 | 157109237 | 5q33.3 |
| | KIM-3; TIMD3; | virus cellular | | | | |
| | Tim-3; TIMD-3; | receptor 2 | | | | |
| | HAVcr-2 | | | | | |
| VISTA | C10orf54, | V-domain | 64115 | 71747556 | 71773580 | 10q22.1 |
| | differentiation of | immunoglobulin | | | | |
| | ESC-1 (Dies1); | suppressor | | | | |
| | platelet receptor | of T-cell | | | | |
| | Gi24 precursor; | activation | | | | |
| | PD1 homolog | | | | | |
| | (PD1H) B7H5; | | | | | |
| | GI24; B7-H5; | | | | | |
| | SISP1; PP2135 | | | | | |
| CD244 | 2B4; 2B4; NAIL; | CD244 molecule, | 51744 | 160830158 | 160862902 | 1q23.3 |
| | Nmrk; NKR2B4; | natural killer | | | | |
| | SLAMF4 | cell receptor | | | | |
| | | 2B4 | | | | |
| CISH | CIS; G18; SOCS; | cytokine | 1154 | 50606454 | 50611831 | 3p21.3 |
| | CIS-1; BACTS2 | inducible | | | | |
| | | SH2- | | | | |
| | | containing | | | | |
| | | protein | | | | |
| HPRT1 | HPRT; HGPRT | hypoxanthine | 3251 | 134452842 | 134500668 | Xq26.1 |
| | | phosphoribo- | | | | |
| | | syltransferase | | | | |
| | | 1 | | | | |
| AAV*S1 | AAV | adeno- | 14 | 7774 | 11429 | 19q13 |
| | | associated | | | | |
| | | virus | | | | |
| | | integration | | | | |
| | | site 1 | | | | |
| CCR5 | CKR5; CCR-5; | chemokine | 1234 | 46370142 | 46376206 | 3p21.31 |
| | CD195; CKR-5; | (C-C motif) | | | | |
| | CCCKR5; | receptor 5 | | | | |
| | CMKBR5; | (gene/ | | | | |
| | IDDM22; CC- | pseudogene) | | | | |
| | CKR-5 | | | | | |
| CD160 | NK1; BY55; NK28 | CD160 molecule | 11126 | 145719433 | 145739288 | 1q21.1 |
| TIGIT | VSIG9; VSTM3; | T-cell | 201633 | 114293986 | 114310288 | 3q13.31 |
| | WUCAM | immunoreceptor | | | | |
| | | with Ig and | | | | |
| | | ITIM domains | | | | |
| CD96 | TACTILE | CD96 molecule | 10225 | 111542079 | 111665996 | 3q13.13-q13.2 |
| CRTAM | CD355 | cytotoxic and | 56253 | 122838431 | 122872643 | 11q24.1 |
| | | regulatory T- | | | | |
| | | cell molecule | | | | |
| LAIR1 | CD305; LAIR-1 | leukocyte | 3903 | 54353624 | 54370556 | 19q13.4 |
| | | associated | | | | |
| | | immunoglobulin | | | | |
| | | like receptor 1 | | | | |
| SIGLEC7 | p75; QA79; | sialic acid | 27036 | 51142294 | 51153526 | 19q13.3 |
| | AIRM1; CD328; | binding Ig | | | | |
| | CDw328; D-siglec; | like lectin 7 | | | | |
| | SIGLEC-7; | | | | | |
| | SIGLECP2; | | | | | |
| | SIGLEC19P; | | | | | |
| | p75/AIRM1 | | | | | |
| SIGLEC9 | CD329; CDw329; | sialic acid | 27180 | 51124880 | 51141020 | 19q13.41 |
| | FOAP-9; siglec-9; | binding Ig | | | | |
| | OBBP-LIKE | like lectin 9 | | | | |
| TNFRSF10B | DR5; CD262; | tumor | 8795 | 23006383 | 23069187 | 8p22-p21 |
| | KILLER; TRICK2; | necrosis | | | | |
| | TRICKB; | factor | | | | |
| | ZTNFR9; | receptor | | | | |
| | TRAILR2; | superfamily | | | | |
| | TRICK2A; | member 10b | | | | |
| | TRICK2B; | | | | | |
| | TRAIL-R2; | | | | | |
| | KILLER/DR5 | | | | | |
| TNFRSF10A | DR4; APO2; | tumor | 8797 | 23191457 | 23225167 | 8p21 |
| | CD261; TRAILR1; | necrosis | | | | |
| | TRAILR-1 | factor | | | | |
| | | receptor | | | | |
| | | superfamily | | | | |
| | | member 10a | | | | |
| CASP8 | CAP4; MACH; | caspase 8 | 841 | 201233443 | 201287711 | 2q33-q34 |
| | MCH5; FLICE; | | | | | |
| | ALPS2B; Casp-8 | | | | | |
| CASP10 | MCH4; ALPS2; | caspase 10 | 843 | 201182898 | 201229406 | 2q33-q34 |
| | FLICE2 | | | | | |
| CASP3 | CPP32; SCA-1; | caspase 3 | 836 | 184627696 | 184649475 | 4q34 |
| | CPP32B | | | | | |
| CASP6 | MCH2 | caspase 6 | 839 | 109688628 | 109713904 | 4q25 |
| CASP7 | MCH3; CMH-1; | caspase 7 | 840 | 113679162 | 113730909 | 10q25 |
| | LICE2; CASP-7; | | | | | |
| | ICE-LAP3 | | | | | |
| FADD | GIG3; MORT1 | Fas associated | 8772 | 70203163 | 70207402 | 11q13.3 |
| | | via death | | | | |
| | | domain | | | | |
| FAS | APT1; CD95; | Fas cell | 355 | 88969801 | 89017059 | 10q24.1 |
| | FAS1; APO-1; | surface death | | | | |
| | FASTM; ALPS1A; | receptor | | | | |
| | TNFRSF6 | | | | | |
| TGFBRII | AAT3; FAA3; | transforming | 7048 | 30606493 | 30694142 | 3p22 |
| | LDS2; MFS2; | growth factor | | | | |
| | RIIC; LDS1B; | beta receptor | | | | |
| | LDS2B; TAAD2; | II | | | | |
| | TGFR-2; TGFbeta- | | | | | |
| | RII | | | | | |
| TGFBR1 | AAT5; ALK5; | transforming | 7046 | 99104038 | 99154192 | 9q22 |
| | ESS1; LDS1; | growth factor | | | | |
| | MSSE; SKR4; | beta receptor I | | | | |
| | ALK-5; LDS1A; | | | | | |
| | LDS2A; TGFR-1; | | | | | |
| | ACVRLK4; | | | | | |
| | tbetaR-I | | | | | |
| SMAD2 | JV18; MADH2; | SMAD family | 4087 | 47833095 | 47931193 | 18q21.1 |
| | MADR2; JV18-1; | member 2 | | | | |
| | hMAD-2; | | | | | |
| | hSMAD2 | | | | | |
| SMAD3 | LDS3; LDS1C; | SMAD family | 4088 | 67065627 | 67195195 | 15q22.33 |
| | MADH3; JV15-2; | member 3 | | | | |
| | HSPC193; | | | | | |
| | HsT17436 | | | | | |
| SMAD4 | JIP; DPC4; | SMAD family | 4089 | 51030213 | 51085042 | 18q21.1 |
| | MADH4; MYHRS | member 4 | | | | |
| SKI | SGS; SKV | SKI proto- | 6497 | 2228695 | 2310213 | 1p36.33 |
| | | oncogene | | | | |
| SKIL | SNO; SnoA; SnoI; | SKI-like | 6498 | 170357678 | 170396849 | 3q26 |
| | SnoN | proto- | | | | |
| | | oncogene | | | | |
| TGIF1 | HPE4; TGIF | TGFB | 7050 | 3411927 | 3458411 | 18p11.3 |
| | | induced factor | | | | |
| | | homeobox 1 | | | | |
| IL10RA | CD210; IL10R; | interleukin 10 | 3587 | 117986391 | 118001483 | 11q23 |
| | CD210a; | receptor | | | | |
| | CDW210A; HIL- | subunit alpha | | | | |
| | 10R; IL-10R1 | | | | | |
| IL10RB | CRFB4; CRF2-4; | interleukin 10 | 3588 | 33266360 | 33297234 | 21q22.11 |
| | D21S58; D21S66; | receptor | | | | |
| | CDW210B; IL- | subunit beta | | | | |
| | 10R2 | | | | | |
| HMOX2 | HO-2 | heme | 3163 | 4474703 | 4510347 | 16p13.3 |
| | | oxygenase 2 | | | | |
| IL6R | IL6Q; gp80; | interleukin 6 | 3570 | 154405193 | 154469450 | 1q21 |
| | CD126; IL6RA; | receptor | | | | |
| | IL6RQ; IL-6RA; | | | | | |
| | IL-6R-1 | | | | | |
| IL6ST | CD130; GP130; | interleukin 6 | 3572 | 55935095 | 55994993 | 5q11.2 |
| | CDW130; IL-6RB | signal | | | | |
| | | transducer | | | | |
| CSK | CSK | c-src tyrosine | 1445 | 74782084 | 74803198 | 15q24.1 |
| | | kinase | | | | |
| PAG1 | CBP; PAG | phosphoprotein | 55824 | 80967810 | 81112068 | 8q21.13 |
| | | membrane | | | | |
| | | anchor with | | | | |
| | | glycosphingolipid | | | | |
| | | microdomains 1 | | | | |
| SIT1 | SIT1 | signaling | 27240 | 35649298 | 35650950 | 9p13-p12 |
| | | threshold | | | | |
| | | regulating | | | | |
| | | transmembrane | | | | |
| | | adaptor 1 | | | | |
| FOXP3 | JM2; AIID; IPEX; | forkhead box | 50943 | 49250436 | 49269727 | Xp11.23 |
| | PIDX; XPID; | P3 | | | | |
| | DIETER | | | | | |
| PRDM1 | BLIMP1; PRDI- | PR domain 1 | 639 | 106086320 | 106109939 | 6q21 |
| | BF1 | | | | | |
| BATF | SFA2; B-ATF; | basic leucine | 10538 | 75522441 | 75546992 | 14q24.3 |
| | BATF1; SFA-2 | zipper | | | | |
| | | transcription | | | | |
| | | factor, ATF- | | | | |
| | | like | | | | |
| GUCY1A2 | GC-SA2; GUC1A2 | guanylate | 2977 | 106674012 | 107018445 | 11q21-q22 |
| | | cyclase 1, | | | | |
| | | soluble, alpha | | | | |
| | | 2 | | | | |
| GUCY1A3 | GUCA3; MYMY6; | guanylate | 2982 | 155666568 | 155737062 | 4q32.1 |
| | GC-SA3; | cyclase 1, | | | | |
| | GUC1A3; | soluble, alpha | | | | |
| | GUCSA3; | 3 | | | | |
| | GUCY1A1 | | | | | |
| GUCY1B2 | GUCY1B2 | guanylate | 2974 | 50994511 | 51066157 | 13q14.3 |
| | | cyclase 1, | | | | |
| | | soluble, beta 2 | | | | |
| | | (pseudogene) | | | | |
| GUCY1B3 | GUCB3; GC-SB3; | guanylate | 2983 | 155758973 | 155807642 | 4q31.3-q33 |
| | GUC1B3; | cyclase 1, | | | | |
| | GUCSB3; | soluble, beta 3 | | | | |
| | GUCY1B1; GC-S- | | | | | |
| | beta-1 | | | | | |
| TRA | IMD7; TCRA; | T-cell receptor | 6955 | 21621904 | 22552132 | 14q11.2 |
| | TCRD; TRAalpha; | alpha locus | | | | |
| | TRAC | | | | | |
| TRB | TCRB; TRBbeta | T cell receptor | 6957 | 142299011 | 142813287 | 7q34 |
| | | beta locus | | | | |
| EGLN1 | HPH2; PHD2; | egl-9 family | 54583 | 231363751 | 231425044 | 1q42.1 |
| | SM20; ECYT3; | hypoxia- | | | | |
| | HALAH; HPH-2; | inducible | | | | |
| | HIFPH2; | factor 1 | | | | |
| | ZMYND6; | | | | | |
| | C1orf12; HIF-PH2 | | | | | |
| EGLN2 | EIT6; PHD1; | egl-9 family | 112398 | 40799143 | 40808441 | 19q13.2 |
| | HPH-1; HPH-3; | hypoxia- | | | | |
| | HIFPH1; HIF-PH1 | inducible | | | | |
| | | factor 2 | | | | |
| EGLN3 | PHD3; HIFPH3; | egl-9 family | 112399 | 33924215 | 33951083 | 14q13.1 |
| | HIFP4H3 | hypoxia- | | | | |
| | | inducible | | | | |
| | | factor 3 | | | | |
| PPP1R12C** | p84; p85; LENG3; | protein | 54776 | 55090913 | 55117600 | 19q13.42 |
| | MBS85 | phosphatase 1 | | | | |
| | | regulatory | | | | |
| | | subunit 12C | | | | |

In some embodiments, multiple genes are disrupted in one experiment. In some embodiments, multiple genes are disrupted in a single population of cells or in distinct populations of cells. In some embodiments, a different gene is disrupted in each well of a 96 well plate. In some embodiments, the number of different genes disrupted in a single experiment is greater than at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 105, 110, 115, 120, 125, 130, 135, 140, 145, 150, 160, 170, 180, 190, 192, 200, 250, 288, 300, 384, 400, 500, 600, 700, 800, 900, 1000, 2500, 3000, 4000, 5000, 6000, 7000, 8000, 9000, 10000, 15000, 20000, 25000, or 30000 genes. In some embodiments, the number of genes disrupted in a single experiment is at most about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 105, 110, 115, 120, 125, 130, 135, 140, 145, 150, 160, 170, 180, 190, 192, 200, 250, 288, 300, 384, 400, 500, 600, 700, 800, 900, 1000, 2500, 3000, 4000, 5000, 6000, 7000, 8000, 9000, 10000, 15000, 20000, 25000, or 30000 genes.

In some embodiments, one or more genes in a cell are knocked out or disrupted using any method. For example, one or more candidate immunomodulatory genes in a T cell can be knocked out or disrupted, and the resulting T cell can be functionally evaluated, e.g., by measuring ability to kill target cells in a cytotoxicity assay. In some embodiments, knocking out one or more genes comprises disrupting one or more genes from a genome of a T cell. In some embodiments, knocking out also comprises removing all or a part of a gene sequence from a T cell. In some embodiments, knocking out comprises replacing all or a part of a gene in a genome of a T cell with one or more nucleotides. In some embodiments, knocking out one or more genes comprises inserting a sequence in one or more genes thereby disrupting expression of the one or more genes. For example, in some embodiments, inserting a sequence can generate a stop codon in the middle of one or more genes. Inserting a sequence can also shift the open reading frame of one or more genes.

Gene disruption methods as disclosed herein can be applied to any number of cells. Gene disruption methods can be carried out utilizing, for example, about 1×10³, 2×10³, 3×10³, 4×10³, 5×10³, 6×10³, 7×10³, 8×10³, 9×10³, 1×10⁴, 2×10⁴, 3×10⁴, 4×10⁴, 5×10⁴, 6×10⁴, 7×10⁴, 8×10⁴, 9×10⁴, 1×10⁵, 2×10⁵, 3×10⁵, 4×10⁵, 5×10⁵, 6×10⁵, 7×10⁵, 8×10⁵, 9×10⁵, 1×10⁶, 2×10⁶, 3×10⁶, 4×10⁶, 5×10⁶, 6×10⁶, 7×10⁶, 8×10⁶, 9×10⁶, 1×10⁷, 2×10⁷, 3×10⁷, 4×10⁷, 5×10⁷, 6×10⁷, 7×10⁷, 8×10⁷, 9×10⁷, 1×10⁸, 2×10⁸, 3×10⁸, 4×10⁸, 5×10⁸, 6×10⁸, 7×10⁸, 8×10⁸, 9×10⁸, 1×10⁹, 2×10⁹, 3×10⁹, 4×10⁹, 5×10⁹, 6×10⁹, 7×10⁹, 8×10⁹, or 9×10⁹ target cells, or more.

In some embodiments, after gene disruption, cells of the disclosure are allowed to recover prior to subsequent processing. For example, in some embodiments, after gene disruption, cells are recovered by culturing in complete media prior to expansion, stimulation, enrichment, cryopreservation, co-culture assays, or functional evaluation. In some embodiments, cells are recovered after gene disruption, for example, for about 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 102, 108, 114, 120, 126, 132, 138, 144, 150, 156, 162, 168, 174, 180, 186, 192, 198, 204, 210, 216, 222, 228, 234, 240, 246, 252, 258, 264, 270, 276, 282, 288, 294, 300, 306, 312, 318, 324, 330, 336, 342, 348, 354, 360, 366, 372, 378, 384, 390, 396, 402, 408, 414, 420, 426, 432, 438, 444, 450, 456, 462, 468, 474, 480, 486, 492, 498, 504, 510, 516, 522, 528 hours, or more prior to subsequent processing.

In some embodiments, cells of the disclosure are cryopreserved before or after gene disruption. For example, in some embodiments, cells are cryopreserved, then thawed, cultured, and gene(s) disrupted as described herein. In some embodiments, gene(s) are disrupted in cells as described herein, and cells comprising disrupted genes are subsequently be cryopreserved. In some embodiments, cells are cryopreserved, subsequently thawed, subjected to gene disruption, and cryopreserved after gene disruption.

Gene suppression can also be done in a number of ways. For example, gene expression can be suppressed by knock out, altering a promoter of a gene, and/or by administering interfering RNAs. This can be done at an organism level or at a tissue, organ, and/or cellular level. If one or more genes are knocked down in a cell, tissue, and/or organ, the one or more genes can be suppressed by administrating RNA interfering reagents, e.g., siRNA, shRNA, or microRNA. For example, a nucleic acid which can express shRNA can be stably transfected into a cell to knockdown expression. Furthermore, a nucleic acid which can express shRNA can be inserted into the genome of a T cell, thus knocking down a gene within the T cell.

### Enrichment, Quality Control, and Storage of Edited Cells

Disclosed herein are, inter alia, screening assays to identify immunomodulatory genes. Having highly-enriched populations of desirable cells can contribute to the sensitivity of these assays. For example, populations comprising mostly T cells expressing a TCR of known specificity will facilitate greater recognition of target cells (e.g. in a cytotoxicity assay with cells expressing cognate antigen) compared to heterogeneous populations of T cells.

In some embodiments, cells comprising a gene disruption or transgene insertion are enriched, for example, using fluorescent activated cell sorting (FACS) with positive or negative selection, magnetic activated cell sorting (MACS) with positive or negative selection, culture based methods (e.g., selective expansion in culture, chemical selection (e.g., antibiotic resistance)), or a combination thereof. In some embodiments, gain or loss of reporter gene expression is the basis of enrichment (e.g., a fluorescent protein or luciferase).

In some embodiments, populations of cells comprising cells expressing a TCR of known specificity, or comprising a gene disruption of interest, are stained with fluorescently-conjugated antibodies or peptide-MHC multimers and sorted by FACS.

In some embodiments, populations of cells comprising cells expressing a TCR of known specificity are enriched for cells expressing the TCR by selective expansion in culture. For example, in some embodiments, a transgene encoding a TCR of known specificity is introduced into a population of cells as described in the disclosure. In some embodiments, cells expressing the TCR are selectively expanded using methods that specifically activate the introduced TCR, but not cells lacking the TCR. In some embodiments, the introduced TCR is activated, for example, via antibodies or fragments thereof that specifically bind the introduced TCR, peptide-MHC multimers that specifically bind the introduced TCR, single chain peptide-MHC multimers that specifically bind the introduced TCR, artificial antigen presenting cells that specifically bind the TCR, or antigen presenting cells that present cognate antigen to the introduced TCR.

In some embodiments, the introduced transgene encodes a TCR comprising a different amino acid sequence than the endogenous TCR. In some embodiments, the different amino acids are part of variable or constant regions of the TCR. In some embodiments, the different amino acids are the basis of selective expansion of the cells comprising the introduced TCR. In some embodiments, the introduced transgene comprises one or more amino acid sequences from a murine TCR that differ from a human TCR.

In some embodiments, selective expansion in culture further comprises activation of one or more co-receptors, e.g., CD28, ICOS, CD27, or 4-1BB (CD137). Selective expansion can further comprise a cytokine signal, e.g., IL-1α, IL-10, IL-2, IL-4, IL-5, IL-6, IL-7, IL-9, IL-10, IL-12, IL-13, IL-15, IL-17, IL-21, IL-23, TNF-α, IFN-γ or any combination thereof.

In some embodiments, selective expansion of cells as described herein comprises culturing cells, for example, for at least about 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 102, 108, 114, 120, 126, 132, 138, 144, 150, 156, 162, 168, 174, 180, 186, 192, 198, 204, 210, 216, 222, 228, 234, 240, 246, 252, 258, 264, 270, 276, 282, 288, 294, 300, 306, 312, 318, 324, 330, 336, 342, 348, 354, 360, 366, 372, 378, 384, 390, 396, 402, 408, 414, 420, 426, 432, 438, 444, 450, 456, 462, 468, 474, 480, 486, 492, 498, 504, 510, 516, 522, 528 hours, or more.

In some embodiments, selective expansion of cells as described herein comprises culturing cells, for example, for at most about 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 102, 108, 114, 120, 126, 132, 138, 144, 150, 156, 162, 168, 174, 180, 186, 192, 198, 204, 210, 216, 222, 228, 234, 240, 246, 252, 258, 264, 270, 276, 282, 288, 294, 300, 306, 312, 318, 324, 330, 336, 342, 348, 354, 360, 366, 372, 378, 384, 390, 396, 402, 408, 414, 420, 426, 432, 438, 444, 450, 456, 462, 468, 474, 480, 486, 492, 498, 504, 510, 516, 522, 528 hours, or less.

In some embodiments, selective expansion of cells as described herein comprise culturing cells, for example, for between about 6-240, 12-168, 24-168, 36-168, 48-168, 72-168, 96-168, 120-168, 144-168, 12-144, 24-144, 36-144, 48-144, 60-144, 72-144, 96-144, 120-144, 12-120, 24-120, 36-120, 48-120, 60-120, 72-120, 84-120, 96-120, 108-120, 12-108, 24-108, 36-108, 48-108, 60-108, 72-108, 84-108, 96-108, 12-96, 24-96, 36-96, 48-96, 60-96, 72-96, 84-96, 12-84, 24-84, 36-84, 48-84, 60-84, 72-84, 12-72, 24-72, 36-72, 48-72, 60-72, 12-60, 24-60, 36-60, 48-60, 12-48, 24-48, 36-48, 42-48, 12-42, 18-42, 24-42, 30-42, 36-42, 12-36, 18-36, 24-36, or 12-24 hours.

In some embodiments, selective expansion is performed before or after physical cell sorting (e.g., via FACS or MACS). In some embodiments, physical cell sorting is not performed on cells after selective expansion.

In some embodiments, quality control assays are performed to verify gene editing, for example, knockin of a TCR of known specificity, disruption of a gene of interest, or a combination thereof. In some embodiments, quality control assays include, for example, flow cytometry, Western Blot, tracking of indels by decomposition (TIDE), polymerase chain reaction (PCR), nucleic acid sequencing, or a combination thereof.

In some embodiments, the percentage of cells in a population comprising a gene disruption or transgene insertion is quantified. In some embodiments, the percentage of cells in a population comprising a gene disruption or transgene insertion is quantified before or after enrichment as described herein. In some embodiments, a population of cells comprises at least about 5%, 10%, 15%, 20%, 25%, 30%, 35%, 40%, 45%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.5%, 99.9%, 99.99%, or more cells comprising the gene disruption or transgene insertion.

In some embodiments, before or after enrichment as described herein, a population of cells comprises at most about 5%, 10%, 15%, 20%, 25%, 30%, 35%, 40%, 45%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.5%, 99.9%, 99.99%, or more cells comprising the gene disruption or transgene insertion.

In some embodiments, before or after enrichment as described herein, a population of cells comprises between about 5% to 100%, 10% to 100%, 15% to 100%, 20% to 100%, 25% to 100%, 30% to 100%, 35% to 100%, 40% to 100%, 45% to 100%, 50% to 100%, 55% to 100%, 60% to 100%, 65% to 100%, 70% to 100%, 75% to 100%, 80% to 100, 85% to 100%, 90% to 100%, 95% to 100%, 96% to 100%, 97% to 100%, 98% to 100%, 99% to 100%, 99.5% to 100%, 5% to 95%, 10% to 95%, 15% to 95%, 20% to 95%, 25% to 95%, 30% to 95%, 35% to 95%, 40% to 95%, 45% to 95%, 50% to 95%, 55% to 95%, 60% to 95%, 65% to 95%, 70% to 95%, 75% to 95%, 80% to 95, 85% to 95%, 90% to 95%, 5% to 90%, 10% to 90%, 15% to 90%, 20% to 90%, 25% to 90%, 30% to 90%, 35% to 90%, 40% to 90%, 45% to 90%, 50% to 90%, 55% to 90%, 60% to 90%, 65% to 90%, 70% to 90%, 75% to 90%, 80% to 90, 85% to 90%, 5% to 85%, 10% to 85%, 15% to 85%, 20% to 85%, 25% to 85%, 30% to 85%, 35% to 85%, 40% to 85%, 45% to 85%, 50% to 85%, 55% to 85%, 60% to 85%, 65% to 85%, 70% to 85%, 75% to 85%, 80% to 85, 5% to 80%, 10% to 80%, 15% to 80%, 20% to 80%, 25% to 80%, 30% to 80%, 35% to 80%, 40% to 80%, 45% to 80%, 50% to 80%, 55% to 80%, 60% to 80%, 65% to 80%, 70% to 80%, 75% to 80%, 5% to 75%, 10% to 75%, 15% to 75%, 20% to 75%, 25% to 75%, 30% to 75%, 35% to 75%, 40% to 75%, 45% to 75%, 50% to 75%, 55% to 75%, 60% to 75%, 65% to 75%, 70% to 75%, 5% to 70%, 10% to 70%, 15% to 70%, 20% to 70%, 25% to 70%, 30% to 70%, 35% to 70%, 40% to 70%, 45% to 70%, 50% to 70%, 55% to 70%, 60% to 70%, 65% to 70%, 5% to 65%, 10% to 65%, 15% to 65%, 20% to 65%, 25% to 65%, 30% to 65%, 35% to 65%, 40% to 65%, 45% to 65%, 50% to 65%, 55% to 65%, 60% to 65%, 5% to 60%, 10% to 60%, 15% to 60%, 20% to 60%, 25% to 60%, 30% to 60%, 35% to 60%, 40% to 60%, 45% to 60%, 50% to 60%, 55% to 60%, 5% to 55%, 10% to 55%, 15% to 55%, 20% to 55%, 25% to 55%, 30% to 55%, 35% to 55%, 40% to 55%, 45% to 55%, 50% to 55%, 5% to 50%, 10% to 50%, 15% to 50%, 20% to 50%, 25% to 50%, 30% to 50%, 35% to 50%, 40% to 50%, 45% to 50%, 5% to 45%, 10% to 45%, 15% to 45%, 20% to 45%, 25% to 45%, 30% to 45%, 35% to 45%, 40% to 45%, 5% to 40%, 10% to 40%, 15% to 40%, 20% to 40%, 25% to 40%, 30% to 40%, 35% to 40%, 5% to 100%, 10% to 35%, 15% to 35%, 20% to 35%, 25% to 35%, 30% to 35%, 5% to 30%, 10% to 30%, 15% to 30%, 20% to 30%, 25% to 30%, 5% to 25%, 10% to 25%, 15% to 25%, 20% to 25%, 5% to 20%, 10% to 20%, 15% to 20%, 5% to 15%, 10% to 15%, or 5% to 10% cells comprising the gene disruption or transgene insertion.

In some embodiments, edited cells of the disclosure are used fresh or cryopreserved and later revived for use. In some embodiments, cells are cryopreserved using, for example, dimethyl sulfoxide (DMSO) as a cryoprotectant. In one embodiment, cells are cryopreserved in media comprising about 90% fetal bovine serum and about 10% DMSO.

### Co-Culture Assays

In order to evaluate the functional impact of disrupting a candidate immunomodulatory gene, T cells can be co-cultured with cells that express or present an antigen, for example, a cognate antigen recognized by a TCR of known specificity. In some embodiments, an antigen or cognate antigen is a neoantigen. In some embodiments, the response of T cells to cells that express or present the antigen (e.g., a cancer cell) is evaluated, for example, by quantifying cytotoxicity/cytolytic activity, cytokine production, proliferation, activation, maturation into memory or effector subsets, gene expression, protein expression, activation of signal transduction pathways, or any combination thereof. For example, in some embodiments, T cells are co-cultured with cancer cells expressing a particular antigen, and both cytolytic activity and another response can be measured. As described above, assaying for cytolytic activity can be particularly beneficial, and the addition of one or more other types of readout can enhance this beneficial effect. In some embodiments, the assessment of response is binary (e.g., cells expressing a cognate antigen of a T cell receptor or portion thereof are killed when co-cultured with cells having a disruption in the gene being tested, but are not killed when co-cultured with comparable cells that do not have that disruption). In some embodiments, the assessment of responses is graded (e.g., cells expressing a cognate antigen of a T cell receptor or portion thereof have lower survival/viability when co-cultured with cells having a disruption in the gene being tested, and higher survival/viability when co-cultured with comparable cells that do not have that disruption).

In some embodiments, T cells are co-cultured with cells that present an antigen via MHC-I, or a combination thereof. In some embodiments, antigen presenting cells are pulsed with an antigen, and co-cultured with T cells. In some embodiments, primary cells or cell lines are engineered to express an antigen or present the antigen via MHC-I, MHC-II, or a combination thereof. In some embodiments, primary cells or cancer cell lines known to express an antigen are used. In some embodiments, primary cells are primary cancer cells.

In some embodiments, T cells are co-cultured with cells that express or present G12D mutant KRAS. In some embodiments, T cells are co-cultured with cells that present G12D mutant KRAS via MHC-I, MHC-II, or a combination thereof. In some embodiments, antigen presenting cells are pulsed with G12D mutant KRAS, and co-cultured with T cells. In some embodiments, primary cells or cell lines are engineered to express G12D mutant KRAS or present G12D mutant KRAS via MHC-I, MHC-II or a combination thereof. In some embodiments, primary cells or cancer cell lines known to express G12D mutant KRAS are used.

In some embodiments, T cells are co-cultured with antigen presenting cells (APCs). Primary cells or cell lines can be APCs. In some embodiments, an APC expresses a cognate antigen for a T cell receptor and costimulatory molecules, and can activate T cells. In some embodiments, an APC can activate CD4 T cells. For example, an APC can be engineered to mimic an antigen processing and presentation pathway of MHC class II-restricted CD4 T cells. In some embodiments, an APC can activate CD8 T cells. In some embodiments, an APC can activate CD4 and CD8 T cells. An APC can be engineered to express HLA-D, DP α, DP β chains, Ii, DM α, DM β, CD80, CD83, or any combination thereof. For example, COS-7 cells can be engineered to express human MHC-I, and pulsed with G12D mutant KRAS.

An APC can be engineered to express any gene for T cell activation. An APC can deliver signals to a T cell. For example, an APC can deliver a signal 1, signal, 2, signal 3 or any combination thereof. A signal 1 can be an antigen recognition signal. For example, signal 1 can be ligation of a TCR by a peptide-MHC complex or binding of agonistic antibodies directed towards CD3 that can lead to activation of the CD3 signal-transduction complex. Signal 2 can be a co-stimulatory signal. For example, a co-stimulatory signal can bind to CD28, or to inducible co-stimulator (ICOS), CD27, or 4-1BB (CD137), which bind to ICOS-L, CD70, and 4-1BBL, respectively. Signal 3 can be a cytokine signal. A cytokine can be any cytokine. In some embodiments, said cytokine is IL-1α, IL-1β, IL-2, IL-4, IL-5, IL-6, IL-7, IL-9, IL-10, IL-12, IL-13, IL-15, IL-17, IL-21, IL-23, TNF-α, IFN-γ or any combination thereof.

Co-culture assays can be performed where the ratio of one cell type to another (e.g., T cells to target cells or T cells to APCs) is, for example, about 500:1, 400:1, 300:1, 250:1, 200:1, 150:1, 100:1, 90:1, 80:1, 70:1, 60:1, 50:1, 40:1, 30:1, 20:1, 10:1, 9:1, 8:1, 7:1, 6:1, 5:1, 4:1, 3:1, 2:1, 1:1, 1:2, 1:3, 1:4, 1:5, 1:6, 1:7, 1:8, 1:9, 1:10, 1:20, 1:30, 1:40, 1:50, 1:60, 1:70, 1:80, 1:90, 1:100, 1:150, 1:200, 1:250, 1:300, 1:400, or 1:500.

Co-culture assays can comprise incubation of two or more cell types, for example, for at least about 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 102, 108, 114, 120, 126, 132, 138, 144, 150, 156, 162, 168, 174, 180, 186, 192, 198, 204, 210, 216, 222, 228, 234, 240 hours, or more.

Co-culture assays can comprise incubation of two or more cell types, for example, for at most about 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 102, 108, 114, 120, 126, 132, 138, 144, 150, 156, 162, 168, 174, 180, 186, 192, 198, 204, 210, 216, 222, 228, 234, 240 hours, or less.

Co-culture assays can comprise incubation of two or more cell types, for example, for between about 2-240, 6-120, 12-96, 12-72, 12-48, 12-36, 12-24, 12-16, 16-96, 16-72, 16-48, 16-42, 16-36, 16-30, 16-24, 16-20, 16-18, 20-96, 20-72, 20-48, 20-42, 20-36, 20-30, 20-24, 24-96, 24-72, 24-48, 24-42, 24-36, 24-30, 28-96, 28-72, 28-48, 28-42, 28-36, 28-30, 32-96, 32-72, 32-48, 32-32, 32-36, 36-96, 36-72, 36-48, 36-42, 40-96, 40-72, 40-48, 40-42, 40-36, 40-30, 40-24, 40-18, 44-96, 44-72, 44-48, 48-96, 48-72, 52-96, 52-72, 56-96, 56-72, 60-96, or 60-72 hours.

Non-limiting examples of cancer cell lines known to express G12D mutant KRAS include AsPC-1 (pancreas-derived, ATCC), GP2d (large intestine-derived, Sigma), HPAF-II (pancreas-derived, ATCC), LS 180 (large intestine-derived, ATCC), LS513 (large intestine-derived, ATCC), Panc 02.03 (pancreas-derived, ATCC), Panc 04.03 (pancreas-derived, ATCC), Panc 08.13 (pancreas-derived, ATCC), Panc 10.05 (pancreas-derived, ATCC), PK-1 (Pancreas-derived, RIKEN), PK-45H (pancreas-derived, RIKEN), PK-59 (pancreas-derived, RIKEN), SK-LU-1 (lung-derived, ATCC), SNU-407 (large intestine-derived, Korean Cell Line Bank), SNU-C2A (large intestine-derived, ATCC), SU.86.86 (pancreas-derived, ATCC), SW 1990 (pancreas-derived, ATCC), and T3M-10 (lung-derived, RIKEN), or any genetically engineered isogenic cell line in which the KRAS G12D mutation, or other relevant genetic change, has been introduced via gene editing technologies.

In some embodiments, co-culture assays are performed in the presence of suppressive factors or conditions, for example, factors or conditions that reduce the T cell response to cognate antigen or CD3/CD28 co-stimulation. Non-limiting examples of suppressive factors or conditions include adenosine receptor agonists, suppressive cytokines (e.g. IL-10, TGF-β), suppressive cells (e.g. Tregs, MDSCs), cytostatics, alkylating agents, antimetabolites, glucocorticoids, methotrexate, tacrolimus, sirolimus, everolimus, ciclosporin, nutrient depletion, and combinations thereof.

In some embodiments, T cells are co-cultured with an acellular stimulus, such antibodies or beads that target CD2, CD3, CD28, or any combination thereof. In some embodiments, T cells are activated by peptide-MHC multimers, for example, tetramers or pentamers.

In some embodiments, one or more cell types in a co-culture assay are engineered to express one or more reporter genes, e.g., a fluorescent protein or luciferase.

### Functional Evaluation of T Cells

Provided herein are, inter alia, methods for identifying immunomodulatory genes, including, for example, disrupting candidate immunomodulatory genes, and then testing the effect of the disruptions on T cell function. Candidate immunomodulatory genes can be disrupted in T cells expressing a TCR of known specificity. In some embodiments, the T cells are then co-cultured with target cells, and a range of assays used to evaluate the effect of gene disruption on the T cell response upon recognition of cells presenting target antigen.

In some embodiments, cytotoxicity assays are used to evaluate the ability of T cells to kill cells expressing or presenting cognate antigen. In some embodiments, cytotoxicity assays are used that are based on the release of an intracellular enzyme by dead cells (e.g., lactate dehydrogenase, adenylate kinase, protease, or luciferase). In some embodiments, cytotoxicity assays are used that are based on the exclusion of dye by intact cell membranes, (e.g., SYTOX® Green nucleic acid stain, Image-iT® DEAD Green™ viability stain, 7-AAD, propidium iodide, amine-reactive dyes, trypan blue exclusion). In some embodiments, cytotoxicity assays are used wherein only metabolically active cells produce a signal (e.g., hydrolysis of Calcein AM to Calcein, reduction of MT cell viability substrate, conversion of resazurin to resorufin, conversion of a tetrazolium compound to formazan, live cell protease activity). In some embodiments, cytotoxicity assays are used wherein ATP is quantified. In some embodiments, cytotoxicity assays are used wherein time lapse microscopy is used, and cell confluence, reporter gene (e.g. GFP) expression, or caspase activation is measured. In some embodiments, cycotoxicity is determined by using a chromium release assay, wherein target cells are loaded with chromium, and chromium release upon cell killing measured with a gamma counter. In some embodiments, cytotoxicity assays comprise flow cytometric analysis of cells, with or without additional antibodies to identify other markers of interest. In some embodiments, target cells are modified or engineered to facilitate cytotoxicity measurement, e.g., engineered to express cytoplasmic luciferase, allowing for convenient detection luciferase release with appropriate reagents and a plate reader.

In some embodiments, the ability of T cells to produce cytokines in response to cells expressing or presenting cognate antigen are quantified. Non-limiting examples of methods for quantifying cytokine production include Enzyme-Linked Immunosorbent Assay (ELISA), multiplex immunoassay, intracellular cytokine staining, western blot, and quantitative real-time PCR. Non-limiting examples of cytokines that can be detected include IL-1α, IL-1β, IL-2, IL-4, IL-5, IL-6, IL-7, IL-9, IL-10, IL-12, IL-13, IL-15, IL-17, IL-21, IL-23, TNF-α, IFN-γ or any combination thereof.

In some embodiments, the ability of T cells to proliferate in response to cells expressing or presenting cognate antigen are quantified. In some embodiments, proliferation assays comprise quantification of DNA replication (e.g. BrdU incorporation assay, EdU incorporation assay). In some embodiments, proliferation assays comprise dye dilution as cells divide (e.g., CFSE, CytoPainter, or CellTrace dye dilution). In some embodiments, proliferation assays comprise flow cytometric analysis of cells, with or without additional antibodies to identify other markers of interest.

In some embodiments, T cell memory or activation markers are evaluated after co-culture with cells expressing or presenting cognate antigen. In some embodiments, T cell activation or memory marker assays comprise flow cytometric analysis of cells, with or without additional antibodies to identify other markers of interest. Non-limiting examples of T cell subsets that can be identified include naïve, effector memory (T_{EM}), central memory (T_{CM}), activated T cells, T_{H}1, T_{H}2, T_{H}9, T_{H}17, and Treg cells. Non-limiting examples of T cell markers that can be used in these assays include CCR4, CCR6, CCR7, CD3, CD4, CD8, CD25, CD27, CD28, CD45RA, CD45RO, CD57, CD62L, CD69, CD107a, CD122, CD 154, CD197, Crth2, CXCR3, CXCR5, p-ERK, p-p38, p-Stat1, p-Stat3, p-Stat5, p-Stat6, granzyme B, and XCL1.

### Genetic Modulation and Screening of Cancer Cells

In some embodiments, the assays described herein are used to screen a cancer cell to determine which genes, when modulated in said cancer cell, improve an immune cell's (e.g., a T cell's) capacity to recognize and/or kill the cancer cells. In some embodiments, said assay comprises utilizing a library of gRNAs that target genes in said cancer cell and/or the introduction of one or more transgenes. In some embodiments, the cancer cell is a primary cancer cell. In some embodiments, the cancer cell is a cancer cell line. The assay can be run in a similar manner to those described herein for the use of screening immune cells, whereas cancer cells, instead of immune cells, contain the genomic disruption and or transgene. In some embodiments, the gene disrupted encodes a protein that is a negative regulator of an immune response. In some embodiments, the gene encodes a checkpoint inhibitor ligand. In some embodiments, said checkpoint inhibitor ligand is a ligand for one of PD1, CTLA-4, TCRA, TRAC, adenosine A2a receptor (ADORA), CD276, V-set domain containing T cell activation inhibitor 1 (VTCN1), B and T lymphocyte associated (BTLA), indoleamine 2,3-dioxygenase 1 (IDO1), killer cell immunoglobulin-like receptor, three domains, long cytoplasmic tail, 1 (KIR3DL1), lymphocyte-activation gene 3 (LAG3), hepatitis A virus cellular receptor 2 (HAVCR2), V-domain immunoglobulin suppressor of T-cell activation (VISTA), natural killer cell receptor 2B4 (CD244), hypoxanthine phosphoribosyltransferase 1 (HPRT), adeno-associated virus integration site 1 (AAVS1), or chemokine (C—C motif) receptor 5 (gene/pseudogene) (CCR5), CD160 molecule (CD160), T-cell immunoreceptor with Ig and ITIM domains (TIGIT), CD96 molecule (CD96), cytotoxic and regulatory T-cell molecule (CRTAM), leukocyte associated immunoglobulin like receptor 1 (LAIR1), sialic acid binding Ig like lectin 7 (SIGLEC7), sialic acid binding Ig like lectin 9 (SIGLEC9), tumor necrosis factor receptor superfamily member 10b (TNFRSF10B), tumor necrosis factor receptor superfamily member 10a (TNFRSF10A), caspase 8 (CASP8), caspase 10 (CASP10), caspase 3 (CASP3), caspase 6 (CASP6), caspase 7 (CASP7), Fas associated via death domain (FADD), Fas cell surface death receptor (FAS), transforming growth factor beta receptor II (TGFBRII), transforming growth factor beta receptor I (TGFBR1), SMAD family member 2 (SMAD2), SMAD family member 3 (SMAD3), SMAD family member 4 (SMAD4), SKI proto-oncogene (SKI), SKI-like proto-oncogene (SKIL), TGFB induced factor homeobox 1 (TGIF1), programmed cell death 1 (PD-1), cytotoxic T-lymphocyte-associated protein 4 (CTLA4), interleukin 10 receptor subunit alpha (IL10RA), interleukin 10 receptor subunit beta (IL10RB), heme oxygenase 2 (HMOX2), interleukin 6 receptor (IL6R), interleukin 6 signal transducer (IL6ST), c-src tyrosine kinase (CSK), phosphoprotein membrane anchor with glycosphingolipid microdomains 1 (PAG1), signaling threshold regulating transmembrane adaptor 1 (SIT1), forkhead box P3 (FOXP3), PR domain 1 (PRDM1), basic leucine zipper transcription factor, ATF-like (BATF), guanylate cyclase 1, soluble, alpha 2 (GUCY1A2), guanylate cyclase 1, soluble, alpha 3 (GUCY1A3), guanylate cyclase 1, soluble, beta 2 (GUCY1B2), prolyl hydroxylase domain (PHD1, PHD2, PHD3) family of proteins, or guanylate cyclase 1, soluble, beta 3 (GUCY1B3), egl-9 family hypoxia-inducible factor 1 (EGLN1), egl-9 family hypoxia-inducible factor 2 (EGLN2), egl-9 family hypoxia-inducible factor 3 (EGLN3), protein phosphatase 1 regulatory subunit 12C (PPP1R12C), NAD-dependent deacetylase sirtuin 2 (SIRT2), or Protein Tyrosine Phosphatase Non-Receptor Type 1 (PTPN1).

In some embodiments, the assays described herein are configured to test combinations of targets in both immune cells (e.g., T cells) and target cancer cells simultaneously by combinatorial modulation of target genes in both the immune cells (e.g., T cells) and the cancer cells. Gene modulation of both cell populations can include knock in of a transgene, modulation of an endogenous gene, or knockout of an endogenous, or any combination thereof. This combinatorial assay can identify additive or synergistic targets whose modulation on both the immune cells (e.g., T cells) and cancer cells can enable the immune cells (e.g., T cells) to kill the cancer cells more quickly or with a more potent response, or both.

### Algorithms and Artificial Intelligence

Provided herein are, inter alia, methods for identifying immunomodulatory genes. In some embodiments, algorithms are used to aid the prediction, ranking, selection, or identification of candidate immunomodulatory genes, as illustrated by FIG. 3. For example, the results of an assay testing the effect of candidate immunomodulatory gene disruptions can be input into an algorithm, which can combine that data with other data, for example, prior assay results or database entries, and provide an output of ranked genes for follow-up experiments. In some embodiments, algorithms are used to rank candidate immunomodulatory genes based on screening assays and other weighted parameters, as illustrated by example 24 and FIG. 5A. In some embodiments, algorithms are used for iterative selection of candidate immunomodulatory genes to screen, as illustrated by example 25 and FIG. 5B. In some embodiments, algorithms are used to identify druggable immunomodulatory genes related to candidate genes that are poor drug targets, as illustrated by example 26 and FIG. 5C.

In some embodiments, algorithms and/or artificial intelligence are used to rank candidate immunomodulatory genes or to select candidate immunomodulatory genes for iterative rounds of screening. Non-limiting examples of possible algorithm workflows are provided in FIGS. 5 A-C. In some embodiments, an algorithm uses a scoring system to rank candidate immunomodulatory genes. Scores can be derived from, for example, an assay (e.g., an assay evaluating the cytotoxicity of T cells with a candidate immunomodulatory gene disrupted), scientific knowledge regarding a given gene, presence of functional domains, membership in a biological pathway, membership in a signaling pathway, membership in a protein superfamily/family/subfamily, designation as ‘druggable’ or part of the ‘druggable genome’, subcellular localization, expression in T cells, expression in a tissue of interest, availability of crystal structure data, designation as a receptor, clinical trial history, designation as a target of an existing drug, designation as a target of a previous drug development candidate, designation as a target of a drug currently under development, association with known diseases, loss of function association with human disease, loss of function phenotype in mice, amenability to targeting by CRISPR/gRNAs, or any combination thereof.

In some embodiments, weighting factors are applied so that some scoring parameters contribute more to the final score and ranking than other scoring parameters. For example, a score from a cytotoxicity assay can be weighted to contribute more to the final score than a score derived from a loss of function phenotype in mice.

In some embodiments, a scoring system includes scores derived from an assay. In some embodiments, a scoring system does not include scores derived from an assay. In some embodiments, results from an assay are input into an algorithm, which converts the results to scores, adds additional scores derived from other sources, weights the scores, and ranks genes according to the combination of weighted scores. In some embodiments, a ranked list of genes is used to determine genes targeted in a subsequent experiment or analysis.

In some embodiments, the number of genes targeted in a subsequent experiment or analysis is greater than at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 35, 105, 110, 115, 120, 125, 130, 135, 140, 145, 150, 160, 170, 180, 190, 192, 200, 250, 288, 300, 384, 400, 500, 600, 700, 800, 900, 350, 2500, 3000, 4000, 5000, 6000, 7000, 8000, 9000, 3500, 15000, 20000, 25000, or 30000 genes. In some embodiments, the number of genes targeted in a subsequent experiment or analysis is at most about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 35, 105, 110, 115, 120, 125, 130, 135, 140, 145, 150, 160, 170, 180, 190, 192, 200, 250, 288, 300, 384, 400, 500, 600, 700, 800, 900, 350, 2500, 3000, 4000, 5000, 6000, 7000, 8000, 9000, 3500, 15000, 20000, 25000, or 30000 genes.

In some embodiments, scores are derived from gene characteristics extracted from databases. Non-limiting examples of databases include AmiGO, BiND, BioCarta, BioGPS, CAZy, CDD, COG, COMPARTMENTS, CTD, DAVID, DGIdb, DisGeNet, drugbank, eDGAR, EndoNet, Ensembl, Entrez, ExPASy, Expression Atlas, GAD, Gene Expression Omnibus, Gene Ontology, GeneWiki, GoGene, GXD, HAPMAP, HMGD, HOGENOM, HSLS, HUGO, HumanCyc, ImmunoDB, iPathwayGuide, the Kyoto Encyclopedia of Genes and Genomes (KEGG), KEGG PATHWAY, KEGG BRITE, KEGG MODULE, KEGG ORTHOLOGY, KEGG GENOME, KEGG GENES, KEGG COMPOUND, KEGG GLYCAN, KEGG REACTION, KEGG ENZYME, KEGG NETWORK, KEGG DISEASE, KEGG DRUG, KOG, the Human Protein Atlas, LHDGN, LocDB, LOCATE, MalaCards, MetaCyc, METAGENE, MGD, MGI, MouseMine, NCBI, NCBI-gene, NCBI-protein, NCBI-structure, NetDecoder, OMIM, OMMBID, OrthoDB, PANTHER, PathJam, Pathguide, Pathway Commons, Pfam, photon, Phyre2, PSORTdb, PID, PRK, ProDom, PROFESS, PROSITE, reactome, RefSeq, SIFT, SMART, SMPDB, SPATIAL, STRING, SuperTarget, Swiss-MODEL, Swiss-Prot, TIGR, Treefam, TTD, and UniProt.

In some embodiments, an algorithm comprises one or more of a machine learning algorithm, Hidden Markov Model, a dynamic programming algorithm, a support vector machine, a Bayesian network, a naive Bayesian algorithm, a trellis decoding algorithm, a Viterbi decoding algorithm, an expectation maximization algorithm, a Kalman filtering methodology, a neural network algorithm, a k-nearest neighbor algorithm, a concept vector algorithm, a genetic algorithm, a mutual information feature selection algorithm, a principal component analysis algorithm, a partial least squares algorithm, an independent component analysis algorithm, or any combination thereof.

Illustrative algorithms further include, but are not limited, to methods that handle large numbers of variables directly such as statistical methods, and methods based on machine learning techniques. Statistical methods include penalized logistic regression, methods based on shrunken centroids, support vector machine analysis, correlation analysis, and regularized linear discriminant analysis. Machine learning techniques include bagging procedures, boosting procedures, random forest algorithms, and combinations thereof.

In some embodiments, a machine learning algorithm is used. In some embodiments, a machine learning algorithm comprises a training step, for example, using a reference set of known immunomodulatory genes, or results from an earlier round of screening in which candidate immunomodulatory genes were disrupted and the resulting T cells functionally evaluated. In some embodiments, a machine learning algorithm is supervised or unsupervised.

In some embodiments, an algorithm comprises a Hidden Markov model (HMM), which is a statistical Markov model in which the system being modeled is assumed to be a Markov process with unobserved (hidden) states. In a simple Markov models (like a Markov chain), the state is directly visible to the observer, and therefore the state transition probabilities are the only parameters. In a hidden Markov model, the state is not directly visible, but output, dependent on the state, is visible. Each state has a probability distribution over the possible output tokens. Therefore, the sequence of tokens generated by an HMM may give some information about the sequence of states. A hidden Markov model can be considered a generalization of a mixture model where the hidden variables (or latent variables), which control the mixture component to be selected for each observation, are related through a Markov process rather than independent of each other. An HMM is typically defined by a set of hidden states, a matrix of state transition probabilities, and a matrix of emission probabilities. General methods to construct such models include, but are not limited to, Hidden Markov Models (HMM), artificial neural networks, Bayesian networks, support vector machines, and Random Forest. Such methods are known to one of ordinary skill in the art and are described in detail in Mohri et al., Foundations of Machine Learning, published by MIT Press (2012), which is hereby incorporated by reference in its entirety.

In some embodiments, an algorithm ranks genes using a Bayesian post-analysis method. For example, data can be subjected to a feature selection step. In some embodiments, the data is then subjected to a classification step comprising any of the algorithms or methods provided herein, for example, a support vector machine or Random Forest algorithm. In some embodiments, the results of the classifier algorithm are then ranked by according to a posterior probability function. For example, the posterior probability function can be derived from examining known immunomodulatory genes, to derive prior probabilities. These prior probabilities can then be combined with a dataset provided by the methods disclosed herein to estimate a posterior probability. The posterior probability estimates can be combined with a second dataset provided by the methods disclosed herein to formulate additional posterior probabilities. In some embodiments, posterior probabilities are used to rank genes provided by the classifier algorithm. In some embodiments, genes are ranked according to their posterior probabilities and those that pass a chosen threshold may be chosen. Illustrative threshold values include, but are not limited to, probabilities of 0.7, 0.75, 0.8, 0.85, 0.9, 0.925, 0.95, 0.975, 0.98, 0.985, 0.99, 0.995 or higher.

### Gene Editing Techniques

In some embodiments, cells of the disclosure are genetically edited, for example, to generate populations of primary T cells that can be screened to identify novel immunomodulatory genes.

In some embodiments, cells of the disclosure are genetically edited, for example, to generate primary T cells expressing a T cell receptor (TCR) of known specificity, to disrupt expression of an endogenous TCR, to disrupt expression of a known immunomodulatory gene, to disrupt expression of a candidate immunomodulatory gene, to generate cells that will activate T cells expressing a TCR of a known specificity, to generate cells comprising a polynucleotide of interest, to generate cells comprising a disrupted polynucleotide of interest, to disrupt expression of a gene of interest, or any combination thereof. In some embodiments, cells are genetically edited to generate cells comprising a TCR of known specificity, disrupted expression of an endogenous TCR, and disrupted expression of a candidate immunomodulatory gene. In some embodiments, cells are genetically edited to generate cells comprising a TCR of known specificity, disrupted expression of an endogenous TCR, and disrupted expression of a known immunomodulatory gene, e.g. PD-1. In some embodiments, cells are genetically edited to generate cells comprising a TCR of known specificity, disrupted expression of an endogenous TCR, disrupted expression of a candidate immunomodulatory gene, and disrupted expression of a known immune checkpoint gene, e.g. PD-1.

### Polynucleic Acids and Polynucleic Acid Modifications

In some embodiments, methods disclosed herein comprise introducing into a cell one or more nucleic acids. In some embodiments, nucleic acids are introduced into a cell, for example, as part of a process to genetically edit a T cell to disrupt an endogenous TCR, introduce a gene encoding a TCR of known specificity, disrupt a candidate immunomodulatory gene, disrupt a known immunomodulatory gene, or any combination thereof.

In some embodiments, a nucleic acid is a polynucleic acid. A person of skill in the art will appreciate that a nucleic acid may generally refer to a substance whose molecules consist of many nucleotides linked in a long chain. Non-limiting examples of polynucleic acids include, but are not limited to, an artificial nucleic acid analog (e.g., a peptide nucleic acid, a morpholino oligomer, a locked nucleic acid, a glycol nucleic acid, or a threose nucleic acid), a circular nucleic acid, a DNA, a single stranded DNA, a double stranded DNA, a genomic DNA, a plasmid, a plasmid DNA, a viral DNA, a viral vector, a gamma-retroviral vector, a lentiviral vector, an adeno-associated viral vector, an RNA, short hairpin RNA, psiRNA and/or a hybrid or combination thereof. In some embodiments, the polynucleic acid is synthetic. In some embodiments, a sample comprises a polynucleic acid, and the polynucleic acid is fragmented. In some embodiments, a polynucleic acid is a minicircle.

In some embodiments, the polynucleic acids as described herein are modified. A modification can be made at any location of a polynucleic acid. More than one modification can be made to a single polynucleic acid. A polynucleic acid can undergo quality control after a modification. In some cases, quality control may include PAGE, HPLC, MS, or any combination thereof.

In some cases, a polynucleic acid is modified to make it less immunogenic and more stable for transfection into a cell. In some embodiments, a modified polynucleic acid encodes any number of genes. In some cases, a polynucleic acid encodes a transgene. In some embodiments, a transgene encodes an engineered receptor. In some embodiments, a receptor is a T cell receptor (TCR), B cell receptor (BCR), chimeric antigen receptor (CAR), or any combination thereof. In some cases, a receptor is a TCR.

In some cases, a modified polynucleic acid is used in subsequent steps of processes described herein. For example, in some embodiments, a modified polynucleic acid is used in a homologous recombination reaction. In some embodiments, a homologous recombination reaction includes introducing a transgene encoding an exogenous receptor in a genome of a cell. In some embodiments, an introduction includes any mechanism necessary to introduce a transgene sequence into a genome of a cell. In some embodiments, CRISPR is used in steps to introduce a receptor sequence into a genome of a cell.

In some embodiments, a modification is permanent. In some embodiments, a modification is transient. In some embodiments, multiple modifications are made to a polynucleic acid. In some embodiments, a polynucleic acid modification alters physio-chemical properties of the polynucleic acid, such as its conformation, polarity, hydrophobicity, chemical reactivity, base-pairing interactions, or any combination thereof.

In some embodiments, a modification is a substitution, insertion, deletion, chemical modification, physical modification, stabilization, purification, or any combination thereof. In some embodiments, a polynucleic acid is modified by 5′adenylate, 5′ guanosine-triphosphate cap, 5′N⁷-Methylguanosine-triphosphate cap, 5′triphosphate cap, 3′phosphate, 3′thiophosphate, 5′phosphate, 5′thiophosphate, Cis-Syn thymidine dimer, trimers, C12 spacer, C3 spacer, C6 spacer, dSpacer, PC spacer, rSpacer, Spacer 18, Spacer 9,3′-3′ modifications, 5′-5′ modifications, abasic, acridine, azobenzene, biotin, biotin BB, biotin TEG, cholesteryl TEG, desthiobiotin TEG, DNP TEG, DNP-X, DOTA, dT-Biotin, dual biotin, PC biotin, psoralen C2, psoralen C6, TINA, 3′DABCYL, black hole quencher 1, black hole quencher 2, DABCYL SE, dT-DABCYL, IRDye QC-1, QSY-21, QSY-35, QSY-7, QSY-9, carboxyl linker, thiol linkers, 2′ deoxyribonucleoside analog purine, 2′ deoxyribonucleoside analog pyrimidine, ribonucleoside analog, 2′-O-methyl ribonucleoside analog, sugar modified analogs, wobble/universal bases, fluorescent dye label, 2′fluoro RNA, 2′O-methyl RNA, methylphosphonate, phosphodiester DNA, phosphodiester RNA, phosphothioate DNA, phosphorothioate RNA, UNA, pseudouridine-5′-triphosphate, 5-methylcytidine-5′-triphosphate, or any combination thereof.

In some embodiments, a modification is a 2-O-methyl 3 phosphorothioate addition. In some embodiments, a 2-O-methyl 3 phosphorothioate addition is added on from 1 base to 150 bases. In some embodiments, a 2-O-methyl 3 phosphorothioate addition is added on from 1 base to 4 bases. In some embodiments, a 2-O-methyl 3 phosphorothioate addition is added on 2 bases. In some embodiments, a 2-O-methyl 3 phosphorothioate addition is added on 4 bases. In some embodiments, a modification is a truncation. In some embodiments, a truncation is a 5 base truncation.

In some embodiments, a modification is be a phosphorothioate substitute. In some embodiments, a natural phosphodiester bond is susceptible to rapid degradation by cellular nucleases and a modification of internucleotide linkage using phosphorothioate (PS) bond substitutes enhances stability. In some embodiments, a modification increases stability in a polynucleic acid. In some embodiments, a modification enhances biological activity. In some embodiments, a phosphorothioate enhanced RNA polynucleic acid inhibits RNase A, RNase T1, calf serum nucleases, or any combinations thereof. In some embodiments, these properties allow PS-RNA polynucleic acids to be used in applications where there is high probability of exposure to nucleases in vivo or in vitro. In some embodiments, for example, phosphorothioate (PS) bonds are introduced between the last 3-5 nucleotides at the 5′- or 3′-end of a polynucleic acid which inhibit exonuclease degradation. In some embodiments, phosphorothioate bonds are added throughout an entire polynucleic acid to reduce attack by endonucleases.

In some embodiments, polynucleic acids are assembled by a variety of methods, e.g., by automated solid-phase synthesis. In some embodiments, a polynucleic acid is constructed using standard solid-phase DNA/RNA synthesis. In some embodiments, a polynucleic acid is constructed using a synthetic procedure. In some embodiments, a polynucleic acid is synthesized either manually or in a fully automated fashion. In some embodiments, a synthetic procedure is used wherein 5′-hydroxyl oligonucleotides can be initially transformed into corresponding 5′-H-phosphonate mono esters, subsequently oxidized in the presence of imidazole to activated 5′-phosphorimidazolidates, and finally reacted with pyrophosphate on a solid support. In some embodiments, this procedure includes a purification step after the synthesis such as PAGE, HPLC, MS, or any combination thereof

### Ribonucleic Acid System

One exemplary method of generating genetically edited cells is through the use of a ribonucleic acid (RNA) system, e.g., a full or partial RNA system for intracellular genomic transplant. In some embodiments, cells to be engineered are genetically modified with RNA or modified RNA instead of DNA to prevent DNA (e.g., double or single stranded DNA)-induced toxicity and immunogenicity sometimes observed with the use of DNA. In some embodiments, an RNA/DNA fusion polynucleic acid is employed for genomic engineering.

In some embodiments, an all RNA polynucleic acid system for gene editing of primary human T cells is used. In some embodiments, an in vitro transcribed ribonucleic acid is delivered and reverse transcribed into dsDNA inside a target cell. In some embodiments, a DNA template is used for a homologous recombination (HR) reaction inside the cell.

In some embodiments, a transgene comprising an exogenous receptor sequence is introduced into a cell for genome engineering via RNA, e.g., messenger RNA (mRNA). RNA, e.g., mRNA can be converted to DNA in situ. One exemplary method utilizes in vitro transcription of a polynucleic acid to produce an mRNA polynucleic acid. In some embodiments, an mRNA polynucleic acid is then transfected into a cell with a reverse transcriptase (RT) (either in protein form or a polynucleic acid encoding for a RT). In some embodiments, an RT is derived from Avian Myeloblastosis Virus Reverse Transcriptase (AMV RT), Moloney murine leukemia virus reverse transcriptase (M-MLV RT), human immunodeficiency virus (HIV) reverse transcriptase (RT), derivatives thereof or combinations thereof. In some embodiments, once transfected, a reverse transcriptase transcribes the engineered mRNA polynucleic acid into a double stranded DNA (dsDNA). In some embodiments, a reverse transcriptase (RT) is an enzyme used to generate complementary DNA (cDNA) from an RNA template. In some embodiments, a double stranded DNA is used in a subsequent homologous recombination step. In some embodiments, a subsequent homologous recombination step introduces an exogenous receptor sequence into the genome of a cell.

In some embodiments, an introduced RT is targeted to an introduced polynucleic acid. In some embodiments, an introduced polynucleic acid is a RNA or DNA. In some embodiments, an introduced polynucleic acid is a combination of RNA and DNA. In some embodiments, targeting an introduced RT is performed by incorporating a unique sequence into a polynucleic acid encoding for an engineered receptor. In some embodiments, these unique sequences help target the RT to a particular polynucleic acid. In some embodiments, a unique sequence can increase efficiency of a reaction.

Table 2 describes possible unique sequences to target an RT to an engineered polynucleic acid.

TABLE 2
Unique Sequences

| SEQ ID NO: | Unique Sequence 5′ to 3′ |
|---|---|
| 1 | TAGTCGGTACGCGACTAAGCCG |
| 2 | TAGTCGTCGTAACGTACGTCGG |
| 3 | CGGCTATAACGCGTCGCGTAG |
| 4 | TAGAGCGTACGCGACTAACGAC |

In some embodiments, a reverse transcriptase is targeted to an engineered polynucleic acid by engineering the polynucleic acid to have a secondary structure. In some embodiments, a secondary structure is any structure. In some embodiments, multiple secondary structures are utilized. For example, in some embodiments, a secondary structure is a double helix. In some embodiments, a secondary structure is a stem-loop or hairpin structure. In some embodiments, a secondary structure is a pseudoknot.

In some embodiments, an engineered polynucleic acid needs to be localized to a cellular nucleus. In some embodiments, an engineered polynucleic acid encodes an exogenous or engineered receptor sequence that needs to be introduced into a genome of a cell. In some embodiments, introducing a receptor sequence to a cell genome is performed by localizing an engineered polynucleic acid to a cell nuclease for transcription.

In some embodiments, an engineered RNA polynucleic acid is localized to a cellular nucleus. In some embodiments, localization comprises any number of techniques. In some embodiments, a nuclear localization signal is used to localize an engineered polynucleic acid encoding an engineered receptor to a nucleus. In some embodiments, a nuclear localization signal is any endogenous or engineered sequence.

### CRISPR System

In some embodiments, methods described herein use a CRISPR system, for example, to generate a double stranded break in a target gene in order to knock out a candidate immunomodulatory gene, knock out a known immunomodulatory gene, knock out an endogenous TCR, knockin a TCR of known specificity, or any combination thereof.

There are at least five types of CRISPR systems which all incorporate RNAs and CRISPR-associated (Cas) proteins. Types I, III, and IV assemble a multi-Cas protein complex that is capable of cleaving nucleic acids that are complementary to the crRNA. Types I and III both require pre-crRNA processing prior to assembling the processed crRNA into the multi-Cas protein complex. Types II and V CRISPR systems comprise a single Cas protein complexed with at least one guide RNA (gRNA).

The general mechanism and recent advances of CRISPR system is discussed in Cong, L. et al., “Multiplex genome engineering using CRISPR systems,” Science, 339(6121): 819-823 (2013); Fu, Y. et al., “High-frequency off-target mutagenesis induced by CRISPR-Cas nucleases in human cells,” Nature Biotechnology, 31, 822-826 (2013); Chu, V T et al. “Increasing the efficiency of homology-directed repair for CRISPR-Cas9-induced precise gene editing in mammalian cells,” Nature Biotechnology 33, 543-548 (2015); Shmakov, S. et al., “Discovery and functional characterization of diverse Class 2 CRISPR-Cas systems,” Molecular Cell, 60, 1-13 (2015); Makarova, K S et al., “An updated evolutionary classification of CRISPR-Cas systems,”, Nature Reviews Microbiology, 13, 1-15 (2015).

Site-specific cleavage of a target DNA occurs at locations determined by both 1) base-pairing complementarity between the guide RNA and the target DNA (also called a protospacer) and 2) a short motif in the target DNA referred to as the protospacer adjacent motif (PAM). For example, an engineered cell can be generated using a CRISPR system, e.g., a type II CRISPR system. A Cas enzyme used in the methods disclosed herein can be Cas9, which catalyzes DNA cleavage. Enzymatic action by Cas9 derived from Streptococcus pyogenes or any closely related Cas9 can generate double stranded breaks at target site sequences which hybridize to 20 nucleotides of a guide sequence and that have a protospacer-adjacent motif (PAM) following the 20 nucleotides of the target sequence.

a. Cas Protein

In some embodiments, a CRISPR-associated (Cas) protein comprises an enzymatic activity to generate a double-stranded break (DSB) in DNA, at a site determined by a guide RNA (gRNA).

In some embodiments, a method can comprise an endonuclease selected from the group consisting of Cas1, Cas1B, Cas2, Cas3, Cas4, Cas5, Cas6, Cas7, Cas8, Cas9, Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx1S, Csf1, Csf2, CsO, Csf4, Cpf1, c2c1, c2c3, Cas9HiFi, homologues thereof or modified versions thereof. A Cas protein can be Cas9.

In some embodiments, a vector is operably linked to an enzyme-coding sequence encoding a CRISPR enzyme, such as a Cas protein (CRISPR-associated protein). Non-limiting examples of Cas proteins include, but are not limited to, Cas1, Cas1B, Cas2, Cas3, Cas4, Cas5, Cas6, Cas7, Cas8, Cas9 (also known as Csn1 or Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx1S, Csf1, Csf2, CsO, Csf4, Cpf1, c2c1, c2c3, Cas9HiFi, homologues thereof, or modified versions thereof. In some embodiments, an unmodified CRISPR enzyme has DNA cleavage activity, such as Cas9.

In some embodiments, a CRISPR enzyme directs cleavage of one or both strands at a target sequence, such as within a target sequence and/or within a complement of a target sequence. For example, In some embodiments, a CRISPR enzyme directs cleavage of one or both strands within or within about 1, 2, 3, 4, 5, 6, 7, 8, 9, 10, 15, 20, 25, 50, 35, 200, 500, or more base pairs from the first or last nucleotide of a target sequence. In some embodiments, a vector that encodes a CRISPR enzyme that is mutated with respect to a corresponding wild-type enzyme such that the mutated CRISPR enzyme lacks the ability to cleave one or both strands of a target polynucleotide containing a target sequence is used. In some embodiments, a Cas protein is a high fidelity Cas protein such as Cas9HiFi.

In some embodiments, a vector that encodes a CRISPR enzyme comprising one or more nuclear localization sequences (NLSs), such as more than or more than about 1, 2, 3, 4, 5, 6, 7, 8, 9, 10, NLSs is used. For example, In some embodiments, a CRISPR enzyme comprise more than or more than about 1, 2, 3, 4, 5, 6, 7, 8, 9, 10, NLSs at or near the ammo-terminus, more than or more than about 1, 2, 3, 4, 5, 6, 7, 8, 9, 10, NLSs at or near the carboxyl-terminus, or any combination of these (e.g., one or more NLS at the N-terminus and one or more NLS at the C-terminus). The NLS can be located anywhere within the polypeptide chain, e.g., near the N- or C-terminus. For example, In some embodiments, the NLS is within or within about 1, 2, 3, 4, 5, 10, 15, 20, 25, 30, 40, 50 amino acids along a polypeptide chain from the N- or C-terminus. In some embodiments, the NLS is within or within about 50 amino acids or more, e.g., 35, 200, 300, 400, 500, 600, 700, 800, 900, or 350 amino acids from the N- or C-terminus. In some embodiments, when more than one NLS is present, each is selected independently of others, such that a single NLS can be present in more than one copy and/or in combination with one or more other NLSs present in one or more copies.

In some embodiments, cas9 refers to a polypeptide with at least or at least about 50%, 60%, 70%, 80%, 90%, 35% sequence identity and/or sequence similarity to a wild type exemplary Cas9 polypeptide (e.g., Cas9 from S. pyogenes). In some embodiments, cas9 refers to a polypeptide with at most or at most about 50%, 60%, 70%, 80%, 90%, 35% sequence identity and/or sequence similarity to a wild type exemplary Cas9 polypeptide (e.g., from S. pyogenes). In some embodiments, cas9 refers to the wild type or a modified form of the Cas9 protein that comprises an amino acid change such as a deletion, insertion, substitution, variant, mutation, fusion, chimera, or any combination thereof.

In some embodiments, a polynucleotide encoding an endonuclease (e.g., a Cas protein such as Cas9) is codon optimized for expression in particular cells, such as eukaryotic cells. In some embodiments, this type of optimization entails the mutation of foreign-derived (e.g., recombinant) DNA to mimic the codon preferences of the intended host organism or cell while encoding the same protein.

In some embodiments, an endonuclease comprises an amino acid sequence having at least or at least about 50%, 60%, 70%, 75%, 80%, 85%, 90%, 95%, 99%, or 35%, amino acid sequence identity to the nuclease domain of a wild type exemplary site-directed polypeptide (e.g., Cas9 from S. pyogenes).

While S. pyogenes Cas9 (SpCas9), can be used as a CRISPR endonuclease for genome engineering, other endonucleases may also be useful for certain target excision sites. For example, the PAM sequence for SpCas9 (5′ NGG 3′) is abundant throughout the human genome, but an NGG sequence may not be positioned correctly to target a desired gene for modification. In some embodiments, a different endonuclease is used to target certain genomic targets. In some embodiments, synthetic SpCas9-derived variants with non-NGG PAM sequences is used. Additionally, other Cas9 orthologues from various species have been identified and these “non-SpCas9s” bind a variety of PAM sequences that could also be useful for the present disclosure. For example, the relatively large size of SpCas9 (approximately 4 kb coding sequence) means that plasmids carrying the SpCas9 cDNA may not be efficiently expressed in a cell. Conversely, the coding sequence for Staphylococcus aureus Cas9 (SaCas9) is approximately 1 kilo base shorter than SpCas9, possibly allowing it to be efficiently expressed in a cell. Similar to SpCas9, the SaCas9 endonuclease is capable of modifying target genes in mammalian cells in vitro and in mice in vivo.

Alternatives to S. pyogenes Cas9 include, but are not limited to, RNA-guided endonucleases from the Cpf1 family that display cleavage activity in mammalian cells. Unlike Cas9 nucleases, the result of Cpf1-mediated DNA cleavage is a double-strand break with a short 3′ overhang. Cpf1's staggered cleavage pattern may open up the possibility of directional gene transfer, analogous to traditional restriction enzyme cloning, which may increase the efficiency of gene editing. Like the Cas9 variants and orthologues described above, Cpf1 may also expand the number of sites that can be targeted by CRISPR to AT-rich regions or AT-rich genomes that lack the NGG PAM sites favored by SpCas9.

Any functional concentration of Cas protein can be introduced to a cell. For example, In some embodiments, 15 micrograms of Cas mRNA is introduced to a cell. In some embodiments, a Cas mRNA is introduced from 0.5 micrograms to 35 micrograms. In some embodiments, a Cas mRNA of about 0.5, 5, 10, 15, 20, 25, 30, 35, 40, 45, 50, 55, 60, 65, 70, 75, 80, 85, 90, 95, or 35 micrograms is introduced.

b. Guide RNA

As used herein, the term “guide RNA (gRNA)”, and its grammatical equivalents refer to a RNA which can be specific for a target DNA and can form a complex with a Cas protein. In some embodiments, a guide RNA comprises a guide sequence, or spacer sequence, that specifies a target site and guides a RNA/Cas complex to a specified target DNA for cleavage. Site-specific cleavage of a target DNA occurs at locations determined by both 1) base-pairing complementarity between a guide RNA and a target DNA (also called a protospacer) and 2) a short motif in a target DNA referred to as a protospacer adjacent motif (PAM).

In some embodiments, methods disclosed herein comprise introducing into a cell at least one guide RNA or nucleic acid, e.g., DNA encoding at least one guide RNA. In some embodiments, a guide RNA interacts with an RNA-guided endonuclease to direct the endonuclease to a specific target site, at which site the 5′ end of the guide RNA base pairs with a specific protospacer sequence in a chromosomal sequence.

In some embodiments, a guide RNA comprises two RNAs, e.g., CRISPR RNA (crRNA) and transactivating crRNA (tracrRNA). In some embodiments, a guide RNA comprises a single-guide RNA (sgRNA) formed by fusion of a portion (e.g., a functional portion) of crRNA and tracrRNA. In some embodiments, a guide RNA is a dual RNA comprising a crRNA and a tracrRNA. In some embodiments, a guide RNA comprises a crRNA and lack a tracrRNA. Furthermore, In some embodiments, a crRNA hybridizes with a target DNA or protospacer sequence.

As discussed above, In some embodiments, a gRNA is an expression product. For example, In some embodiments, a DNA that encodes a gRNA is a vector comprising a sequence coding for the gRNA. In some embodiments, a gRNA is transferred into a cell or organism by transfecting the cell or organism with an isolated gRNA or plasmid DNA comprising a sequence coding for the gRNA and a promoter. In some embodiments, a gRNA is transferred into a cell or organism by transduction with a viral vector. In some embodiments, gRNAs are delivered by a retrovirus, such as a lentiviral vector. In some embodiments, a gRNAs are delivered by an adenovirus, parvovirus (e.g., adeno-associated virus (AAV)), retrovirus, herpesvirus, or integrase-defective lentivirus (IDLV). In some embodiments, gRNAs can be delivered via electroporation.

In some embodiments, a guide RNA is isolated. For example, In some embodiments, a guide RNA is transfected in the form of an isolated RNA into a cell or organism. In some embodiments, a guide RNA is prepared by in vitro transcription using any in vitro transcription system. In some embodiments, a guide RNA can be transferred to a cell in the form of isolated RNA rather than in the form of plasmid comprising encoding sequence for a guide RNA.

In some embodiments, a guide RNA comprises a DNA-targeting segment and a protein binding segment. In some embodiments, a DNA-targeting segment (or DNA-targeting sequence, or spacer sequence) comprises a nucleotide sequence that can be complementary to a specific sequence within a target DNA (e.g., a protospacer). In some embodiments, a protein-binding segment (or protein-binding sequence) interacts with a site-directed modifying polypeptide, e.g. an RNA-guided endonuclease such as a Cas protein. In some embodiments, a segment is a segment/section/region of a molecule, e.g., a contiguous stretch of nucleotides in an RNA. In some embodiments, a segment is mean a region/section of a complex such that a segment may comprise regions of more than one molecule. For example, In some embodiments, a protein-binding segment of a DNA-targeting RNA is one RNA molecule and the protein-binding segment therefore comprises a region of that RNA molecule. In some embodiments, the protein-binding segment of a DNA-targeting RNA comprises two separate molecules that are hybridized along a region of complementarity.

In some embodiments, a guide RNA comprises two separate RNA molecules or a single RNA molecule. An exemplary single molecule guide RNA comprises both a DNA-targeting segment and a protein-binding segment.

An exemplary two-molecule DNA-targeting RNA comprises a crRNA-like (“CRISPR RNA” or “targeter-RNA” or “crRNA” or “crRNA repeat”) molecule and a corresponding tracrRNA-like (“trans-acting CRISPR RNA” or “activator-RNA” or “tracrRNA”) molecule. A first RNA molecule can be a crRNA-like molecule (targeter-RNA), that can comprise a DNA-targeting segment (e.g., spacer) and a stretch of nucleotides that can form one half of a double-stranded RNA (dsRNA) duplex comprising the protein-binding segment of a guide RNA. In some embodiments, a second RNA molecule is a corresponding tracrRNA-like molecule (activator-RNA) that comprises a stretch of nucleotides that can form the other half of a dsRNA duplex of a protein-binding segment of a guide RNA. In other words, In some embodiments, a stretch of nucleotides of a crRNA-like molecule is complementary to and hybridizes with a stretch of nucleotides of a tracrRNA-like molecule to form a dsRNA duplex of a protein-binding domain of a guide RNA. As such, In some embodiments, each crRNA-like molecule has a corresponding tracrRNA-like molecule. In some embodiments, a crRNA-like molecule additionally provides a single stranded DNA-targeting segment, or spacer sequence. Thus, In some embodiments, a crRNA-like and a tracrRNA-like molecule (as a corresponding pair) hybridizes to form a guide RNA. In some embodiments, a subject two-molecule guide RNA comprises any corresponding crRNA and tracrRNA pair.

In some embodiments, a DNA-targeting segment or spacer sequence of a guide RNA is complementary to sequence at a target site in a chromosomal sequence, e.g., protospacer sequence) such that the DNA-targeting segment of the guide RNA can base pair with the target site or protospacer. In some embodiments, a DNA-targeting segment of a guide RNA comprises from or from about 10 nucleotides to from or from about 25 nucleotides or more. For example, In some embodiments, a region of base pairing between a first region of a guide RNA and a target site in a chromosomal sequence is about 10, 11, 12, 13, 14, 15, 16, 17, 18, 19, 20, 22, 23, 24, 25, or more than 25 nucleotides in length. In some embodiments, a first region of a guide RNA is about 19, 20, or 21 nucleotides in length.

In some embodiments, a guide RNA targets a nucleic acid sequence of or of about 20 nucleotides. In some embodiments, a target nucleic acid is less than about 20 nucleotides. In some embodiments, a target nucleic acid is at least or at least about 5, 10, 15, 16, 17, 18, 19, 20, 21, 22, 23, 24, 25, 30, or more nucleotides. In some embodiments, a target nucleic acid is at most or at most about 5, 10, 15, 16, 17, 18, 19, 20, 21, 22, 23, 24, 25, 30 or more nucleotides. In some embodiments, a target nucleic acid sequence is about 20 bases immediately 5′ of the first nucleotide of the PAM. In some embodiments, a guide RNA targets the nucleic acid sequence.

In some embodiments, a guide nucleic acid, for example, a guide RNA, refers to a nucleic acid that can hybridize to another nucleic acid, for example, the target nucleic acid or protospacer in a genome of a cell. In some embodiments, a guide nucleic acid is RNA. In some embodiments, a guide nucleic acid is DNA. In some embodiments, the guide nucleic acid is programmed or designed to bind to a sequence of nucleic acid site-specifically. In some embodiments, a guide nucleic acid comprises a polynucleotide chain and is called a single guide nucleic acid. In some embodiments, a guide nucleic acid comprises two polynucleotide chains and is called a double guide nucleic acid.

In some embodiments, a guide nucleic acid comprises one or more modifications to provide a nucleic acid with a new or enhanced feature. In some embodiments, a guide nucleic acid comprises a nucleic acid affinity tag. In some embodiments, a guide nucleic acid comprises synthetic nucleotide, synthetic nucleotide analog, nucleotide derivatives, and/or modified nucleotides.

In some embodiments, a guide nucleic acid comprises a nucleotide sequence (e.g., a spacer), for example, at or near the 5′ end or 3′ end, that hybridizes to a sequence in a target nucleic acid (e.g., a protospacer). In some embodiments, a spacer of a guide nucleic acid interacts with a target nucleic acid in a sequence-specific manner via hybridization (i.e., base pairing). In some embodiments, a spacer sequence hybridizes to a target nucleic acid that is located 5′ or 3′ of a protospacer adjacent motif (PAM). In some embodiments, the length of a spacer sequence is at least or at least about 5, 10, 15, 16, 17, 18, 19, 20, 21, 22, 23, 24, 25, 30 or more nucleotides. In some embodiments, the length of a spacer sequence is at most or at most about 5, 10, 15, 16, 17, 18, 19, 20, 21, 22, 23, 24, 25, 30 or more nucleotides.

In some embodiments, a guide RNA comprises a dsRNA duplex region that forms a secondary structure. For example, in some embodiments, a secondary structure formed by a guide RNA comprises a stem (or hairpin) and a loop. In some embodiments, a length of a loop and a stem varies. For example, In some embodiments, a loop ranges from about 3 to about 10 nucleotides in length, and a stem ranges from about 6 to about 20 base pairs in length. In some embodiments, a stem comprises one or more bulges of 1 to about 10 nucleotides. In some embodiments, the overall length of a second region ranges from about 16 to about 60 nucleotides in length. For example, in some embodiments, a loop is about 4 nucleotides in length and a stem is about 12 base pairs. In some embodiments, a dsRNA duplex region comprises a protein-binding segment that forms a complex with an RNA-binding protein, such as an RNA-guided endonuclease, e.g. Cas protein.

In some embodiments, a guide RNA comprises a tail region at the 5′ or 3′ end that is essentially single-stranded. For example, In some embodiments, a tail region is not complementarity to any chromosomal sequence in a cell of interest and; in some embodiments, is not complementarity to the rest of a guide RNA. Further, the length of a tail region can vary. In some embodiments, a tail region is more than or more than about 4 nucleotides in length. For example, In some embodiments, the length of a tail region ranges from about 5 to from about 60 nucleotides in length.

In some embodiments, a guide RNA is introduced into a cell as an RNA molecule. For example, in some embodiments, an RNA molecule is transcribed in vitro and/or can be chemically synthesized. In some embodiments, a guide RNA is introduced into a cell as an RNA molecule. In some embodiments, a guide RNA is introduced into a cell in the form of a non-RNA nucleic acid molecule, e.g., DNA molecule. For example, in some embodiments, a DNA encoding a guide RNA is operably linked to promoter control sequence for expression of the guide RNA in a cell or embryo of interest. In some embodiments, an RNA coding sequence is operably linked to a promoter sequence that is recognized by RNA polymerase III (Pol III).

In some embodiments, a DNA molecule encoding a guide RNA is linear. In some embodiments, a DNA molecule encoding a guide RNA is circular.

In some embodiments, a DNA sequence encoding a guide RNA is part of a vector. Some examples of vectors include, but are not limited to, plasmid vectors, phagemids, cosmids, artificial/mini-chromosomes, transposons, and viral vectors. For example, In some embodiments, a DNA encoding an RNA-guided endonuclease is present in a plasmid vector. Other non-limiting examples of suitable plasmid vectors include, but are not limited to, pUC, pBR322, pET, pBluescript, and variants thereof. Further, In some embodiments, a vector comprises additional expression control sequences (e.g., enhancer sequences, Kozak sequences, polyadenylation sequences, transcriptional termination sequences, etc.), selectable marker sequences (e.g., antibiotic resistance genes), origins of replication, and the like.

In some embodiments, when both an RNA-guided endonuclease and a guide RNA are introduced into a cell as DNA molecules, each is part of a separate molecule (e.g., one vector containing fusion protein coding sequence and a second vector containing guide RNA coding sequence). In some embodiments, when both an RNA-guided endonuclease and a guide RNA are introduced into a cell as DNA molecules, both are part of a same molecule (e.g., one vector containing coding (and regulatory) sequence for both a fusion protein and a guide RNA).

In some embodiments, a Cas protein, such as a Cas9 protein or any derivative thereof, is pre-complexed with a guide RNA to form a ribonucleoprotein (RNP) complex. In some embodiments, the RNP complex is introduced into primary immune cells. In some embodiments, introduction of the RNP complex is timed. In some embodiments, the cell can be synchronized with other cells at G1, S, and/or M phases of the cell cycle. In some embodiments, the RNP complex is delivered at a cell phase such that homology directed repair (HDR) is enhanced. The RNP complex can facilitate HDR.

In some embodiments, a guide RNA is modified. In some embodiments, the modifications comprise chemical alterations, synthetic modifications, nucleotide additions, and/or nucleotide subtractions. In some embodiments, the modifications enhance CRISPR genome engineering. In some embodiments, a modification alters chirality of a gRNA. In some embodiments, chirality is uniform or stereopure after a modification. In some embodiments, a guide RNA is synthesized. In some embodiments, the synthesized guide RNA enhances CRISPR genome engineering. In some embodiments, a guide RNA is truncated. In some embodiments, truncation is used to reduce undesired off-target mutagenesis. In some embodiments, the truncation comprises any number of nucleotide deletions. For example, in some embodiments, the truncation comprises 1, 2, 3, 4, 5, 10, 15, 20, 25, 30, 40, 50, or more nucleotides. In some embodiments, a guide RNA comprises a region of target complementarity of any length. For example, in some embodiments, a region of target complementarity is less than 20 nucleotides in length. In some embodiments, a region of target complementarity is more than 20 nucleotides in length.

In some embodiments, a dual nickase approach is used to introduce a double stranded break. In some embodiments, Cas proteins are mutated at known amino acids within either nuclease domains, thereby deleting activity of one nuclease domain and generating a nickase Cas protein capable of generating a single strand break. In some embodiments, a nickase along with two distinct guide RNAs targeting opposite strands are utilized to generate a DSB within a target site (often referred to as a “double nick” or “dual nickase” CRISPR system). This approach may dramatically increase target specificity, since it is unlikely that two off-target nicks will be generated within close enough proximity to cause a DSB.

In some embodiments, a GUIDE-Seq analysis is performed to determine the specificity of engineered guide RNAs. The general mechanism and protocol of GUIDE-Seq profiling of off-target cleavage by CRISPR system nucleases is discussed in Tsai, S. et al., “GUIDE-Seq enables genome-wide profiling of off-target cleavage by CRISPR system nucleases,” Nature, 33: 187-197 (2015).

In some embodiments, a gRNA is introduced at any functional concentration. For example, in some embodiments, a gRNA is introduced to a cell at 10 micrograms. In some embodiments, a gRNA is introduced from 0.5 micrograms to 35 micrograms. In some embodiments, a gRNA is introduced from 0.5, 5, 10, 15, 20, 25, 30, 35, 40, 45, 50, 55, 60, 65, 70, 75, 80, 85, 90, 95, or 35 micrograms.

Delivery of Gene Editing Components

In some embodiments, the nucleases, transcription factors, transgenes, polynucleotides encoding same, and/or any compositions comprising the proteins and/or polynucleotides described herein are delivered to a target cell by any suitable means.

In some embodiments, nucleases of the disclosure are delivered to cells, for example, as mRNA, transcribable DNA, protein, or as part of a ribonucleoprotein complex (RNP). In some embodiments, guide RNAs of the disclosure are delivered as RNA, transcribable DNA, or as part of an RNP. In some embodiments, transcription factors of the disclosure are delivered as proteins, mRNA, or transcribable DNA.

Suitable target cells include, but are not limited, to eukaryotic and prokaryotic cells and/or cell lines. In some embodiments, suitable primary cells include peripheral blood mononuclear cells (PBMC), peripheral blood lymphocytes (PBL), and other blood cell subsets such as, but not limited to, a T cell, a natural killer cell, a natural killer T cell, a monocyte, a monocyte-precursor cell, a hematopoietic stem cell, or a non-pluripotent stem cell.

In some embodiments, the transcription factors, transgenes, and nucleases as described herein are delivered using vectors, for example containing polynucleotide sequences encoding one or more of the proteins disclosed herein. Any vector systems can be used including, but not limited to, plasmid vectors, retroviral vectors, lentiviral vectors, adenovirus vectors, poxvirus vectors; herpesvirus vectors and adeno-associated virus vectors, etc. Furthermore, any of these vectors can comprise one or more transcription factor, nuclease, and/or transgene. Thus, when one or more CRISPR, TALEN, transposon-based, ZEN, meganuclease, or Mega-TAL molecules and/or transgenes are introduced into the cell, CRISPR, TALEN, transposon-based, ZEN, meganuclease, or Mega-TAL molecules and/or transgenes can be carried on the same vector or on different vectors. When multiple vectors are used, each vector can comprise a sequence encoding one or multiple CRISPR, TALEN, transposon-based, ZEN, meganuclease, or Mega-TAL molecules and/or transgenes.

Conventional viral and non-viral based gene transfer methods can be used to introduce nucleic acids encoding engineered CRISPR, TALEN, transposon-based, ZEN, meganuclease, or Mega-TAL molecules and/or transgenes into cells. Such methods can also be used to administer nucleic acids encoding CRISPR, TALEN, transposon-based, ZEN, meganuclease, or Mega-TAL molecules and/or transgenes to cells in vitro. Non-viral vector delivery systems can include DNA plasmids, naked nucleic acid, and nucleic acid complexed with a delivery vehicle such as a liposome or poloxamer. Viral vector delivery systems can include DNA and RNA viruses, which have either episomal or integrated genomes after delivery to the cell.

Methods of non-viral delivery of nucleic acids include electroporation, lipofection, nucleofection, gold nanoparticle delivery, microinjection, biolistics, virosomes, liposomes, immunoliposomes, polycation or lipid: nucleic acid conjugates, naked DNA, mRNA, artificial virions, and agent-enhanced uptake of DNA. Sonoporation using, e.g., the Sonitron 2000 system (Rich-Mar) can also be used for delivery of nucleic acids. Electroporation and/or lipofection can be used to transfect primary cells. Electroporation and/or lipofection can be used to transfect primary immune cells, such as T cells. The skilled worker will appreciate that electroporation and/or lipofection parameters can be optimized to maximize cell viability. This allows the skilled worker to rapidly and efficiently generate the engineered immune cells described herein, despite multiple rounds of electroporation and/or lipofection. For example, engineering a cell with an exogenous TCR and a disruption in a candidate gene may require two separate electroporations (e.g., if the TCR is introduced first and the candidate gene is then disrupted). In some embodiments, electroporation and/or lipofection is performed in a tube or other vessel, and the cells are subsequently transferred to a plate (e.g., a 24-well plate or 96-well plate) for assaying. In this way, cells can also be modified in bulk (e.g., a batch of cells can be modified to express an exogenous receptor such as a TCR) and then divided into smaller samples for further modification (e.g., disruption of candidate genes).

Additional exemplary nucleic acid delivery systems include those provided by AMAXA® Biosystems (Cologne, Germany), Life Technologies (Frederick, Md.), MAXCYTE, Inc. (Rockville, Md.), BTX Molecular Delivery Systems (Holliston, Mass.) and Copernicus Therapeutics Inc. (see for example U.S. Pat. No. 6,008,336). Lipofection reagents are sold commercially (e.g., TRANSFECTAM® and LIPOFECTIN®).

In some cases, a vector encoding for an exogenous TCR can be shuttled to a cellular nuclease. For example, a vector can contain a nuclear localization sequence (NLS). A vector can also be shuttled by a protein or protein complex. In some cases, Cas9 can be used as a means to shuttle a minicircle vector. Cas can comprise an NLS. In some cases, a vector can be pre-complexed with a Cas protein prior to electroporation. A Cas protein that can be used for shuttling can be a nuclease-deficient Cas9 (dCas9) protein. A Cas protein that can be used for shuttling can be a nuclease-competent Cas9. In some cases, Cas protein can be pre-mixed with a guide RNA and a plasmid encoding an exogenous TCR.

In some embodiments, the transfection efficiency of cells with any of the nucleic acid delivery platforms described herein, for example, nucleofection or electroporation, is about 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99%, 99.5%, 99.9%, or more than 99.9%.

Electroporation using, for example, the Neon® Transfection System (ThermoFisher Scientific) or the AMARA® Nucleofector (AMARA® Biosystems) can also be used for delivery of nucleic acids into a cell. Electroporation parameters may be adjusted to optimize transfection efficiency and/or cell viability. Electroporation devices can have multiple electrical wave form pulse settings such as exponential decay, time constant and square wave. Every cell type has a unique optimal Field Strength (E) that is dependent on the pulse parameters applied (e.g., voltage, capacitance and resistance). Application of optimal field strength causes electropermeabilization through induction of transmembrane voltage, which allows nucleic acids to pass through the cell membrane. In some cases, the electroporation pulse voltage, the electroporation pulse width, number of pulses, cell density, and tip type may be adjusted to optimize transfection efficiency and/or cell viability.

In some embodiments, electroporation pulse voltage may be varied to optimize transfection efficiency and/or cell viability. In some embodiments, the electroporation voltage is less than about 500 volts. In some embodiments, the electroporation voltage is least about 500 volts, at least about 600 volts, at least about 700 volts, at least about 800 volts, at least about 900 volts, at least about 350 volts, at least about 135 volts, at least about 1200 volts, at least about 1300 volts, at least about 1400 volts, at least about 1500 volts, at least about 1600 volts, at least about 1700 volts, at least about 1800 volts, at least about 1900 volts, at least about 2000 volts, at least about 235 volts, at least about 2200 volts, at least about 2300 volts, at least about 2400 volts, at least about 2500 volts, at least about 2600 volts, at least about 2700 volts, at least about 2800 volts, at least about 2900 volts, or at least about 3000 volts. In some embodiments, the electroporation pulse voltage required for optimal transfection efficiency and/or cell viability is specific to the cell type. For example, in some embodiments, an electroporation voltage of 1900 volts is optimal (e.g., provide the highest viability and/or transfection efficiency) for macrophage cells. In another example, in some embodiments, an electroporation voltage of about 1350 volts is optimal (e.g., provide the highest viability and/or transfection efficiency) for Jurkat cells or primary human cells such as T cells. In some embodiments, a range of electroporation voltages is optimal for a given cell type. For example, in some embodiments, an electroporation voltage between about 350 volts and about 1300 volts is optimal (e.g., provide the highest viability and/or transfection efficiency) for human 578T cells.

In some embodiments, electroporation pulse width is varied to optimize transfection efficiency and/or cell viability. In some embodiments, the electroporation pulse width is less than about 5 milliseconds. In some embodiments, the electroporation width is at least about 5 milliseconds, at least about 6 milliseconds, at least about 7 milliseconds, at least about 8 milliseconds, at least about 9 milliseconds, at least about 10 milliseconds, at least about 11 milliseconds, at least about 12 milliseconds, at least about 13 milliseconds, at least about 14 milliseconds, at least about 15 milliseconds, at least about 16 milliseconds, at least about 17 milliseconds, at least about 18 milliseconds, at least about 19 milliseconds, at least about 20 milliseconds, at least about 21 milliseconds, at least about 22 milliseconds, at least about 23 milliseconds, at least about 24 milliseconds, at least about 25 milliseconds, at least about 26 milliseconds, at least about 27 milliseconds, at least about 28 milliseconds, at least about 29 milliseconds, at least about 30 milliseconds, at least about 31 milliseconds, at least about 32 milliseconds, at least about 33 milliseconds, at least about 34 milliseconds, at least about 35 milliseconds, at least about 36 milliseconds, at least about 37 milliseconds, at least about 38 milliseconds, at least about 39 milliseconds, at least about 40 milliseconds, at least about 41 milliseconds, at least about 42 milliseconds, at least about 43 milliseconds, at least about 44 milliseconds, at least about 45 milliseconds, at least about 46 milliseconds, at least about 47 milliseconds, at least about 48 milliseconds, at least about 49 milliseconds, or at least about 50 milliseconds. In some embodiments, the electroporation pulse width required for optimal transfection efficiency and/or cell viability is specific to the cell type. For example, in some embodiments, an electroporation pulse width of 30 milliseconds is optimal (e.g., provide the highest viability and/or transfection efficiency) for macrophage cells. In some embodiments, an electroporation width of about 10 milliseconds is optimal (e.g., provide the highest viability and/or transfection efficiency) for Jurkat cells. In some embodiments, a range of electroporation widths is optimal for a given cell type. For example, in some embodiments, an electroporation width between about 20 milliseconds and about 30 milliseconds is optimal (e.g., provide the highest viability and/or transfection efficiency) for human 578T cells.

In some embodiments, the number of electroporation pulses is varied to optimize transfection efficiency and/or cell viability. In some embodiments, electroporation comprises a single pulse. In some embodiments, electroporation comprises more than one pulse. In some embodiments, electroporation comprises 2 pulses, 3 pulses, 4 pulses, 5 pulses 6 pulses, 7 pulses, 8 pulses, 9 pulses, or 10 or more pulses. In some embodiments, the number of electroporation pulses required for optimal transfection efficiency and/or cell viability is specific to the cell type. For example, in some embodiments, electroporation with a single pulse is optimal (e.g., provide the highest viability and/or transfection efficiency) for macrophage cells. In some embodiments, electroporation with a 3 pulses is optimal (e.g., provide the highest viability and/or transfection efficiency) for primary cells. In some embodiments, a range of electroporation widths is optimal for a given cell type. For example, in some embodiments, electroporation with between about 1 to about 3 pulses is optimal (e.g., provide the highest viability and/or transfection efficiency) for human cells.

In some embodiments, a nuclease is added after electroporation. In some embodiments, a nuclease is a DNase or an RNase. In some embodiments, a nuclease reduces cellular clumping and thus increase cellular viability of a sample post genomic modification. In some embodiments, a DNase is added after an electroporation and removed after an incubation period. In some embodiments, an incubation period is from 1 minute up to about 2 weeks. In some embodiments, an incubation is about 5 minutes after an electroporation. In some embodiments, an electroporation is performed with a protein involved in double strand break repair. For example, in some embodiments, introducing a protein involved in double strand break repair improves an efficiency of integration of an exogenous polynucleic acid into a cellular genome.

In some embodiments, electroporating cells comprises administering to a cell a first electroporation step to introduce a nuclease; and a second electroporation step comprising a guide polynucleic acid comprising a region complementary to at least a portion of a gene and an exogenous polynucleic acid comprising a cellular receptor sequence or portion thereof. In some embodiments, a stepwise electroporation of a cell has increased integration of an exogenous polynucleic acid comprising a cellular receptor sequence or portion thereof compared to a comparable cell comprising a single electroporation. In some embodiments, electroporation steps have a period of incubation between each electroporation. For example, in some embodiments, a first electroporation step can have an incubation from about 5 minutes to about 1 week until a second electroporation step is administered to a cell or population of cells. In some embodiments, an incubation time comprises the addition of a nuclease, such as DNase, or a protein involved in double strand break repair. In some embodiments, a protein involved in DNA double strand break repair is added before, during, or after a polynucleic acid that can encode for an exogenous receptor sequence. In some embodiments, a protein or portion thereof involved in DNA double strand break repair is introduced to a population of cells from about 12 hours prior to deliver of a polynucleic acid that encodes a gene or portion thereof. In some embodiments, a protein or portion thereof involved in DNA double strand break repair is introduced to a population of cells from about 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 25 hours, 30 hours, 40 hours, 50 hours, 60 hours, or up to about 80 hours prior to introduction of a polynucleic acid, such as an exogenous TCR, to a population of cells.

In some embodiments, the starting cell density for electroporation is varied to optimize transfection efficiency and/or cell viability. In some embodiments, the starting cell density for electroporation is less than about 1×10⁵cells. In some embodiments, the starting cell density for electroporation is at least about 1×10⁵ cells, at least about 2×10⁵ cells, at least about 3×10⁵ cells, at least about 4×10⁵ cells, at least about 5×10⁵ cells, at least about 6×10⁵ cells, at least about 7×10⁵ cells, at least about 8×10⁵ cells, at least about 9×10⁵ cells, at least about 1×10⁶ cells, at least about 1.5×10⁶ cells, at least about 2×10⁶ cells, at least about 2.5×10⁶ cells, at least about 3×10⁶ cells, at least about 3.5×10⁶ cells, at least about 4×10⁶ cells, at least about 4.5×10⁶ cells, at least about 5×10⁶ cells, at least about 5.5×10⁶ cells, at least about 6×10⁶ cells, at least about 6.5×10⁶ cells, at least about 7×10⁶ cells, at least about 7.5×10⁶ cells, at least about 8×10⁶ cells, at least about 8.5×10⁶ cells, at least about 9×10⁶ cells, at least about 9.5×10⁶ cells, at least about 1×10⁷ cells, at least about 1.2×10⁷ cells, at least about 1.4×10⁷ cells, at least about 1.6×10⁷ cells, at least about 1.8×10⁷ cells, at least about 2×10⁷ cells, at least about 2.2×10⁷ cells, at least about 2.4×10⁷ cells, at least about 2.6×10⁷ cells, at least about 2.8×10⁷ cells, at least about 3×10⁷ cells, at least about 3.2×10⁷ cells, at least about 3.4×10⁷ cells, at least about 3.6×10⁷ cells, at least about 3.8×10⁷ cells, at least about 4×10⁷ cells, at least about 4.2×10⁷ cells, at least about 4.4×10⁷ cells, at least about 4.6×10⁷ cells, at least about 4.8×10⁷ cells, or at least about 5×10⁷ cells. In some embodiments, the starting cell density for electroporation required for optimal transfection efficiency and/or cell viability is specific to the cell type. For example, in some embodiments, a starting cell density for electroporation of 1.5×10⁶ cells is optimal (e.g., provide the highest viability and/or transfection efficiency) for macrophage cells. In another example, in some embodiments, a starting cell density for electroporation of 5×10⁶ cells is optimal (e.g., provide the highest viability and/or transfection efficiency) for human cells. In some embodiments, a range of starting cell densities for electroporation is optimal for a given cell type. For example, in some embodiments, a starting cell density for electroporation between of 5.6×10⁶ and 5×10⁷ cells is optimal (e.g., provide the highest viability and/or transfection efficiency) for human cells such as T cells.

In some embodiments, an electroporation is sequential. For example, in some embodiments, at least one electroporation is performed. In some embodiments, a secondary electroporation is performed from about 30 minutes to about 72 hours after an initial electroporation. In some embodiments, a secondary electroporation is performed from about 30 minutes, 45 minutes, 60 minutes, 1.5 hours, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 24 hours, 25 hours, 26 hours, 27 hours, 28 hours, 29 hours, 30 hours, 31 hours, 32 hours, 33 hours, 34 hours, 35 hours, 36 hours, 37 hours, 38 hours, 39 hours, 40 hours, 45 hours, 50 hours, 55 hours, 60 hours, 65 hours, 70 hours, or up to about 72 hours after an initial electroporation.

In some embodiments, the efficiency of integration of a nucleic acid sequence encoding an exogenous TCR into a genome of a cell with, for example, a CRISPR system, is about 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99%, 99.5%, 99.9%, or more than 99.9%.

In some embodiments, integration of an exogenous polynucleic acid, such as a TCR, is measured using any technique. For example, in some embodiments, integration is measured by flow cytometry, surveyor nuclease assay, tracking of indels by decomposition (TIDE), junction PCR, or any combination thereof. In other cases, transgene integration can be measured by PCR.

### Kits

In one aspect, provided herein are kits that comprise one or more composition or agent described herein. For example, in on aspect a kit described herein a plurality of guide nucleic acid (e.g., a gRNAs) in separate containers, wherein each guide nucleic acid of said plurality binds a different target candidate gene. In some embodiments, said kit contains at least a nuclease (e.g., a nuclease described herein, e.g., an endonuclease). In some embodiments, said kit contains reagents for delivery of said guide nucleic acid and/or said nuclease. In some embodiments, those reagents include viral vectors (e.g., a viral vector described herein) and/or electroporation reagents (e.g., reagents described herein). In some embodiments, said kit comprises cells for use in assays described herein, including, but not limited to, immune cells (e.g., T cells), and/or cancer cells. In some embodiments, said kit comprises reagents to determine a read out from an in vitro assay described herein (e.g., in vitro cytolytic activity of a plurality of T cells). In some embodiments, said kit comprises instructions for conducting an assay described herein.

### EXAMPLES

### Example 1: Isolation and Expansion of T Cells from PBMCs

Isolation of Peripheral Blood Mononuclear Cells (PBMCs) from a LeukoPak

Leukopaks collected from normal peripheral blood were used herein. Blood cells were diluted 3 to 1 with chilled 1×PBS. The diluted blood was added dropwise (e.g., very slowly) over 15 mLs of LYMPHOPREP (Stem Cell Technologies) in a 50 mL conical. Cells were spun at 400×G for 25 minutes with no brake. The buffy coat was slowly removed and placed into a sterile conical tube. The cells were washed with chilled 1×PBS and spun at 400×G for 10 minutes. The supernatant was removed, cells resuspended in media, counted, and viably frozen in freezing media (45 mLs heat inactivated FBS and 5 mLs DMSO).

Isolation of CD3+ T cells

PBMCs were thawed and plated for 1-2 hours in culturing media (RPMI-1640 (with no Phenol red), 20% FBS (heat inactivated), and 1× Gluta-MAX). Cells were collected and counted; the cell density was adjusted to 5×10\hat{ }7 cells/mL and transferred to sterile 14 mL polystyrene round-bottom tubes. Using the EasySep Human CD3 cell Isolation Kit (Stem Cell Technologies), 50 uL/mL of the Isolation Cocktail was added to the cells. The suspension was mixed by pipetting and incubated for 5 minutes at room temperature. After incubation, RapidSpheres were vortexed for 30 seconds, added to the sample at 50 uL/mL, and the sample was mixed by pipetting. The was topped up to 5 mLs for samples less than 4 mLs, or topped up to 10 mLs for samples more than 4 mLs. The sterile polystyrene tube was added to the “Big Easy” magnet, and incubated at room temperature for 3 minutes. The magnet and tube, in one continuous motion, were inverted, pouring off the enriched cell suspension into a new sterile tube.

### Activation and Expansion of CD3+ T Cells

Isolated CD3+ T cells were counted and plated out at a density of 2×10\hat{ }6 cells/mL in a 24 well plate. Dynabeads Human T-Activator CD3/CD28 beads (Gibco, Life Technologies) were added 3:1 (beads:cells) to the cells after being washed with 1×PBS with 0.2% BSA using a dynamagnet. IL-2 (Peprotech) was added at a concentration of 300 IU/mL. Cells were incubated for 48 hours and then the beads were removed using a dynamagnet. Cells were cultured for an additional 6-12 hours before electroporation.

### Example 2: Generation of a Target Vector Carrying a TCR Transgene

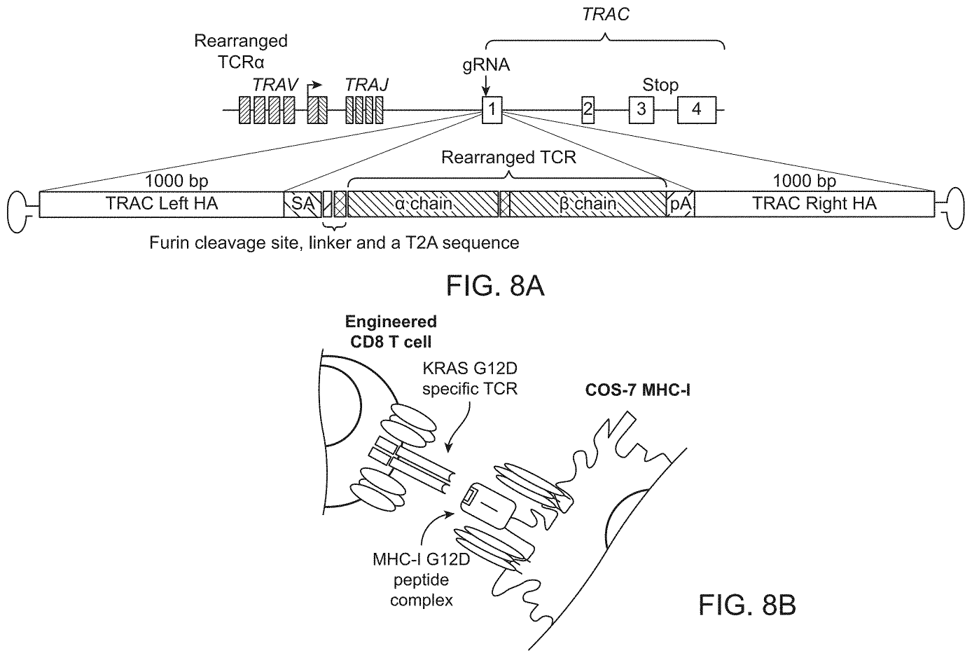

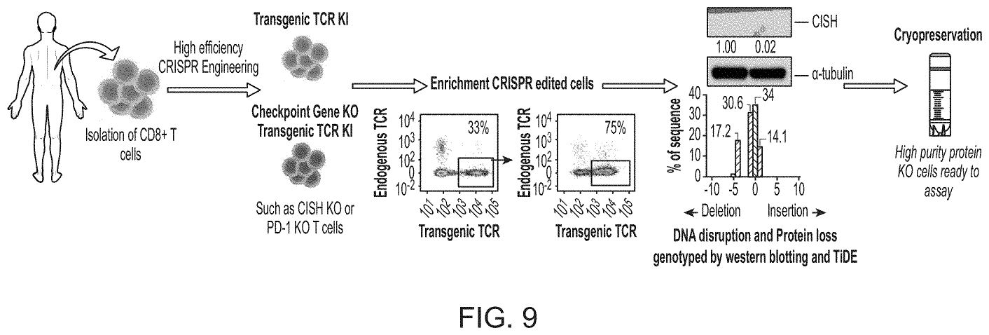

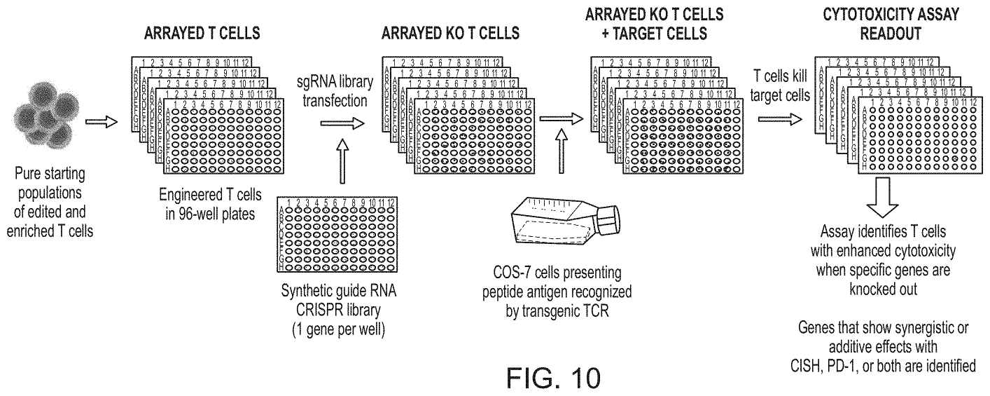

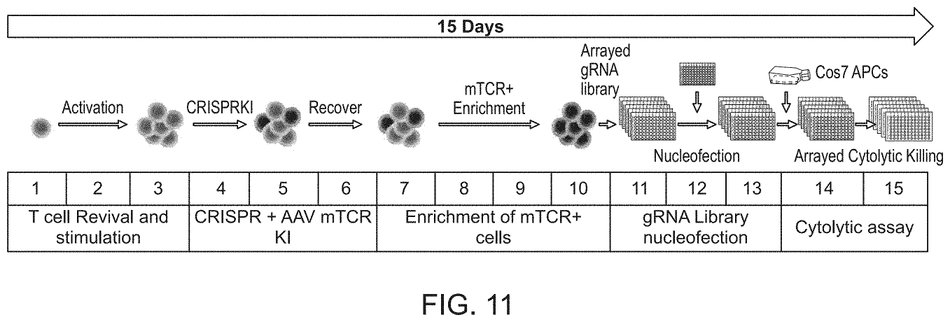

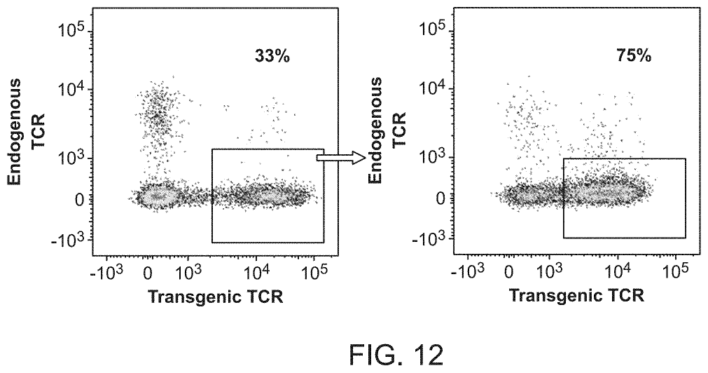

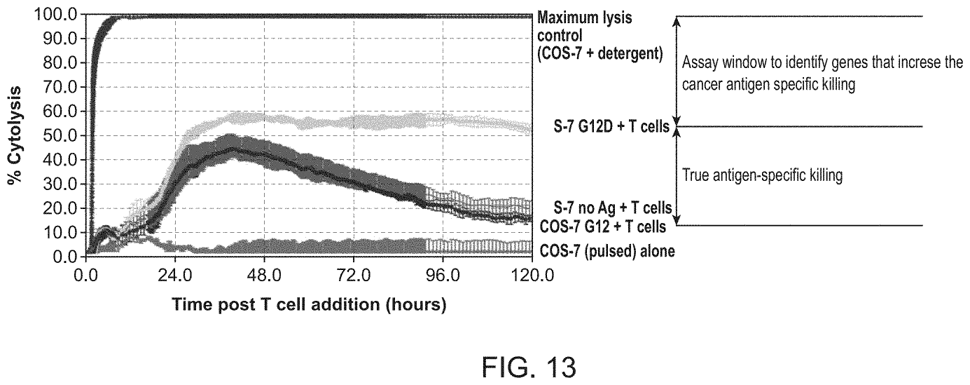

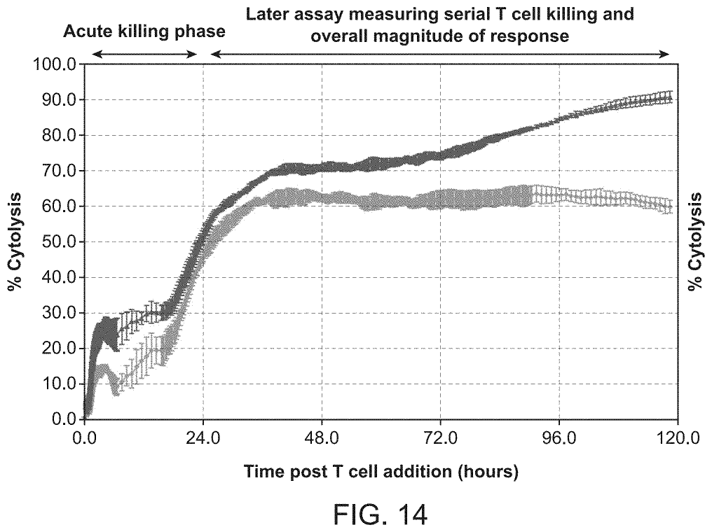

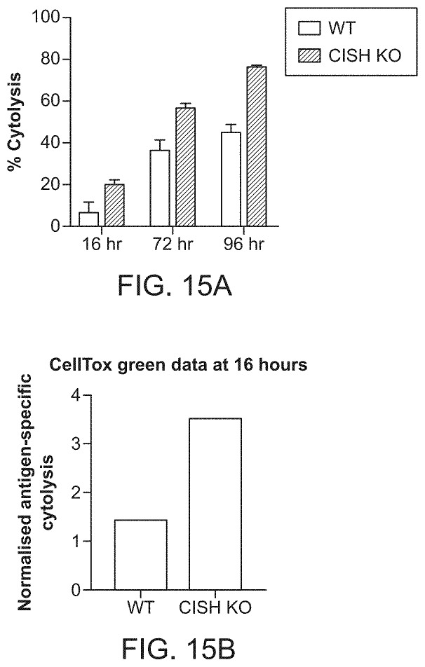

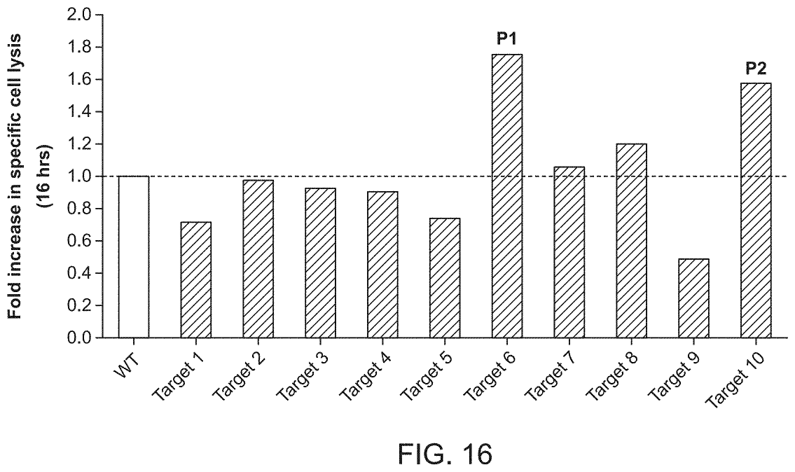

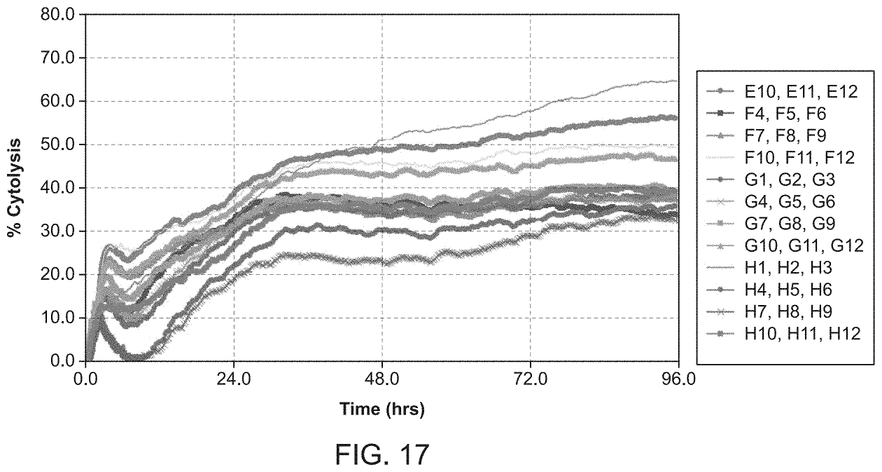

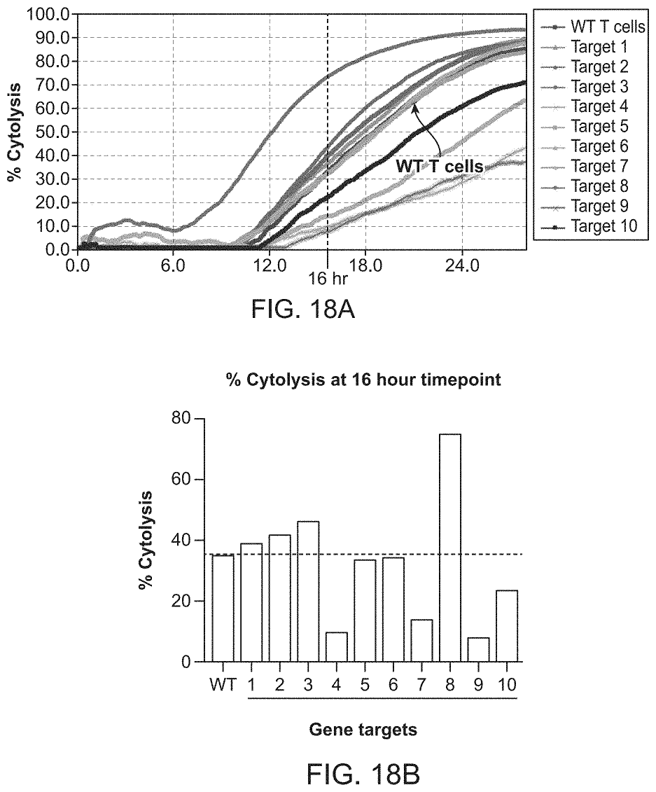

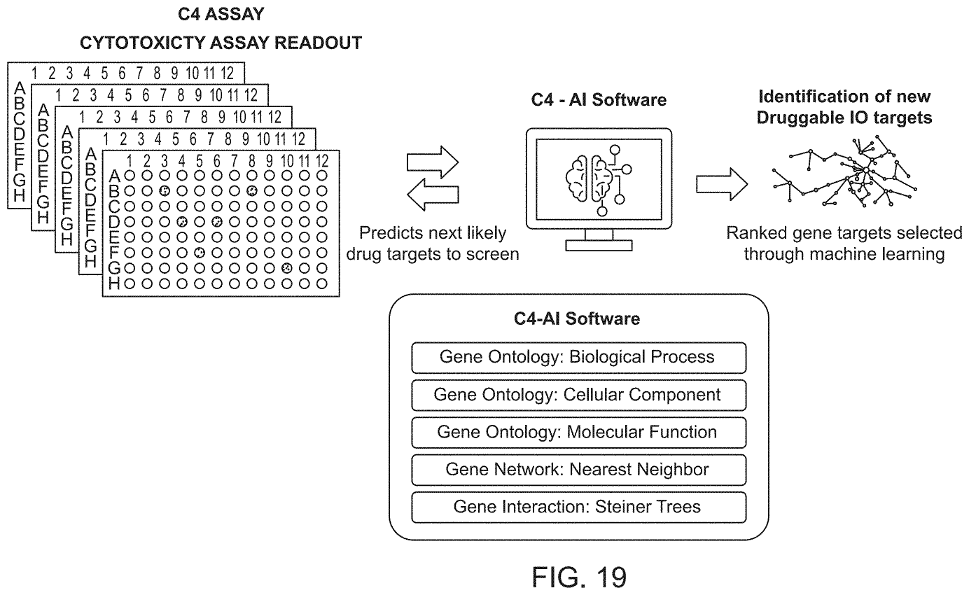

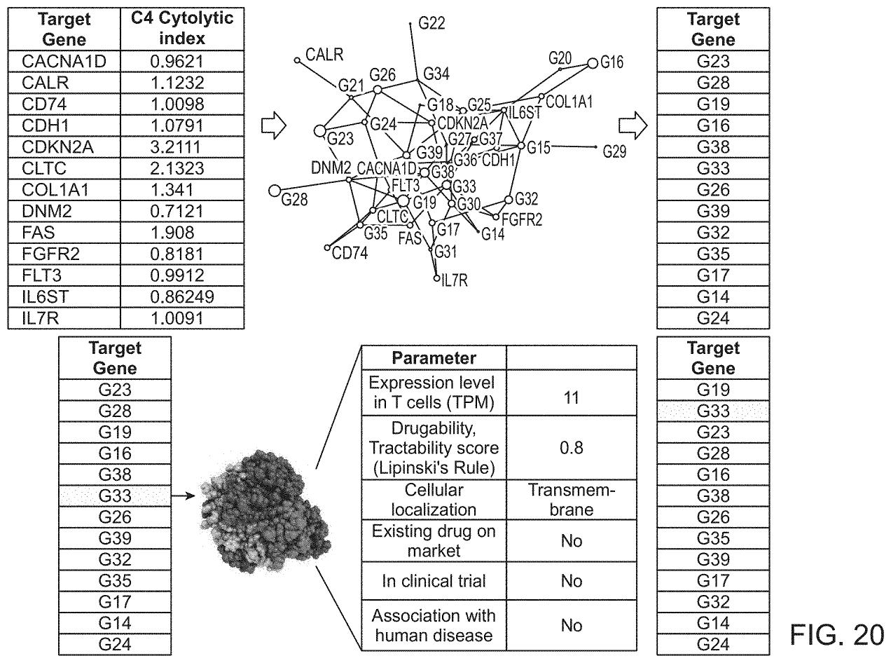

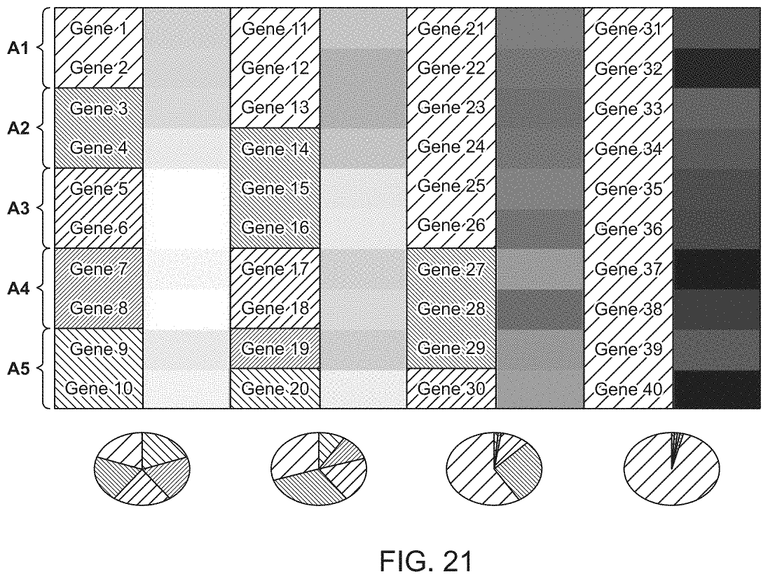

A TCR transgene sequence was acquired and synthesized by IDT as a gBlock. The gBlock was designed with flanking sequences for recombination into a target locus, cloned into pENTR1 via the LR Clonase reaction (Invitrogen) following manufacturer's instructions, and sequence verified. For example, a TCR sequence with a specificity for G12D K-RAS with flanking sequences of 0.5 kb, 1 kb, 2 kb, or 4 kb designed to target integration into the TRAC locus is acquired as a gBlock, cloned into pENTR1 via the LR Clonase reaction (Invitrogen) following manufacturer's instructions, and sequence verified. Intact plasmid or linear DNA (e.g. a PCR product) is used for TCR knockin. An exemplary TCR transgene construct is shown in FIG. 8A, in which the left and right homology arms that flank the alpha and beta chains of the transgenic TCR target the transgene to the TRAC locus.

### Example 3: Transfection of T Cells for TCR Knockin

T cells were electroporated using the Neon Transfection System (10 uL Kit, Invitrogen, Life Technologies). Cells were counted and resuspended at a density of 2×10⁵ cells in 10 uL of T buffer. 1 ug, 0.5 ug, 0.3 ug, 0.2 ug, 0.1 ug, or 0.05 ug of plasmid or short linear DNA encoding the knockin TCR was added. 1 ug Cas9 mRNA and 1 ug of gRNA for the endogenous TCR were also added to the cell mixture. Cells were electroporated at 1400 V, 10 ms, 3 pulses. After transfection, cells were plated in 200 uL culturing media in a 48 well plate, and incubated at 30° C. in 5% CO2 for 24 hrs. After 24 hr recovery, T cells were transferred to antibiotic containing media, cultured at 37° C. in 5% CO2, and subjected to a rapid expansion protocol (REP) over two weeks by stimulating using anti-CD3 in the presence of PBMC feeder cells and IL-2.

### Example 4: Transfection of T Cells for TCR Knockin and Immunomodulatory Gene Knockout

T cells were electroporated using the Neon Transfection System (10 uL Kit, Invitrogen, Life Technologies). Cells were counted and resuspended at a density of 2×10⁵ cells in 10 uL of T buffer. 1 ug, 0.5 ug, 0.3 ug, 0.2 ug, 0.1 ug, or 0.05 ug of plasmid or short linear DNA encoding the knockin TCR were added. 1 ug Cas9 mRNA, 1 ug of gRNA for endogenous TCR, and 1 ug of gRNA for PD-1, CTLA-4, or CISH were also added to the cell mixture. Cells were electroporated at 1400 V, 10 ms, 3 pulses. After transfection, cells were plated in 200 uL culturing media in a 48 well plate, and incubated at 30° C. in 5% CO2 for 24 hrs. After 24 hr recovery, T cells were transferred to antibiotic containing media, cultured at 37° C. in 5% CO2, and subjected to a rapid expansion protocol (REP) over two weeks by stimulating using anti-CD3 in the presence of PBMC feeder cells and IL-2.

### Example 5: Transfection of T Cells for TRAC and Immunomodulatory Gene Knockout and AAV Transduction of T Cells for TCR Knockin

### Day-3: Revival and Stimulation

10% human serum was added to X-VIVO15 media and pre-warmed at 37° C. Human PBMCs were thawed in a water bath. Immediately after thawing cells were resuspended and spun at 300 g for 5-10 minutes. Cells were washed with PBS and counted via a hemocytometer. Cells were then resuspended at one million cells per mL in X-VIVO15+10% Human Serum+300 U/ml IL-2+5 ng/ml IL-7 and IL-15. Anti-CD3 and anti-CD28 Dynabeads were added for stimulation at a ratio of cells:beads of about 1 to 2. Cells were cultured for 3 days.

### Day 0: Removal of Beads, Transfection, and Transduction

Cells were washed with PBS and placed into a DynaMag15 for about 1 minute. Beads were washed off 2 times and then cells pelleted and resuspended in X-VIVO15+Serum+IL-2+IL-7+IL-15 at one million cells per mL. Cells were then cultured at 37° C. for 2 hours before transfection.

For transfections, cells were washed with PBS and pelleted. The cellular pellet was resuspended in T buffer. The required volume of cells (see Table 3) was added into sterile microcentrifuge tubes with Cas9 mRNA and gRNA. For some samples, gRNAs were designed to target the TRAC locus, PD-1, CTLA-4, or CISH. Cells, cas9 mRNA and gRNA were mixed by gentle pipetting. The cell solution was taken up into a Neon tip carefully, ensuring no bubbles were present. Cells were zapped according to programmed conditions. After transfection, cells were cultured at 30° C. for 2 hours before addition of AAV virus comprising DNA encoding the knockin TCR. TCR transgene construct was shown in FIG. 8A, in which the left and right homology arms that flank the alpha and beta chains of the transgenic TCR target the transgene to the TRAC locus.

TABLE 3
Conditions for electroporation with Neon System

| | 10 ul tip | | 35 ul tip | |
|---|---|---|---|---|
| Electrolytic buffer | E | | E2 | |
| Cell number | 2 × 10\hat{ }5 | | 3 × 10\hat{ }6 | |
| Volume for resuspension | ~10 ul per sample | | ~35 ul per sample | |
| Optimal volume in | 12 | ul | 115 | ul |
| microcentrifuge tube | | | | |
| Mass of Cas9 mRNA (L-7206) | 1.5 | ug | 15 | ug |
| Mass of gRNA | 1 | ug | 10 | ug |
| Volume of media | 200 | ul | 3 | ml |

| | | |
|---|---|---|
| Plate size | flat bottom | 6wp |
| | 96wp (or 48wp) | |

TABLE 4
Pulsing conditions

| Pulse voltage | Pulse width | Pulse number |
|---|---|---|
| 1400 | 10 ms | 3 |

Cells were transduced with a MOT of 1e6 of AAV virus particles per cell and cultured at 30° C. overnight.

### Day 1: Media Change

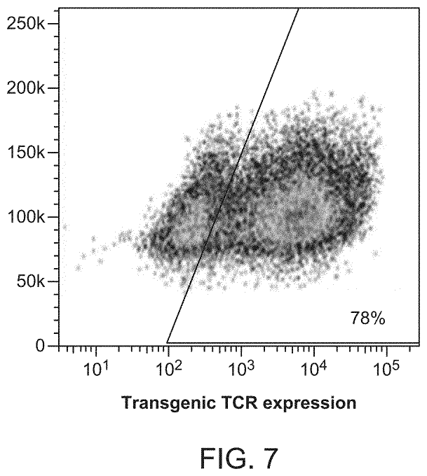

24 hrs post-transduction, cells were removed from the transducing media and transferred into media with phenol red and gentamicin (red media)—with serum and IL-2+IL-7+IL-15. Cells were cultured at 37° C. The efficiency of TCR transgene insertion is shown in FIG. 7, with efficiency reaching 78%.

### Example 6: FACS Enrichment of TCR Knockin T Cells

CRISPR-edited T cells were enriched for TCR knockin, immunomodulatory gene knockout, viable cells, or any combination thereof via fluorescent activated cell sorting (FACS). For example, cells were prepared by washing with chilled 1×PBS with 0.5% FBS and stained with a fluorescently-conjugated antibody specific for the knockin TCR, a fluorescently-conjugated antibody specific for PD-1, and propidium iodide. Cells were then washed and sorted, for example, with a FACSAria™ Fusion sorter (BD Biosciences), to enrich for TCR-knockin positive cells, PD-1 negative cells, viable cells, or any combination thereof. FACS-enriched populations of CRISPR-edited T cells were optionally cryopreserved by freezing in freezing media (90% heat-inactivated FBS, 10% DMSO).

### Example 7: Selective Expansion of TCR Knockin T Cells

CRISPR-edited T cells were enriched for TCR knockin via selective expansion in culture. For example, cells were added to culture plates coated with an anti-TCRβ antibody that binds and activates only T cells comprising the knockin TCR, and additionally coated with anti-CD28 antibody. Cells are incubated for 3-7 days. Enrichment was assessed via flow cytometry. After incubation, the absolute number and relative proportion of T cells expressing the TCR of known antigen specificity was increased.

### Example 8: Confirmation of Loss of Immunomodulatory Gene Protein Expression

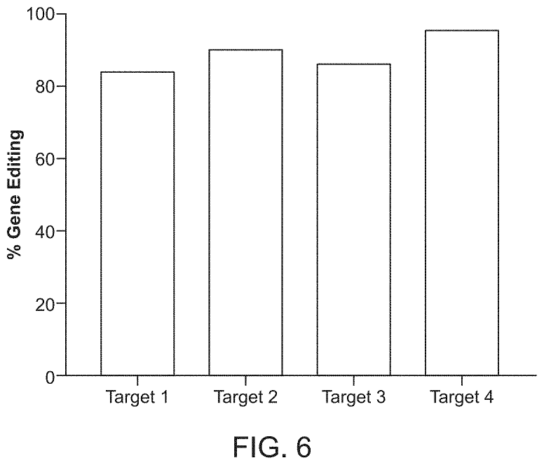

To determine whether nuclease-editing results in the loss of expression of immunomodulatory proteins, flow cytometry or western blot was performed after re-stimulating cells with anti-CD3/CD28 antibodies or dynabeads. T cells were prepared by washing with chilled 1×PBS with 0.5% FBS and stained with a fluorescently-conjugated antibody specific for PD-1 or CTLA-4. Cells were then washed and analyzed by flow cytometry using an LSR II Fortessa (BD Biosciences) and FlowJo software (FlowJo LLC). For assessing the loss of intracellular proteins (e.g. CISH), flow cytometry was performed with permeabilization or western blot. For flow cytometry, loss of protein expression was confirmed by a reduced percentage of positively staining cells or a reduction in mean fluorescence intensity. For Western blot, loss of protein expression was confirmed by a reduction in immunomodulatory gene-specific protein bands assessed via densitometry. The knockout efficiency of four different target genes is shown in FIG. 6, with at least 80% editing efficiency.

### Example 9: Knockout of Candidate Immunomodulatory Genes Via Lentiviral Transduction

FACS-enriched populations of CRISPR-edited T cells generated as described above are thawed in a water bath, resuspended in X-VIVO15 media with 10% FBS at 37° C., pelleted at 300 g for 5-10 minutes, washed with PBS, and resuspended at one million cells per mL in X-VIVO15+10% Human Serum+300 U/ml IL-2+5 ng/ml IL-7 and IL-15. The cells are expanded by stimulation with anti-CD3/CD28 dynabeads at a ratio of cells:beads of about 1:2 for 3 days. Cells are washed with PBS and placed into a DynaMag15 for about 1 minute. Beads are washed off two times, and cells are resuspended in X-VIVO15+Serum+IL-2+IL-7+IL-15 at one million cells per mL, and cultured at 37° C. for 2 hours before transfection.

For transfection of Cas9 mRNA, cells are washed with PBS and pelleted. The cellular pellet is resuspended in T buffer, and the required volume of cells (Table 3) added into sterile microcentrifuge tubes with Cas9 mRNA. Cells and Cas9 mRNA are mixed by gentle pipetting. The cell solution is taken up into a Neon tip carefully, ensuring no bubbles are present. Cells are zapped according to programmed conditions. After transfection, cells are cultured at 30° C. for 2 hours, then transferred to 96-well plates at 35 μL/well.

A LentiArray™ CRISPR gRNA 96 well plate (ThermoFisher) is used, containing lentiviruses encoding gRNAs for candidate immunomodulatory genes. Each well of the 96 well plate can contain gRNAs targeting one candidate immunomodulatory gene, such that each 96 well plate can contain gRNAs for up to 96 different candidate immunomodulatory genes. The LentiArray™ CRISPR gRNA 96 well plate is thawed in a 37° C. water bath, centrifuged at 300×g to collect contents to the bottom of cell wells, and placed on ice. The previously CRISPR-edited T cells are transduced with LentiArray lentiviruses at a multiplicity of infection of 1-10, and optionally centrifuged at 800×g for 30-120 minutes. Transduced T cells are incubated at 37° C., 5% CO2 for three days. The resulting plates contain arrayed knockout T cells, wherein each well contains T cells with a different candidate immunomodulatory gene knocked out. The arrayed T cells can all contain the mutant G12D KRAS-specific TCR that was previously knocked in, with or without additional knockout of a known immunomodulatory gene such as PD-1, CTLA-4, or CISH.

### Example 10: Knockout of Candidate Immunomodulatory Genes Via Nucleofection

Edited T cells expressing TCR of known specificity were washed and distributed across 96 well plates. Cas9 mRNA and synthetic gRNA or RNP were added to the cell mixture, with different gRNAs used to individual genes in individual wells. Nucleofection was performed with pulse code EO-115, in 16 well cuvette strips, with 3×10⁵ cells in each cuvette in a volume of 20 uL of P3 buffer, or using the Neon transfection system according the protocol in Tables 3 and 4.

### Example 11: Co-Culture of Arrayed Knockout T Cells with a Target Cell Line

A target cell line expressing mutant G12D mutant KRAS is seeded in 96 well plates. For example, LS-180 cells (ATCC) are seeded in 96 well plates at 1×10⁴ cells per well. Arrayed T cells with candidate immunomodulatory genes knocked and G12D KRAS-specific TCR knocked in out are added to the LS-180 cells at a ratio of 1 T cell to 1 target cells, 1 T cell to 2 target cells, or 1 T cell to 5 target cells, or 1 T cell to 10 target cells and incubated at 37° C., 5% CO2 for 24-72 hours.

### Example 12: Co-Culture of Arrayed Knockout T Cells with a Target Cell Line Expressing Luciferase and Presenting G12D KRAS Via Human MHC-I

COS-7 cells engineered to express luciferase and human MHC-I are seeded in 96 well plates and pulsed with KRAS G12D. Arrayed T cells with candidate immunomodulatory genes knocked out and G12D KRAS-specific TCR knocked in are added to the COS-7 cells at a ratio 10:1, 5:1, 2:1, 1:1, 1:2, 1:5, or 1:10, and incubated at 37° C., 5% CO2 for 16-48 hours.

### Example 13: Generation of Antigen Presenting Cells for MHC Class II Expression of Target Antigen

Monocyte-derived immature APCs are generated using the plastic adherence method. Briefly, apheresis samples are thawed, washed, adjusted to 5-10×10⁶ cells/mL with neat AIM-V media (Life Technologies) and then incubated at 37° C., 5% CO2. After 90 minutes (min), non-adherent cells are collected, and the flasks are vigorously washed with AIM-V media, and then incubated with AIM-V media for another 60 min. The flasks are then vigorously washed again with AIM-V media and then the adherent cells are incubated with APC media. APC media comprises RPMI containing 5% human serum (collected and processed in-house), 35 U/ml penicillin and 35 μg/ml streptomycin, 2 mM L-glutamine, 800 IU/ml GM-CSF and 800 U/ml IL-4 (media supplements are from LifeTechnologies and cytokines are from Peprotech). On day 3, fresh APC media is added to the cultures.

On days 5-6, immature APCs are matured using a cytokine cocktail comprising LPS, GM-CSF, IL-4, IL-6, IL-1b, C3, C5, and prostaglandin E2 (Sigma) (“maturation cocktail”) for 1-2 days.

### Example 14: Co-Culture of Arrayed Knockout T Cells with Monocyte-Derived Antigen Presenting Cells

Matured APCs are harvested, washed, resuspended at 1×10⁶ cells/mL in cell media supplemented with C3 and C5, then incubated with 1 g/mL of a 25-mer peptide overnight (12-14 h) at 37° C., 5% CO2. After overnight pulsing, APCs are washed 2× with PBS, T-cell media is added and the cells immediately used in co-culture assays. The peptides used are: mutated G12D KRAS peptide, wild-type KRAS peptide, and, as a negative control, mutated ALK. Arrayed T cells with candidate immunomodulatory genes knocked out are added to the antigen-presenting cells at a ratio of 1 T cell to 1 APCs, 1 T cell to 2 APCs, 1 T cell to 5 APCs, or 1 T cell to 10 APCs and incubated at 37° C., 5% CO2 for 24-72 hours.

### Example 15: CytoTox-Glo Cytotoxicity Assay

A CytoTox-Glo™ cytotoxicity assay (Promega) was used to determine the ability of arrayed knockout T cells to kill target cells in the co-culture assays described above. 50 uL of CytoTox-Glo™ reagent was added to all wells of a 96 well plate containing the samples to be assessed. The plate was mixed via orbital shaking and incubated for 15 minutes at room temperature. Extracellular protease from dead cells cleaves a cell-impermeant peptide substrate (AAF-aminoluciferin), resulting in luminescence. A plate reader was used to measure luminescence (experimental dead cell luminescence). 50 uL of lysis reagent was added to each well, the plate was mixed via orbital shaking and incubated for 15 minutes at room temperature. Luminescence was again measured using a plate reader (total luminescence). Viable cell luminescence was calculated from subtracting the experimental dead cell luminescence from total luminescence. T cells with a disrupted immunomodulatory gene can exhibit enhanced cytotoxicity compared to T cells without a disrupted immunomodulatory gene.

### Example 16: HCS LIVE/DEAD Cytotoxicity Assay

An HCS LIVE/DEAD Green Kit (Thermo Fisher) is used to determine the ability of arrayed knockout T cells to kill target cells in the co-culture assays described above. Staining solution is made up by adding 2.1 uL Image-iT® DEAD Green™ viability stain and 40 uL HCS NuclearMask™ Deep Red stain to 6 mL of complete medium per plate to be analyzed. 50 uL of staining solution is added to each well of a 96 well plate containing the samples to be assessed, and the plate is incubated at 37° C. for 30 minutes. Medium is removed, 35 uL of 16% paraformaldehyde is added to each well, and the plate incubated at room temperature for 15 minutes. Fixation solution is removed, cells are washed with PBS, and samples are analyzed for green and deep red fluorescence (excitation/emission 488/515 nm and 638/686 nm, respectively) using a plate reader or fluorescent microscope. The Image-iT DEAD Green viability stain is cell impermeant, but can enter cells with damaged membranes, and exhibit strong fluorescence upon binding to DNA. The HCS NuclearMask reagent is cell permeant and can stain all cells. T cells with a disrupted immunomodulatory gene can exhibit enhanced cytotoxicity compared to T cells without a disrupted immunomodulatory gene.

### Example 17: Luciferase-Based Cytotoxicity Assay

A luciferase assay kit and plate reader are used to determine the ability of arrayed knockout T cells to kill COS-7 cells engineered to express luciferase, and presenting G12D KRAS on human MHC-I as described above. Following co-culture, luciferase assay reagent is added to culture supernatant and luminescence measured immediately using a plate reading luminometer. T cells with a disrupted immunomodulatory gene can exhibit enhanced cytotoxicity compared to T cells without a disrupted immunomodulatory gene.

### Example 18: Electrical Impedance Assay

An electrical impedance assay, such as the xCelligence platform, can be used to identify genes that when inactivated by CRISPR give a robust increase in T cell killing. An adherent target cell line, MHCI engineered COS-7s, were seeded in the xCELLigence 96-well plate at 5,000 COS-7 cells in 150 ul DMEM per well. Their growth was monitored on the machine by an electrical impedance reading (at 37° C. 5% CO2). After 2-3 hours of growth the WT or mutant peptide (G12D KRAS peptide, for example) was added to the COS-7 cells and they were “pulsed” by incubation at 37° C. 5% CO2 for 1-2 hours. The supernatant was then removed and the cells were washed twice in 200 ul PBS before adding 100 ul DMEM growth media and returning to the incubator and xCELLigence machine. Recovery and growth after the peptide pulse were monitored for an hour before CD8+ T cells were added to the COS-7s. 2,500 CD8 T cells in 100 ul of T cell media (x-vivo media+10% human serum+IL2, IL7 and IL15) were added per well. Different effector to target ratios can be used, e.g., an effector target ratio of 0.5:1.

### Example 19: Cytokine Quantification Via ELISA

Cytokines produced in the co-culture assays described above are quantified via Enzyme-linked immunosorbent assay (ELISA). A Human IFN-gamma Quantikine ELISA kit (R&D Systems) is used. 35 uL of assay diluent is added to each well of the ELISA plate. 35 uL of supernatant from co-culture assays, or standard, is added to wells of the ELISA plate. 200 uL of conjugate is added to each well, the plate sealed, and incubated at room temperature for two hours. The Plate is aspirated and washed four times, 200 uL of substrate solution added to each well, and the plate incubated in the dark for 30 minutes. 50 uL of stop solution is added to each well, and absorbance read at 450 nm. T cells with a disrupted immunomodulatory gene can produce a higher quantity of IFN-gamma after co-culture with cells that express or present a cognate antigen, compared to T cells without a disrupted immunomodulatory gene.

### Example 20: Cytokine Quantification Via Multiplex Immunoassay

A plurality of cytokines produced in the co-culture assays are quantified via multiplex immunoassay. A Bio-Plex Pro™ Human Inflammation Assay (Bio-Rad) is used to quantify 37 cytokines. Cell culture supernatants from the co-culture assays are collected after pelleting cells at 1,000×g for 15 minutes at 4° C., and used immediately or stored at about −70° C. until use. 50 uL of coupled beads in assay buffer are added to each well of the Bio-Plex plate. The plate is washed twice, and 50 uL of samples, standards, blanks and controls are added to wells. The plate is sealed and incubated on a shaker at 850 rpm for 1 hr at room temperature. The plate is washed three times, and 25 uL of detection antibodies added to each well, followed by a 30 minute incubation on a shaker at 850 rpm at room temperature. The plate is washed three times, and 50 uL of Streptavidin-PE added to each well, followed by a 10 minute incubation on a shaker at 850 rpm at room temperature. The plate is washed three times, 125 uL of assay buffer added, and the plate shaken at 850 rpm at room temperature for 30 seconds to resuspend beads. The plate is read using Bio-Plex 200 System and Bio-Plex Manager™ software to determine cytokine concentration. T cells with a disrupted immunomodulatory gene can produce greater or lesser quantities of certain cytokines after co-culture with cells that express or present a cognate antigen, compared to T cells without a disrupted immunomodulatory gene.

### Example 21: BrdU T Cell Proliferation Assay

A BrdU Cell Proliferation ELISA Kit (Abcam) is used to measure proliferation of arrayed T cells co-cultured with cells that express or present a cognate antigen. BrdU is added to the wells of the 96 well plate during the co-culture assay. BrdU is incorporated into the DNA of dividing cells. Following co-culture, T cells are resuspended, transferred to a new 96 well plate, and pelleted by centrifugation at 300×g for 5 minutes. Medium is aspirated and 200 uL of fixing solution added per well, followed by a 1 hour incubation at room temperature. The fixing solution fixes and permeabilizes cells, and denatures DNA. The plate is washed three times, 200 uL of anti-BrdU detection antibody is added to each well, and the plate is incubated for 1 hour at room temperature. The plate is washed three times, and 35 uL of peroxidase anti-mouse IgG conjugate added to each well. The plate is washed three times, and 35 uL of TMB peroxidase substrate added to each well. The plate is incubated for 30 minutes in the dark, 35 uL of stop solution added to each well, and a spectrophotometric microtiter plate reader used to measure absorbance at 450 nm. T cells with a disrupted immunomodulatory gene can exhibit enhanced proliferation after co-culture with cells that express or present cognate antigen, compared to T cells without a disrupted immunomodulatory gene.

### Example 22: CFSE T Cell Proliferation Assay

CFSE staining and flow cytometry are used to measure proliferation of arrayed T cells co-cultured with cells that express or present a cognate antigen. Prior to co-culture with cells that express or present a cognate antigen, arrayed T cells are pelleted at 300×g and resuspended in CellTrace CFSE staining solution with 5 uM CFSE (Thermo Fisher). After a 20 minute incubation at 37° C., OpTimizer T Cell Expansion SFM is added to quench any unbound dye. After a 5 minute incubation, cells are pelleted at 300×g, resuspended in T-cell media, and co-cultured with the target cell line or APCs. Following co-culture, T cells are stained with fluorescently-conjugated monoclonal antibodies specific for CD3, CD4 and CD8, and flow cytometric analysis is performed on an LSR Fortessa (BD Biosciences). Data are analyzed using FlowJo software (FlowJo LLC). T cells with a disrupted immunomodulatory gene can exhibit enhanced proliferation after co-culture with cells that express or present cognate antigen, compared to T cells without a disrupted immunomodulatory gene.

### Example 23: Flow Cytometric Analysis of T Cells

Following co-culture, T cells are stained with fluorescently-conjugated monoclonal antibodies specific for IFNγ, IL2, TNFα, CD3, CD4, CD8, CD45RO, CD45RA, CD62L, and CD69. Flow cytometric analysis is performed on an LSR Fortessa (BD Biosciences). Data are analyzed using FlowJo software (FlowJo LLC). T cells with a disrupted immunomodulatory gene can exhibit an increased propensity for differentiation into T_{EM}, an increased propensity for differentiation into T_{CM}, increased expression of activation markers, or a combination thereof, compared to T cells without a disrupted immunomodulatory gene.

### Example 24: Functional Validation

Genes identified as potential immunomodulatory genes from lentiviral gRNA arrays are validated by further experiments. Guide RNAs (gRNAs) are designed to the desired region of a gene. Multiple primers to generate gRNAs are chosen based on the highest ranked values determined by off-target locations. The gRNA sequences can be modified to contain 2-O-Methyl 3phosphorothioate additions. The gRNAs are ordered in oligonucleotide pairs: 5′-CACCG-gRNA sequence-3′ and 5′-AAAC-reverse complement gRNA sequence-C-3′.

gRNAs are cloned together using a target sequence cloning protocol. Briefly, the oligonucleotide pairs are phosphorylated and annealed together using T4 PNK (NEB) and 10×T4 Ligation Buffer (NEB) in a thermocycler with the following protocol: 37° C. 30 minutes, 95° C. 5 minutes and then ramped down to 25° C. at 5° C./minute. pENTR1-U6-Stuffer-gRNA vector (made in house) is digested with FastDigest BbsI (Fermentas), FastAP (Fermentas) and 10× Fast Digest Buffer are used for the ligation reaction. The digested pENTR1 vector is ligated together with the phosphorylated and annealed oligo duplex (dilution 1:200) from the previous step using T4 DNA Ligase and Buffer (NEB). The ligation is incubated at room temperature for 1 hour and then transformed and subsequently mini-prepped using GeneJET Plasmid Miniprep Kit (Thermo Scientific). The plasmids are sequenced to confirm the proper insertion.

HEK293T cells are plated out at a density of 1×10\hat{ }5 cells per well in a 24 well plate. 150 uL of Opti-MEM medium is combined with 1.5 ug of gRNA plasmid, and 1.5 ug of Cas9 plasmid. Another 150 uL of Opti-MEM medium is combined with 5 ul of Lipofectamine 2000 Transfection reagent (Invitrogen). The solutions are combined together and incubated for 15 minutes at room temperature. The DNA-lipid complex is added dropwise to wells of the 24 well plate. Cells are incubated for 3 days at 37° C. and genomic DNA is collected using the GeneJET Genomic DNA Purification Kit (Thermo Scientific). Activity of the gRNAs is quantified by a Surveyor Digest, gel electrophoresis, and densitometry (Guschin, D. Y., et al., “A Rapid and General Assay for Monitoring Endogenous Gene Modification,” Methods in Molecular Biology, 649: 247-256 (2010)).

gRNAs showing high efficiency in generating double-stranded breaks are used to disrupt candidate immunomodulatory genes as outlined in other examples. The resulting TCR-knockin, candidate immunomodulatory knockout T cells are evaluated in functional assays as outlined in other examples.

### Example 25: Ranking Candidate Immunomodulatory Genes Based on Screening Assays and Other Weighted Parameters

Candidate immunomodulatory genes were disrupted in T cells, T cells were co-cultured with target cells, and a screening assay (e.g., a cytotoxicity assay) was run as outlined in above examples. As an output of the screening assay, numerical data was obtained for each disrupted gene (reflecting, for example, cytotoxicity). For each gene, data was pulled from relevant databases, and an algorithm was used to generate a ranked list of screened genes wherein genes were ranked based on the following logic parameters: (a) numerical data from the screening assay (e.g. cytotoxicity of the knockout T cell); (b) expression of the gene in human T cells, (yes/no, low/medium/high, or numeric value); (c) subcellular localization of the gene's protein product (nuclear/cytoplasmic/cell surface) (d) designation of the gene in the ‘druggable genome’ (yes/no); (e) known association of loss of function of the gene with human disease (yes/no); (f) predicted efficiency of CRISPR gRNA used to disrupt candidate gene (ranked order); (g) existing drugs or drugs in development known to target the gene (yes/no); (h) a known loss of function phenotype for the gene in mice (yes/no). The contribution the logic parameters to the final rankings are weighted as follows: highest weighting: (a); high weighting: (b) and (c); medium weighting: (d) and (e); lower weighting: (f), (g), and (h). An illustrative algorithm workflow is provided in FIG. 5A. The ranked list of screened genes is used to prioritize target genes for validation and further investigation.

### Example 26: Iterative Selection of Candidate Immunomodulatory Genes to Screen

A ranked list of genes was generated as described in example 24. The top ranked genes were queried against databases in order to determine the following characteristics for each gene: (a) membership in a gene family; (b) predicted or known gene function; and (c) participation in a signaling pathway. For each identified family, function, and signaling pathway, a genome-wide search is conducted for other genes of the same family, function, or signaling pathway. A list is generated comprising all identified genes, and an algorithm is used to rank the list based on the number of characteristics (family/function/signaling pathway) shared with the top ranked genes from the screening assay.

Top ranking genes from the list are then disrupted in T cells, T cells are co-cultured with target cells, and a screening assay run as outlined in above examples (e.g., a cytotoxicity assay).

An algorithm was used to correlate the results of the screening assay with the prevalence of hits for each characteristic described above, and weightings are calculated for each characteristic based on the strength of correlation. Characteristic weightings are applied to re-rank the list of identified genes.

The gene disruption, co-culture, screening assay, correlation calculations, weighting calculations, and list re-ranking steps are repeated iteratively to identify and screen new sets of candidate immunomodulatory genes.

An illustrative algorithm workflow is provided in FIG. 5B.

Previously screened genes can be omitted from subsequent rounds to minimize redundancy.

### Example 27: Identification of Druggable Immunomodulatory Genes Related to Candidate Genes that are Poor Drug Targets

Candidate immunomodulatory genes are disrupted in T cells, T cells are co-cultured with target cells, and a screening assay (e.g., a cytotoxicity assay) is run as outlined in above examples. As an output of the screening assay, numerical data is obtained for each disrupted gene (reflecting, for example, cytotoxicity).

The screened genes are queried against databases in order to determine the following characteristics for each gene: (a) subcellular localization of the gene's protein product (nuclear/cytoplasmic/cell surface); and (b) designation of the gene in the ‘druggable genome’ (yes/no). Genes with a nuclear localization, cytoplasmic localization, or ‘no’ designation for the druggable genome are selected for further analysis.

An algorithm is used to generate a ranked list of the selected genes, wherein genes are ranked based on the following logic parameters: (a) numerical data from the screening assay (e.g. cytotoxicity of the knockout T cell); (b) expression of the gene in human T cells, (yes/no, low/medium/high, or numeric value); (c) known association of loss of function of the gene with human disease (yes/no); (d) predicted efficiency of CRISPR gRNA used to disrupt candidate gene (ranked order); (e) existing drugs or drugs in development known to target the gene (yes/no); (f) a known loss of function phenotype for the gene in mice (yes/no). The contribution the logic parameters to the rankings are weighted as follows: highest weighting: (a); high weighting: (b); medium weighting: (c); lower weighting: (d), (e), and (f).

The top ranked genes are queried against databases in order to determine the following characteristics for each gene: (a) membership in a gene family; and (b) participation in a signaling pathway. For each identified family and signaling pathway, a genome-wide search is conducted for other genes that are in members of the same family or upstream within the same signaling pathway. A list is generated comprising all identified genes, and an algorithm is used to rank the genes based on the following logic parameters: (a) expression of the gene in human T cells, (yes/no, low/medium/high, or numeric value); (b) subcellular localization of the gene's protein product (nuclear/cytoplasmic/cell surface) (c) designation of the gene in the ‘druggable genome’ (yes/no); (d) known association of loss of function of the gene with human disease (yes/no); (e) predicted efficiency of CRISPR gRNA used to disrupt candidate gene (ranked order); (f) existing drugs or drugs in development known to target the gene (yes/no); (g) a known loss of function phenotype for the gene in mice (yes/no). The contribution the logic parameters to the final rankings are weighted as follows: high weighting: (a) and (b); medium weighting: (c) and (d); lower weighting: (e), (f), and (g). The ranked list of genes is used to prioritize candidate genes for the next round of screening.

Top ranking genes from the list are then disrupted in T cells, T cells are co-cultured with target cells, and a screening assay run as outlined in above examples (e.g., a cytotoxicity assay). The prior steps in this example can then be repeated iteratively to identify and screen new sets of candidate immunomodulatory genes. An illustrative algorithm workflow is provided in FIG. 5C. Previously screened genes can be omitted from subsequent rounds to minimize redundancy.

1. A method of screening a plurality of single candidate genes, said method comprising:
a. expressing an exogenous cellular receptor, or a functional fragment thereof, in a plurality of separate populations of immune cells, wherein each population comprises a plurality of immune cells;
b. introducing into each of said separate populations of immune cells a CRISPR system that comprises:
i. a guide nucleic acid that binds a portion of a single candidate gene, wherein said single candidate gene is different for each of said separate populations of immune cells; and
ii. an exogenous nuclease, or a nucleic acid encoding said exogenous nuclease;
thereby generating a plurality of separate populations of engineered immune cells that comprise a genomic disruption in said single candidate gene, wherein said genomic disruption that suppresses expression of said single candidate gene;
c. performing an in vitro assay that comprises contacting said plurality of engineered immune cells with a plurality of cells expressing a cognate antigen of said exogenous cellular receptor or said functional fragment thereof in vitro; and
d. obtaining a readout from said in vitro assay, to thereby determine an effect of said genomic disruption that suppresses expression of said single candidate gene on said plurality of separate populations of engineered immune cells.

2. The method of claim 1, wherein said readout comprises determining a level of cytolytic activity of each of said plurality of separate populations of engineered immune cells.

3. (canceled)

4. The method of claim 1, wherein said readout comprises determining a level of proliferation of each of said plurality of separate populations of engineered immune cells.

5. (canceled)

6. The method of claim 1, wherein said readout comprises determining a level of a factor expressed by each of said plurality of separate populations of engineered immune cells.

7. The method of claim 6, wherein said factor is a protein.

8. The method of claim 7, wherein said protein is secreted from said population of engineered immune cells.

9. The method of claim 7, wherein said protein is a cytokine or chemokine.

10. (canceled)

11. (canceled)

12. (canceled)

13. (canceled)

14. (canceled)

15. (canceled)

16. (canceled)

17. (canceled)

18. (canceled)

19. The method of claim 1, wherein said exogenous cellular receptor is integrated into an AAVS site, CCR5, or hROSA26.

20. (canceled)

21. (canceled)

22. (canceled)

23. (canceled)

24. (canceled)

25. (canceled)

26. (canceled)

27. (canceled)

28. The method of claim 1, wherein said nuclease is an endonuclease.

29. The method of claim 28, wherein said endonuclease is selected from the group consisting of Cas1, Cas1B, Cas2, Cas3, Cas4, Cas5, Cash, Cas7, Cas8, Cas9, Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx1S, Csf1, Csf2, CsO, Csf4, Cpf1, c2c1, c2c3, and Cas9HiFi.

30. The method of claim 29, wherein said endonuclease is Cas9.

31. The method of claim 1, wherein said guide nucleic acid is a guide ribonucleic acid (gRNA).

32. The method of claim 1, wherein said guide nucleic acid comprises a phosphorothioate (PS) linkage, a 2′-fluoro (2′-F) modification, a 2′-O-methyl (2′-O-Me) linkage, a 2-O-Methyl 3phosphorothioate linkage, a S-constrained ethyl (cEt) modification, or any combination thereof

33. (canceled)

34. (canceled)

35. The method of claim 1, wherein said exogenous cellular receptor is introduced using a viral vector.

36. (canceled)

37. The method of claim 35, wherein said viral vector comprises an AAV vector selected from the group consisting of a recombinant AAV (rAAV) vector, a hybrid AAV vector, a chimeric AAV vector, a self-complementary AAV (scAAV) vector, a modified AAV vector, and any combination thereof.

38. The method of claim 37, wherein said AAV vector is a chimeric AAV vector.

39. The method of claim 38, wherein said chimeric AAV vector comprises a modification in at least one AAV capsid gene sequence.

40. The method of claim 1, wherein said exogenous cellular receptor is a T-cell receptor (TCR), B cell receptor (BCR), NK cell receptor, dendritic cell receptor, monocyte receptor, macrophage receptor, neutrophil receptor, eosinophil receptor, or a chimeric antigen receptor (CAR).

41. (canceled)

42. (canceled)

43. (canceled)

44. (canceled)

45. (canceled)

46. (canceled)

47. (canceled)

48. (canceled)

49. (canceled)

50. (canceled)

51. (canceled)

52. (canceled)

53. (canceled)

54. (canceled)

55. (canceled)

56. (canceled)

57. The method of claim 1, wherein each of said populations of engineered immune cells comprises a plurality of T cells, tumor infiltrating lymphocytes (TILs), NK cells, B cell, dendritic cells, monocytes, macrophages, neutrophils, or eosinophils.

58. (canceled)

59. (canceled)

60. (canceled)

61. (canceled)

62. (canceled)

63. (canceled)

64. (canceled)

65. (canceled)

66. The method of claim 1, wherein said each of said populations of engineered immune cells comprises a transgene that encodes for a protein that improves immunomodulatory function of said engineered immune cells.

67. (canceled)

68. (canceled)

69. The method of claim 66, wherein said transgene is integrated into a site comprising an AAVS site, CCR5, or hROSA26.

70. (canceled)

71. (canceled)

72. (canceled)

73. (canceled)

74. (canceled)

75. (canceled)

76. (canceled)

77. (canceled)

78. (canceled)

79. (canceled)

80. (canceled)

81. (canceled)

82. (canceled)

83. (canceled)

84. (canceled)

85. (canceled)

86. A composition comprising a plurality of separate populations of immune cells, wherein each separate population of immune cells comprises a plurality of immune cells that i) express an exogenous cellular receptor; and ii) comprise a CRISPR system that comprises a guide nucleic acid that binds a portion of a single candidate gene, wherein said single candidate gene is different for each of said separate populations of immune cells; and an exogenous nuclease, or a nucleic acid encoding said exogenous nuclease.

87. The composition of claim 86, wherein said population of said plurality of immune cells of each separate population comprises a genomic disruption in said single candidate gene.

88. (canceled)

89. (canceled)

90. (canceled)

91. A composition comprising a plurality of separate cell populations that each comprise i) a plurality of immune cells that express an exogenous cellular receptor and ii) cells that express a cognate antigen of said exogenous cellular receptor; wherein each of said plurality of immune cells comprises an altered genome sequence of a single candidate gene, and wherein said single candidate gene is different for each of said separate cell populations.

92.-194. (canceled)
